(19) United States
(12) Patent Application Publication
(10) Pub. No.: US 2011/0263487 A1
(43) Pub. Date: Oct. 27, 2011

(54) Plant Production and Delivery System for Recombinant Proteins as Protein-Flour or Protein-Oil Compositions

(75) Inventor: Richard B. Meagher, Athens, GA (US)

(73) Assignee: University of Georgia Research Foundation, Inc., Athens, GA (US)

(21) Appl. No.: 12/809,470

(22) PCT Filed: Dec. 22, 2008
(86) PCT No.: PCT/US08/13953

§ 371 Date: Jul. 13, 2011

Related U.S. Application Data
(60) Provisional application No. 61/008,340, filed on Dec. 20, 2007.

(51) Int. Cl. A01N 37/18; C12N 9/96; A01P 7/04; C07K 19/00

(52) U.S. Cl. 514/4.5; 530/377; 435/188

(57) ABSTRACT

The present invention relates to a plant flour material comprising a ground, dried plant material, wherein the plant material contains a recombinant protein comprises a bactericidal or an insecticidal protein toxin, or combinations thereof. The present invention further relates to a seed oil body composition comprising a seed oil body fusion, said oil body fusion comprising an oil body protein fused to a recombinant protein comprising a bactericidal or an insecticidal protein toxin. The present invention provides for a method of abating or controlling the population of mosquitoes comprising administering to a water source suspected of containing mosquito larvae an effective amount of seed oil body preparation comprising one or more Bti toxins, optionally in combination with one or more BtBs. Additionally, the present invention provides for a method of controlling or abating a pathogenic microbial population comprising administering to an insect population, which serves as a host for the pathogenic microbial population, a recombinant plant material containing a recombinant protein comprising an bactericidal protein toxin.

### BACKGROUND OF THE INVENTION

1. Field of the Invention

The present invention relates to the production of recombinant proteins and peptides in plants and the use of materials generated from such plants, such as protein flours or protein oil body fusions, to control pest insect populations on a large-scale.

2. Background Art

Insects are problematic in all areas of the world, ranging from agricultural crop destruction to the transmission of diseases. For example, a number of moth and caterpillar species are major crop pests. Similarly, mosquitoes and certain beetle populations are well recognized to transmit and carry pathogenic diseases such as those caused by viral, protozoan, or bacterial pathogens. A number of prior approaches have been employed to help control insects that mainly involve the use of chemical pesticides. Chemical pesticides are problematic and can be toxic to people and cause harm to the environment. Recently, transgenic plants containing biological insecticidal agents have been used to modify the plant crop itself to increase production yield. The incorporation of biological insecticidal agents within the plant itself cuts down on chemical pesticide use but some segments of the population have raised concerns over the safety of genetically modified foods. The present invention provides compositions, methods and the like to effectively and efficiently control both agricultural pests that destroy crops and insects involved in the destruction of crops and in the transmission of infectious diseases using materials derived from genetically modified plants.

Bacillus thuringiensis (B.t.) is a facultative anaerobic, Gram-positive, motile, spore-forming bacterium. The cry gene family encodes for the B.t. toxin. Strain and gene isolation have led to the discovery of over 250 cry genes. B.t. has been used by farmers as an insecticide to control lepidopteran and coleopteran pests for more than 30 years. B.t. species produce a variety of toxic proteins (B.t.-toxins) that are effective insecticidal agents, and are widely commercially used. The insecticidal agent of the B.t. bacterium is a protein, which has such limited animal toxicity that it can be used on human food crops on the day of harvest. To non-targeted organisms, the B.t. toxin is a digestible non-toxic protein. B.t. proteins safely control many insect species when used either as formulations of native and/or engineered B.t. strains, and reduce chemical pesticide usage (Betz et al. (2002) Regulatory Toxilcology and Pharmacology 32: 156-173.)

Since the 1980s, people have attempted to improve B.t. In spite of the high level of insect larval specificity and toxicity of B.t.s, some lepidopteran species are not highly susceptible to B.t. Receptors in the midgut of insects bind to B.t. when they ingest the protein. (Luo et al. (1999) Appl Environ Microbiol 65: 457-64); (McNall and Adang, (2003) Insect Biochem. Mol Biol 33: 999-1010); (Hua et al. (2004) J Biol Chem 279: 28051-28056). Most Cry toxin-binding midgut proteins identified to-date belong to either the cadherin-like protein family or the aminopeptidase family. B.t. toxicity appears to be mediated by the expression of receptor proteins in the insect gut (Luo et al. (1997) Insect Biochem. Mol Biol 27: 735-743) and (Jurat-Fuentes and Adang (2006a) J Invertebr Pathol 92: 166-171.)

Combining B.t. proteins with peptide fragments of the receptors, also known as BtBs (B.t.Boosters), from the gut that bind to B.t. can increase the susceptibility of the insects to the B.t. toxin. One B.t. receptor, B.t.-R1 that mediates the activity of Cry1A toxins was isolated from Manduca sexta (tobacco hornworm) (Hua et al. (2004) J Biol Chem 279: 28051-28056) and (Chen et al. (2007) Proc Natl Acad Sci USA 104: 13901-13906.) Native B.t.-R1 is a large complex 220 kDa protein composed of 12 cadherin repeats, a membrane proximal extracellular domain (MPED), a membrane-spanning domain, and a intracellular domain (Francis and Bulla (1997) Insect Biochem. Mol Biol 27: 541-50) and (Hua et al. (2001) Appl Environ Microbiol 67: 872-879.) Bacterially produced fragments of B.t.-R1 have been reported to be fed along with B.t. toxins like Cry1Ac to enhance toxin insecticidal activities (Hua et al. (2004) J Biol Chem 279: 28052-28056); (Jurat-Fuentes and Adang (2006b) Biochemistry 45: 9688-96895); (Jurat-Fuentes and Adang (2006a) J Invertebr Pathol 92: 166-171.); and (Chen et al. (2007) Proc Natl Acad Sci USA 104: 13901-13906.)

Insects with low receptor levels are naturally less susceptible to the toxicity of B.t.s. When portions of these receptor B.t. peptide fragments proteins are added along with the appropriate B.t. toxin, B.t. toxicity has been reported to be enhanced 10-fold and the target insect range has been reported to be expanded to larvae of less susceptible species (Chen et al. (2007) Proc Natl Acad Sci USA 104: 13901-13906.) B.t.Boosters have no inherent toxicity of their own to insects or other animal species.

### Litter Beetles

Billions of chickens are produced annually in the United States and global chicken production is rapidly increasing. Bacterial, protozoan, and viral pathogens are a constant health hazard during large-scale poultry production. The strong and mounting evidence that bird to human transfer of disease is responsible for several past pandemic diseases heightens concern that poultry diseases threaten public health. Among the bacterial chicken pathogens of greatest concern are Salmonella species, which can contaminate both the meat and eggs to be consumed by humans.

The litter beetle (lesser mealworm, darkling beetle), Alphitobius diaperinus Panzer (Coleoptera: Tenebrionidae), is a serious pest in the poultry industry and are well known for eating feed, disturbing chickens, harboring diseases, and causing damage to housing. Litter beetles and a few other Coleopteran-species act as vectors for protozoan, bacterial, and viral diseases of chickens and turkeys resulting in significant economic loss. These beetles inhabit the litter, wood, Styrofoam, fiberglass, and polystyrene insulation panels of chicken houses. Larvae and adult beetles thrive both on bird droppings and on grains used as chicken feed and can reach incredibly high numbers, exceeding 2×10⁶ per 20,000 sq ft broiler house and it is not unusual to have over 1,000 beetles per square yard.

In addition to eating large amounts of feed, litter beetles have been associated with transmitting many diseases, including infectious bursal disease virus (IBDV), Marek's, infectious laryngotracheitis (LT), E. coli, Salmonella, Dermatitis, Necrotic Enteritis, Aspergillosis, avian influenza, botulism and Coccidiosis. Essentially any disease agent that the beetles come into contact with can be transmitted throughout the poultry house. In addition to disease transmission other associated health problems in humans result because the beetles produce high active benzoquinones as a defense against predation. Quinones can be hazardous to human health and can cause symptoms of asthma, headaches, dermatitis angiodema, rhinitis, and erythema. Exposure to quinone vapors can also result in conjunctivitis and corneal ulceration.

None of the currently available insecticides provide satisfactory control of litter beetles (Miller (1990) Poult Sci 69: 1281-1284); (Salin et al. (2003) Proc Natl Acad Sci USA 93: 3182-3187); and (Calibeo-Hayes et al. (2005) J Econ Entamol 98: 229-235.) Nor do they provide the possibility of insecticidal application in the presence of the chickens. Some resistance to insecticides has also been observed. A more effective control of disease in poultry is therefore needed.

### Aquatic Insects

Mosquitoes are vectors for numerous blood-borne diseases, including malaria, yellow fever, West Nile virus, filariasis, Japanese encephalitis, and dengue fever and cause millions of deaths worldwide annually. West Nile virus has become a particular concern in the USA in the last decade. Preventative measures have focused on controlling both the adult and larval stages of mosquitoes, but these have met with mixed success in many countries such as Brazil (Killeen et al. (2002) Lancet Infect Dis 2: 618-627) and (Killeen (2003) Lancet Infect Dis 3: 663-666.) Bacillus thuringiensis israelensis (B.t.i) is an insecticide that can control mosquitoes and other dipteran species.

Mosquito larvae are filter-feeders that inhabit open-water environments, including: marshes, ponds, and eddies along stream and river-banks. Depending upon species, larvae live at various depths in the water column. Of all vectors for zoonotic transmission of infectious agents, mosquitoes are one of the more difficult to control. Mosquito larvae are known to be susceptible to the B.t.i toxin. Other dipteran species like the black fly (Simulium damnosum), which is a prominent vector for African river blindness (onchocerciasis), can also be controlled by B.t.i-related toxins (Dadzie et al. (2003) Filaria J 2: 2.)

There have been a number of commercial formulations that attempt to disperse the B.t.i-producing bacteria or B.t.i toxins in micro-pellets or micro-droplets to reach these habitats for consumption by feeding larvae (Lacey and Inman (1985) J Am Mosq Control Assoc 1: 38-42); (Majori et al. (1987) J Am Mosq Control Assoc 3: 20-25); (Sundararaj and Rao (1993) Southeast Asian J Trop Med Public Health 24: 363-368); and (Osborn et al. (2007) Mem Inst Oswaldo Cruz 102: 69-72.) However, most of these formulations require several production steps including bacterial fermentation and purification and in some cases processing to add buoyancy, which are an expensive processes and do not include the use of B.t.Bs.

### Oil Bodies

Oil seed plants contain neutral lipids that are stored within the seed in subcellular organelles termed oil bodies, which serve as a source of energy to the germinating seedling. The oil bodies are coated with proteins that are specifically targeted to oil bodies, known as oil body proteins FIG. 1. The most abundant class of oil body proteins present in oil bodies, are called oleosins.

There is a great need for the identification of methods and materials that can be used to control insect pests. It is thus an objective of the present invention to provide new treatments and methods. The objectives are solved by the present invention.

### BRIEF SUMMARY OF THE INVENTION

The present invention is directed towards a method of pest control and provides easily administered insecticidal proteins derived from plants containing recombinant proteins and peptides that are functionally active and effective against many orders of insects.

In one embodiment, the present invention provides a plant flour material comprising a ground, dried plant material, wherein the plant material contains a recombinant protein comprises a bactericidal or an insecticidal protein toxin, or combinations thereof.

The bactericidal toxin can be Bt or Bti and the plant flour material can optionally contain BtB.

In one embodiment of the present invention the plant flour material composition comprises a mixture of at least three flours, wherein a first flour contains a Bt toxin, a second flour contains a BtB, and a third flour contains an insecticidal protein. In certain embodiments, the composition comprises at least two Bt flours, at least one BtB flour, and at least one flour containing a PRAP protein.

In certain embodiments, the present invention provides a seed oil body composition comprising a seed oil body fusion, said oil body fusion comprising a oil body protein fused to a recombinant protein comprising a bactericidal or an insecticidal protein toxin.

Other embodiments of the present invention provide methods of abating or controlling the population of mosquitoes comprising administering to a water source suspected of containing mosquito larvae an effective amount of seed oil body preparation comprising one or more Bti toxins, optionally in combination with one or more BtBs.

Another method provides methods of abating or controlling a pest insect population comprising administering to the pest insect population a food source containing two or more recombinant proteins comprising a bactericidal or an insecticidal protein toxin, or combinations thereof.

The invention further provides methods of controlling or abating a pathogenic microbial population comprising administering to an insect population, which serves as a host for the pathogenic microbial population, a recombinant plant material containing a recombinant protein comprising an bactericidal protein toxin.

### BRIEF DESCRIPTION OF THE DRAWINGS/FIGURES

The accompanying drawings, which are incorporated herein and form a part of the specification, illustrate the present invention and, together with the description, further serve to explain the principles of the invention and to enable a person skilled in the pertinent art to make and use the invention.

FIG. 1 depicts seed oil bodies surrounded by a protein rich phospholipids unit membrane and embedded in a protein matrix.

FIG. 2 provides the sequence of B.t.-toxin expression construct A2pt::Cry1Ac (3737 bp) and translation of toxin protein (SEQ ID NO: 1.)

FIG. 3 provides the sequence of B.t.Booster expression construct A2pt::CR9-MPED (3383 bp) and translation of B.t.Booster protein (SEQ ID NO: 2.)

FIG. 4 illustrates the expression of Cry1Ac and CR9-MPED mRNAs in A2pt::Cry1Ac/A2pt::CR9-MPED co-transformed Arabidopsis (First 10 lines): Expression levels, determined by qRT-PCR are shown relative to levels of actin ACT2 mRNA levels (i.e., delta CT or dCT value of 0.8 for Cry1Ac (line c/c#3) suggests the amount of CR9-MPED mRNA is 80% of actin).

FIG. 5 provides B.t. Enhanced killing of cabbage looper larvae with addition of B.t.Booster flour after applying a B.t.-flour prepared from transgenic Arabidopsis. Diet surface overlay bioassay with neonate Trichoplusia ni.

FIG. 6a provides the B.t.-enhanced killing of corn earworm larvae by the addition of B.t.Booster in a diet overlay bioassay after applying B.t.-flours prepared from transgenic Arabidopsis.

FIG. 6b provides surviving larvae weight data averaged from each group from the B.t.-enhanced killing of corn earworm larvae with the addition of B.t.Booster in a diet overlay bioassay after applying B.t.-flours prepared from transgenic Arabidopsis.

FIG. 7 provides a dose response curve illustrate the high toxicity of B.t.i-related Cry4Ba to the larvae of two mosquito species relative to untreated larvae (0 ng). Cry4Ba-GAV has 100-times more activity than Cry4Ba.

FIG. 8 shows that BtBs derived from mosquito receptor proteins enhance the toxicity of Bti Cry4Ba to Aedes aegypti larvae over mortality caused by Cry4Ba alone.

FIG. 9 shows fractionation of buoyant oil bodies from homogenized B. napus seeds. 200 mg of seeds homogenized in 4 ml of buffer and centrifuged for 10 min at 3000×g.

FIG. 10 provides B. napus oil bodies photographed under DIC (Differential Interference Contrast) microscopy.

FIG. 11 illustrates oleosin fusion vectors to deliver B.t.i and B.t.B to the seed oil body unit membrane.

FIG. 12 provides the sequence of an Oleosin GFP fusion expression construct A7pt::OL1-GFP (SEQ ID NO:3.)

FIG. 13 provides the sequence of Bti-toxin expression construct A7pt:OL1-Cry4Ba-GAV (SEQ ID NO: 4.)

FIG. 14 provides the sequence of BtBooster expression construct A7pt::OL1-AgCad (SEQ ID NO: 5.)

FIG. 15 provides A7pt::O-GFP expression in Arabidopsis produced the oleosin-GFP fusion protein in oil bodies. Fluorescent GFP (smaller concentric circles) was assayed in (A) seedling leaf, (B) seedling root, and (C) root hairs. Root hairs were co-stained with DAPI to demonstrate that GFP was in oil bodies, but not found in the similarly sized nuclei (larger objects.) GFP appears tightly tethered to oil bodies in various organs and cells and there was no detectable GFP fluorescence free in the cytoplasm.

FIG. 16, shows the levels of transgenic OL1:GFP mRNA in leaves relative to endogenous ACTIN7 mRNA levels set to equal 1 determined by qRT-PCR.

FIG. 17 provides the OL1-Cry4Ba mRNA levels in leaves of A7:OL1:Cry4Ba plant lines determined by qRT-PCR.

FIG. 18 provides the levels of transgenic OL1-AgCad mRNA in leaves relative to endogenous ACTIN7 mRNA levels.

FIG. 19 shows the expression of Cry4Ba or AgCad to OL1 in T2 seeds of transgenic plant lines determined by qRT-PCR.

FIG. 20 provides assay results for B.t.i toxin tethered plant oil bodies in a killing assay of Crulex quinquefasciatus larvae. Mortality was scored at 24 and 48 hours.

FIG. 21 depicts the protease cleavage site separting the oleosin from the protein of interest.

FIG. 22 depicts the vectors for expression of Formaecin Iₛ (FormIs) in plants.

FIG. 23 provides the sequence of a Formaecin Iₛ expression construct A7pt::FormIs (SEQ ID NO: 6.)

FIG. 24 depicts the leaf expression levels of A7pt::FormIₛ determined by qRT-PCR and normalized to ACTIN7 transcripts.

The present invention will be described with reference to the accompanying drawings. The drawing in which an element first appears is typically indicated by the leftmost digit(s) in the corresponding reference number.

### DETAILED DESCRIPTION OF THE INVENTION

The subject invention provides a method for controlling problem insect populations derived from plants containing insecticidal proteins. In one embodiment, the present invention employs the use of Bacillus thuringiensis (B.t.) and a B.t.Booster protein expressed in transgenic plants and the application of the insecticidal proteins in the form of a ground flour and oil body emulsions derived from transgenic plants. These proteins, however, are only exemplary. The present invention additionally provides plant flours and oils containing antimicrobial agents, such as PRAPs, and other useful proteins. As described herein, embodiments of the invention target insect pests inhabiting chicken houses and insect pests in aquatic environments. These objects can be accomplished by a variety of means that are known in the art, which are discussed in more detail below.

### Definitions

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

The term “chimeric” is used herein in the context of nucleic acid sequences refers to at least two linked nucleic acid sequences which are not naturally linked. Chimeric nucleic acid sequences include linked nucleic acid sequences of different natural origins. For example, a nucleic acid sequence constituting a plant promoter linked to a nucleic acid sequence encoding human amylase is considered chimeric. Chimeric nucleic acid sequences also may comprise nucleic acid sequences of the same natural origin, provided they are not naturally linked. For example a nucleic acid sequence constituting a promoter obtained from a particular cell-type may be linked to a nucleic acid sequence encoding a polypeptide obtained from that same cell type, but not normally linked to the nucleic acid sequence constituting the promoter. Chimeric nucleic acid sequences also include nucleic acid sequences comprising any naturally occurring nucleic acid sequence linked to any non-naturally occurring nucleic acid sequence.

“Conservatively modified variants” applies to both amino acid and nucleic acid sequences. “Amino acid variants” refers to amino acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated (e.g., naturally contiguous) sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. It is recognized that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a nucleic acid which encodes a polypeptide is implicit in a described sequence with respect to the expression product, but not with respect to actual probe sequences. As to amino acid sequences, it will be recognized that individual substitutions, deletions or additions to a nucleic acid, peptide, polypeptide, or protein sequence which alters, adds or deletes a single amino acid or a small percentage of amino acids in the encoded sequence is a “conservatively modified variant” including where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

“Expression” refers to the transcription and/or translation of an endogenous gene, ORF or portion thereof, or a transgene in plants. In addition, expression refers to the transcription and stable accumulation of sense (mRNA) or functional RNA. Expression may also refer to the production of protein.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length polypeptide or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns can contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. In addition to containing introns, genomic forms of a gene can also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region can contain regulatory sequences such as promoters and enhancers that control or influence the transcription of the gene. The 3′ flanking region can contain sequences that direct the termination of transcription, post transcriptional cleavage, and polyadenylation.

As used herein, the term “gene expression” refers to the process of converting genetic information encoded in a gene into RNA (e.g., mRNA, rRNA, tRNA, or snRNA) through “transcription” of the gene (e.g., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (e.g., RNA or protein), while “down-regulation” or “repression” refers to regulation that decrease production. Molecules (e.g., transcription factors) that are involved in up-regulation or down-regulation are often called “activators” and “repressors,” respectively.

The invention encompasses isolated or substantially purified nucleic acid or protein compositions. In the context of the present invention, an “isolated” or “purified” DNA molecule or an “isolated” or “purified” polypeptide is a DNA molecule or polypeptide that, by the hand of man, exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein.

The word “plant” refers to any plant, particularly to agronomically useful plants (e.g., seed plants), and “plant cell” is a structural and physiological unit of the plant, which comprises a cell wall but may also refer to a protoplast. The plant cell may be in form of an isolated single cell or a cultured cell, or as a part of higher organized unit such as, for example, a plant tissue, or a plant organ differentiated into a structure that is present at any stage of a plant's development. Such structures include one or more plant organs including, but are not limited to, fruit, shoot, stem, leaf, flower petal, etc. Preferably, the term “plant” includes whole plants, shoot vegetative organs/structures (e.g. leaves, stems and tubers), roots, flowers and floral organs/structures (e.g. bracts, sepals, petals, stamens, carpels, anthers and ovules), seeds (including embryo, endosperm, and seed coat) and fruits (the mature ovary), plant tissues (e.g. vascular tissue, ground tissue, and the like) and cells (e.g. guard cells, egg cells, trichomes and the like), and progeny of same.

The term “flour” as used herein includes any ground up plant material. The plant material may be seeds, flowers, leaves, branches, stems, roots, or combinations thereof. Thus, the term “flour” is intended to include any part of a plant that may be ground.

As used herein, the terms “polynucleotide” or “nucleic acid” refer to a polymer composed of a multiplicity of nucleotide units (ribonucleotide or deoxyribonucleotide or related structural variants) linked via phosphodiester bonds, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl 2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl 2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2 thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The terms “polypeptide,” “peptide,” “protein”, and “protein fragment” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Promoters may be derived in their entirety from a native gene, or be composed of different elements, derived from different promoters found in nature, or even be comprised of synthetic DNA segments. A promoter may also contain DNA sequences that are involved in the binding of protein factors which control the effectiveness of transcription initiation in response to physiological or developmental conditions. The promoters used in the DNA constructs of the present invention may be modified, if desired, to affect their control characteristics.

The term “recombinant” when used with reference to a cell, nucleic acid, protein or vector indicates that the cell, nucleic acid, protein or vector has been modified by the introduction of a heterologous nucleic acid or protein, the alteration of a native nucleic acid or protein, or that the cell is derived from a cell so modified. Thus, e.g., recombinant cells express genes that are not found within the native (non-recombinant) form of the cell or express native genes that are over expressed or otherwise abnormally expressed such as, for example, expressed as non-naturally occurring fragments or splice variants. By the term “recombinant nucleic acid” herein is meant nucleic acid, originally formed in vitro, in general, by the manipulation of nucleic acid, e.g., using polymerases and endonucleases, in a form not normally found in nature. In this manner, operable linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and introduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

A “transgene” refers to a gene that has been introduced into the genome by transformation and is stably maintained. Transgenes may include, for example, genes that are either heterologous or homologous to the genes of a particular plant to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but that is introduced by gene transfer.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms.”

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

As used in the present disclosure and claims, the singular forms “a,” “an,” and “the” include plural forms unless the context clearly dictates otherwise.

It is understood that wherever embodiments are described herein with the language “comprising,” otherwise analogous embodiments described in terms of “consisting of” and/or “consisting essentially of” are also provided.

The term “oleosin” as used herein means an oil body protein found in plants that comprises three domains: 1) an N-terminal domain; 2) a centrally located hydrophobic domain; and 3) a C-terminal domain. Nucleic acid sequences encoding oleosins are known to the art. These include for example the Arabidopsis oleosin (Van Rooijen et al. (1991) Plant Mol Bio 18: 1177-1179); the maize oleosin (Qu and Huang (1990) J Biol Chem 265: 2238-2243); rapeseed oleosin (Lee and Huang (1991) Plant Physiol 96: 1395-1397); and the carrot oleosin (Hatzopoulos et al. (1990) Plant Cell 2: 457-467.) There are many other seed oil body proteins in plants.

### Generation of Transgenic Plants

A large number of techniques are available for inserting DNA into a plant host cell. Transformation of plants with the genetic constructs disclosed herein can be accomplished using techniques well known to those skilled in the art. Those techniques include transformation with T-DNA using Agrobacterium tumefaciens or Agrobacterium rhizogenes as transformation agent, fusion, injection, biolistics (microparticle bombardment), or electroporation as well as other possible methods. If Agrobacteria are used for the transformation, the DNA to be inserted has to be cloned into special plasmids, namely either into an intermediate vector or into a binary vector. The intermediate vectors can be integrated into the Ti or Ri plasmid by homologous recombination owing to sequences that are homologous to sequences in the T-DNA. The Ti or Ri plasmid also comprises the vir region necessary for the transfer of the T-DNA. Intermediate vectors cannot replicate themselves in Agrobacteria. The intermediate vector can be transferred into Agrobacterium tumefaciens by means of a helper plasmid (conjugation). Binary vectors can replicate themselves both in E. coli and in Agrobacteria. They comprise a selection marker gene and a linker or polylinker which are framed by the right and left T-DNA border regions. They can be transformed directly into Agrobacteria (Holsters et al. (1978) Mol. Gen. Genet. 163: 181-187). The Agrobacterium used as host cell is to comprise a plasmid carrying a vir region. The vir region is necessary for the transfer of the T-DNA into the plant cell. The bacterium so transformed is used for the transformation of plant cells. Whole plants can then be regenerated from the infected plant material (for example, pieces of leaf, segments of stalk, roots, but also protoplasts or suspension-cultivated cells) in a suitable medium, which may contain antibiotics or biocides for selection. The plants so obtained can then be tested for the presence of the inserted DNA.

Ligation of the DNA sequence encoding the targeting sequence to the gene encoding the polypeptide of interest may take place in various ways including terminal fusions, internal fusions, and polymeric fusions. In all cases, the fusions are made to avoid disruption of the correct reading frame of the oil-body protein and to avoid inclusion of any translational stop signals in or near the junctions.

In many of the cases described, the ligation of the gene encoding the peptide preferably would include a linker encoding a protease target motif. This would permit the release of the peptide once extracted as a fusion protein. Additionally, for uses where the fusion protein contains a peptide hormone that is released upon ingestion, the protease recognition motifs may be chosen to reflect the specificity of gut proteases to simplify the release of the peptide.

A promoter is selected for its ability to direct the transformed plant cell's or transgenic plant's transcriptional activity to the coding region, to ensure efficient expression of the enzyme coding sequence to result in the production of insecticidal amounts of the subject protein, such as B. thuringiensis protein. Those skilled in the art will recognize that there are a number of promoters which are active in plant cells, and have been described in the literature. The particular promoter selected should be capable of causing sufficient expression of the enzyme coding sequence to result in the production of an effective insecticidal amount of the subject protein.

In addition, it may also be preferred to bring about expression of the subject protein, such as B. thuringiensis Δ-endotoxin, in specific tissues of the plant by using plant integrating vectors containing a tissue-specific promoter. Specific target tissues may include the leaf, stem, root, tuber, seed, fruit, etc., and the promoter chosen should have the desired tissue and developmental specificity. Therefore, promoter function should be optimized by selecting a promoter with the desired tissue expression capabilities and approximate promoter strength and selecting a transformant which produces the desired insecticidal activity in the target tissues.

In some preferred embodiments of the invention, genes encoding the bacterial toxin are expressed from transcriptional units inserted into the plant genome. Preferably, said transcriptional units are recombinant vectors capable of stable integration into the plant genome and enable selection of transformed plant lines expressing mRNA encoding the proteins.

In addition to numerous technologies for transforming plants, the type of tissue that is contacted with the foreign genes may vary as well. Such tissue would include but would not be limited to embryogenic tissue, callus tissue types I, II, and II, hypocotyl, meristem, root tissue, tissues for expression in phloem, and the like. Almost all plant tissues may be transformed during dedifferentiation using appropriate techniques described herein.

A variety of selectable markers can be used, if desired. Preference for a particular marker is at the discretion of the artisan, but any of the following selectable markers may be used along with any other gene not listed herein that could function as a selectable marker.

### B.t. and B.T.i Toxins

In certain embodiments, the present invention utilizes Bacillus thuringiensis crystal toxin genes that are insecticides of lepidopteran or coleopteran pests. In certain embodiments, the present invention utilizes Bacillus thuringiensis israelensis toxin genes, which are insecticides of dipteran pests.

It is understood that any B.t. toxin can be used in the present invention. B. thuringiensis forms crystals of proteinaceous insecticidal δ-endotoxins (Cry toxins) which are encoded by cry genes. Cry toxins have specific activities against species of the orders Lepidotera (Moths and Butterflies), Diptera (Flies and Mosquitoes) and Coleoptera (Beetles). More than 250 toxin-encoding genes have been isolated form B.t. collections. Among the endotoxins, the insecticidal crystalline proteins, called the delta-endotoxins, are suitable for use in the present invention. The names of the genes that encode the crystalline proteins are prefixed with ‘Cry’, as for example Cry1Ab, Cry1Ac, Cry9c, etc., and the proteins that are encoded by these genes are ‘Cry’ proteins.

Most of the B.t. toxins are insect group specific. Cry1Ac and Cry2Ab control the cotton bollworms, Cry1Ab controls corn borer, Cry3Ab controls Colorado potato beetle and Cry3Bb controls corn rootworm. These genes can be manipulated to alter the species activity of the B.t. toxin. Thus, for example, it is known that B.t.i toxin provides greater specificity for mosquito larvae than B.t. toxin.

It is understood that it is possible to modify the cry gene to yield a B.t. toxin that has the same or greater activity against mosquito larvae than B.t.i toxin. Thus, B.t. toxin and B.t.i toxin, as used herein, are intended to refer to the original genetic source of the toxin, and not necessarily its species selectivity.

Because of their specificity, these pesticides are regarded as environmentally friendly, with little or no effect on humans, wildlife, pollinators, and most other beneficial insects. B.t. toxin has been engineered to provide greater insecticidal activity. Such engineered B.t. toxins can be used in the present invention. Similarly, it is possible to reengineer a B.t. gene to provide a toxin that is more like B.t.i toxin, and vice versa.

In some preferred embodiments, B.t. or B.t.i peptide fragments can be used as the toxin for controlling insects. It is understood that site directed mutagenesis or related techniques can be used to identify the active portions of a protein, such that the transgenic protein incorporated into the fusions of the present invention are truncated or shortened forms of the wild-type gene. So long as the proteins maintain their efficacy against the target insect species, they remain suitable for purposes of the present invention. It is understood that though these mutations, the effectiveness of the protein can be either enhanced or partially impaired. A partial impairment of the efficacy of the protein is acceptable so long as the toxin protein remains capable of controlling the insect population of interest.

### BtB: Bt Booster

In certain embodiments, the present invention utilizes portions of cadherin proteins from the Bacillus thuringiensis protein toxin receptor also know as the B.t.Booster, which is known in the art. (U.S. Pat. Appl. 2005/0283857) and (Chen et al. (2007) Proc Natl Acad Sci USA 104: 13901-13906.)

The B.t.Booster when combined with B.t. toxins enhance the toxicity of B.t and causes the B.t. in some species not affected by its insecticidal properties to become susceptible to its insecticidal activity. The receptor used as the source of this domain(s) can be derived from various pests and insects, such as Manduca sexta, Heliothis virescens, Helicoverpa zea Spodoptera frugiperda and Plutella xylostella larvae. Many sequences of such receptors are publicly available. B.t.Booster peptide fragments can enhance a toxin's activity against the insect species that was the source of the receptor. It can also against enhance a toxin's activity against other insect species.

In specific embodiments, the invention relates to the use of a cadherin repeat 12-MPED peptide of Manduca sexta Bt-R₁ₐ cadherin-like protein to enhance the potency of B.t. toxins. Preferably, the cadherins can be Bacillus thuringiensis (B.t.) crystal protein (Cry) toxin receptors.

In one embodiment, the fragment of cadherin-like protein may be expressed as a fusion protein with a B.t. Cry toxin using techniques well known to those skilled in the art. As described herein, preferred fusions would be chimeric toxins produced by combining a toxin (including a fragment of a protoxin, for example) and a fragment of a cadherin-like protein.

In addition, mixtures and/or combinations of toxins and cadherin-like protein fragments can be used according to the subject invention.

### Antibacterial Toxins

Protein-based antibiotics have an advantage in that most may be produced from the product of a single gene. A variety of protein-based antibiotics can be used to kill coliform bacteria, such as pathogenic Salmonella and E. coli species. For example, the 522 amino acid long (a.a.) colicin E1 immunity protein produced by the ColE1 plasmid (Meagher et al. (1977) Cell 10: 521-536) and (Carraminana et al. (1997) Vet Microbiolo 54: 375-83), could be used in the present invention.

Proline-rich antibacterial peptides (PRAPs) are generally 15 to 21 a.a. in length and rich in proline residues. PRAPs can be produced in crop plants like rice or canola at an order of magnitude lower cost than by conventional methods of peptide synthesis such as organic chemistry or engineered microorganisms. PRAPs produced in crop plants offer an inexpensive approach to controlling pathogenic microbial populations, such as Salmonella. In poultry production facilities, for example, PRAP-flours can be applied as a flour to the litter. The PRAPs have no known toxicity in animals, and hence, meet an important safety requirement for the large-scale application of antibacterial flours in poultry houses.

When fed to litter beetles or similar pest insects, PRAPs kill the pathogenic bacterial population resident within the gastrointestinal tact of those insects. Thus, for example, PRAPs can be used to control S. enterica serotype Enteritidis, Salmonella, and E. coli populations.

TABLE 1
Proline-rich antibacterial peptides.
Protein Toxin Sequence

Formaecin I GRPNPVNNKPT*PHPRL
(natural)
Formaecin Is GRPNPVPNPKPPHPRL
(synthetic)
Apidaecin GNNRPVYIPQPRPPHPRI
(natural)
Drosocin
GKPRPYSPRPT*SHPRPIRV
(natural)
Pyrrhocoric in
VDKGSYLPRPT*PPRPIYNRN
(natural)
PRAP5,
Compound #5
GRPDKPRPYLPRPRPPRPVRL
(synthetic)
T* residues are glycosylated.

A number of synthetic consensus PRAP sequences have been reported to have improved antibacterial activities to Salmonella or other coliform bacteria (Otvos (2002) Cell Mol Life Sci. 59: 1138-1150); (Markossian et al. (2004) Biochemistry (Mosc) 69: 1082-1091; and (Kaur et al. (2007) Protein Sci 16: 309-315.) Among these are several PRAPs in which the glycosylated threonine residue has been replaced with another a.a. without loss in antibacterial activity (Kaur et al. (2007) Protein Sci. 16: 309-315.) Thus, the glycosylation of PRAPs is not important to their antibacterial activity.

Exemplary PRAPs that can be used in the present invention include those provided in Table 1, specifically including Formaecin Iₛ. Formaecin Iₛ has stronger antibacterial activity than formaecin I. PRAP5 is a consensus sequence derived from multiple PRAP sequences (Otvos et al. (2005) J. Med. Chem. 48: 5349-5359), which is incorporated by reference herein in its entirety. PRAP5 has been reported to be extremely effective against multidrug-resistant bacteria and in particular fuoroquinolone-resistant clinical isolates of E. coli and Klebsiella pneumoniae. PRAP5 has exceptionally strong antibacterial activity, killing the bacteria tested with an LD₅₀ of ˜5 μg/ml (˜2 μM). At a one thousand times higher concentration (˜2 mM), PRAP5 did not kill cultured Chinese Hamster Ovary cells.

Producing bactericidal proteins like formaecin Iₛ and PRAP5 in plant-based flours and applying them as a dust directly in chicken houses has a number of advantages in both safety and low cost over other methods of Salmonella control.

### Oil Body Fusions

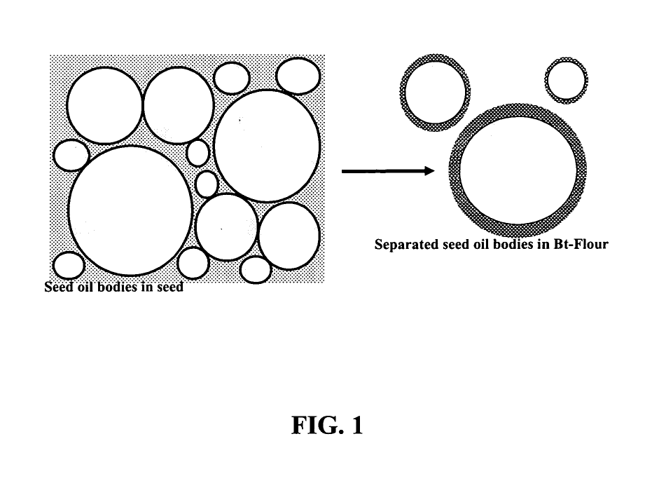

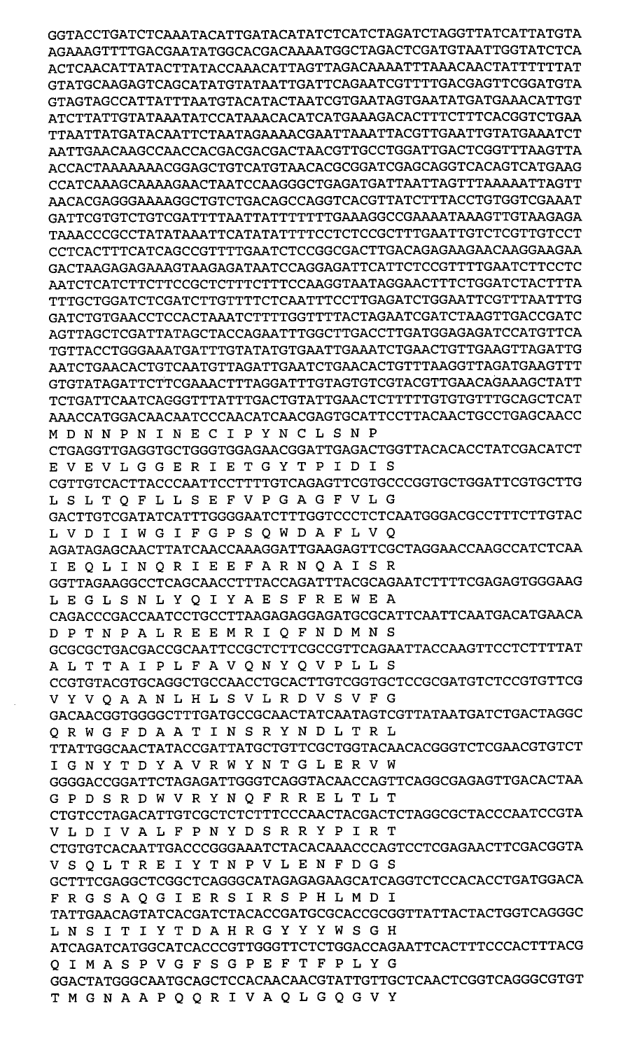

Oil bodies are small, spherical, subcellular organelles encapsulating stored triacylglycerides, an energy reserve used by many plants. Although they are found in most plants and in different tissues, they are particularly abundant in the seeds of oil seeds where they range in size from under one micron to a few microns in diameter. Oil bodies are comprised of the triacylglycerides surrounded by a half-unit membrane of phospholipids and embedded with a unique group of protein known as an oil body protein. See FIG. 1. The term “oil body” or “oil bodies” as used herein includes any or all of the triacylglyceride, phospholipid or protein components present in the complete structure.

In plants, the predominant oil body proteins are termed “oleosins”. Oleosins have been cloned and sequenced from many plant sources including corn, rapeseed, carrot and cotton. The oleosin protein appears to be comprised of three domains; the two ends of the protein, N- and C-termini, are largely hydrophilic and reside on the surface of the oil body exposed to the cytosol while the highly hydrophobic central core of the oleosin is firmly anchored within the membrane and triacylglyceride. Oleosins from different species represent a small family of proteins showing considerable amino acid sequence conservation, particularly in the central region of protein. Within an individual species, a small number of different isoforms may exist.

B.t.i. and B.t.Boosters can be tethered as fusions to oleosins through genetic modification and produced in plants. Production of recombinant proteins tethered to oleosins is inexpensive, because their attachment to this buoyant fraction is easily separated from 90% of the remaining total seed protein. The ground plant material containing the recombinant proteins is used to target the habitat of insects that can include the floor of chicken houses or water habitats of disease causing insects.

In a further embodiment of the invention, it is contemplated that proteins other than plant oleosins and proteins with homology to plant oleosins that may specifically associate with triglycerides, oils, lipids, fat bodies or any hydrophobic cellular inclusions in the host organism may be fused to a recombinant protein and used in the manner contemplated.

The coupling of target proteins, such as B.t.i.s and B.t.B.s, to oleosins enhances the efficient delivery of proteins to the environment, because the proteins are tightly coupled to oil bodies during preparation and remain semi-stably attached once delivered to the environment.

For water borne insects, such as mosquitoes, the oil bodies migrate at varying depths in aqueous environments allowing the insecticide to be consumed as the insect larvae filter feed. B.t.i. and/or B.t.Booster oil bodies that float at various levels in the water column can substantially improve control of disease-vectoring mosquito species by biological pesticides, lowering the amount of chemical pesticides entering aquatic environments and thereby producing significant public health-related benefits through better control of vector-borne diseases.

Oil bodies from individual plant species exhibit a roughly range of size and densities which is dependent in part upon the precise protein/phospholipid/triacylglyceride composition. But since the oil body is composed predominantly of oil the buoyant density of an oil body approximated that of the oil component. As a result, oil bodies may be simply and rapidly separated from liquids of different densities in which they are suspended. For example, in aqueous media where the density is greater than that of the oil bodies and approximates that of water, they will float under the influence of gravity or applied centrifugal force. Oil bodies may also be separated from liquids and other solids present in solutions or suspensions by methods that fractionate on the basis of size.

The oil bodies of the subject invention are preferably obtained from a seed plant and more preferably from the group of plant species comprising: thale cress (Arabidopsis thaliana), rapeseed (Brassica spp.), soybean (Glycine max), sunflower (Helianthus annuus), oil palm (Elaeis guineeis), cottonseed (Gossypium spp.), groundnut (Arachis hypogaea), coconut (Cocus nucifera), castor (Ricinus communis), safflower (Carthamus tinctorius), mustard (Brassica spp. and Sinapis alba), coriander (Coriandrum sativum) linseed/flax (Linum usitatissimum), Pittosporum species, and maize (Zea mays). Plants are grown and allowed to set seed using agricultural cultivation practices well known to a person skilled in the art.

Brassica napus (Canola) and Arabidopsis thaliana are both members of the plant family Brassicaceae (Cruciferae) and share a close common ancestry. Arabidopsis seeds are much smaller than those from Canola, but have homologous oil bodies and have a oil content of 39% to 45% (Jolivet et al. (2004) Plant Physiol Biochem. 41: 501-509.)

There are a number of oil rich seeds that might be a target for B.t.i and B.t.B expression that would help in the preparation of particulates for Anopheles control. These include soybean, peanut, sunflower, canola, corn, and flax. Canola (e.g., Brassica napus) is an edible crop in which the seeds are typically 40 to 43% oil. Canola is a big commercial crop in Canada and the Northern USA. Canola is an easily transformable plant species and is perhaps the best plant for field-scale application of this technology. In one embodiment, the B.t.i-Canola according to the present invention will be crop planted in extremely small acreage and well contained. The entire US market for B.t.i-related pesticides could be met with a thousand acres of well-contained B.t.-flour producing plants.

Canola seed flour and the flour from the seeds of other oils seed plants contain oil rich bodies that will float high in the water column for an extended period of time. Seed oil in a seed oil body is surrounded by a phospholipid half-unit membrane that is rich in lipophilic proteins that make up less than 5% of their total weight (Jolivet et al. (2004) Plant Physiol Biochem. 41: 501-509), and then by a protein-rich cytoplasmic matrix. B.t.B proteins can be engineered so that they are expressed in this matrix. Approximately 90% of the protein content is comprised of a small family of integral membrane proteins called oleosins. Steroleosin and caleosin are also proteins associated with the oil body but are much less abundant than oleosins. The oleosins are efficient carriers for delivering foreign fusion proteins to lipid bodies in B. napus (van Rooijen and Moloney (1995) Biotechnology (NY) 13: 72-77) and (Capuano et al. (2007) Biotechnol. Adv. 25: 203-206.) Oleosin-fusion proteins are quite stable. (van Rooijen and Moloney (1995) Biotechnology (NY) 13: 72-77) and (Capuano et al. (2007) Biotechnol. Adv. 25: 203-206) showed that 50% of the initial GUS enzyme activity of an oleosin-GUS fusion protein in Arabidopsis oil bodies persisted in the buoyant oil body fraction three weeks after oil body isolation. The production of recombinant proteins that are tethered to oleosins in oil-seed bodies should be inexpensive because their attachment to this buoyant fraction is easily separated from 90% of the remaining total seed protein. Hence, only a few more purification steps are required to purify the recombinant protein. The B.t.-flour concept is even simpler, because the homogenized oil bodies themselves are the product- easy to prepare and use directly to kill mosquito larvae. Thus, in one embodiment, engineering the covalent coupling of B.t.is and B.t.Bs to oleosins enhances the efficient delivery of mosquitocidal proteins to the environment, because the proteins should be tightly coupled to oil bodies during preparation and they should remain more stably attached once delivered to the environment. It may not be necessary to remove any protein bodies or seed coats to use oil body tethered B.t. products.

The use of a modified oleosin protein as a carrier or targeting means provides a simple mechanism to recover proteins. The chimeric protein associated with the oil body may be separated away from the bulk of cellular components in a single step by isolation of the oil body fraction using for example centrifugation size exclusion or floatation. The invention contemplates the use of heterologous proteins, including enzymes, therapeutic proteins, diagnostic proteins and the like fused to modified oleosins and associated with oil bodies. Association of the protein with the oil body allows subsequent recovery of the protein by simple means (centrifugation and floatation).

An insecticide protein-oil body protein fusion may also be prepared using recombinant DNA techniques. In such a case the DNA sequence encoding the insecticide and or receptor peptide is fused to a DNA sequence encoding the oil body protein, resulting in a chimeric DNA molecule that expresses a ligand-oil body protein fusion protein. In order to prepare a recombinant fusion protein, the sequence of the DNA encoding the insecticide must be known or obtainable. By obtainable it is meant that a DNA sequence sufficient to encode the protein may be deduced from the known amino acid sequence. It is not necessary that the entire gene sequence of the insecticide and or receptor peptide be used.

The present invention further provides a method for producing an altered seed meal by producing a heterologous polypeptide in association with a plant seed oil body fraction, said method comprising: a) introducing into a plant cell a chimeric DNA sequence comprising: 1) a first DNA sequence capable of regulating the transcription in said plant cell of 2) a second DNA sequence wherein said second sequence encodes a fusion polypeptide and comprises (i) a DNA sequence encoding a sufficient portion of an oil body protein gene to provide targeting of the fusion polypeptide to an oil body, linked in reading frame to (ii) a DNA sequence encoding the heterologous polypeptide and 3) a third DNA sequence encoding a termination region; b) regenerating a plant from said plant cell and growing said plant to produce seed whereby said heterologous polypeptide is expressed and associated with oil bodies; and c) crushing said seed and preparing an altered seed meal.

The present invention also provides a method of preparing an enzyme in a host cell in association with an oil body and releasing said enzyme from the oil body, said method comprising: a) transforming a host cell with a chimeric DNA sequence comprising: 1) a first DNA sequence capable of regulating the transcription of 2) a second DNA sequence, wherein said second sequence encodes a fusion polypeptide and comprises (i) a DNA sequence encoding a sufficient portion of an oil body protein gene to provide targeting of the fusion polypeptide to an oil body; (ii) a DNA sequence encoding an enzyme and (iii) a linker DNA sequence located between said DNA sequence (i) encoding the oil body and said DNA sequence (ii) encoding the enzyme and encoding an amino acid sequence that is cleavable by the enzyme encoded by the DNA sequence (ii); and 3) a third DNA sequence encoding a termination region functional in said host cell b) growing the host cell to produce the fusion polypeptide under conditions such that enzyme is not active; c) recovering the oil bodies containing the fusion polypeptide; and d) altering the environment of the oil bodies such that the enzyme is activated and cleaves itself from the fusion polypeptide.

To isolate the oil bodies, the plant material, such as the seed, can be ground. Seed grinding may be accomplished by any comminuting process resulting in a substantial disruption of the seed cell membrane and cell walls without compromising the structural integrity of the oil bodies present in the seed cell. Suitable grinding processes in this regard include mechanical pressing and milling of the seed. Wet milling processes such as described for cotton (Lawhon et al. (1977) J. Am. Oil Chem. Soc. 63: 533-534) and soybean (U.S. Pat. No. 3,971,856); (Carter et al. (1974) J. Am. Oil Chem. Soc. 51: 137-141) are particularly useful in this regard. Suitable milling equipment capable of industrial scale seed milling include colloid mills, disc mills, pin mills, orbital mills, IKA mills and industrial scale homogenizers. The selection of the milling equipment will depend on the seed, which is selected, as well as the throughput requirement.

It is understood that the process of milling will depend on the plant source. Certain plants and certain plant materials are more fibrous than other plant materials. The more fibrous the plant material will generally require a more vigorous or robust milling process. The times, conditions, and properties of milling to obtain a suitable milled material are generally known to those skilled in that art.

After harvesting the seed and removal of foreign material such as stones or seed hulls, for by example sieving, seeds are preferably dried and subsequently processed by mechanical pressing, grinding or crushing. The oil body homogenate can be used to kill insects without further purification or can be processed further. The oil body fraction may be obtained from the crushed seed fraction by capitalization on separation techniques which exploit differences in density between the oil body fraction and the aqueous fraction, such as centrifugation, or using size exclusion-based separation techniques, such as membrane filtration, or a combination of both of these. Typically, seeds are thoroughly ground in five volumes of a cold aqueous buffer or can be washed in distilled water. A wide variety of buffer compositions may be employed, provided that they do not contain high concentrations of strong organic solvents such as acetone or diethyl ether, as these solvents may disrupt the oil bodies.

Following grinding, the homogenate is centrifuged resulting in a pellet of particulate and insoluble matter, an aqueous phase containing soluble components of the seed, and a surface layer comprised of oil bodies with their associated proteins. The oil body layer is skimmed from the surface or otherwise isolated. The oil bodies can then be resuspended in one volume of fresh grinding buffer to increase the purity of the oil body fraction. Generally, aggregates of oil bodies are dissociated as thoroughly as possible in order to ensure efficient removal of contaminants in the subsequent washing steps. The resuspended oil body preparation is layered under a floatation solution of lower density (e.g. water, aqueous buffer) and centrifuged, again, separating oil body and aqueous phases. This washing procedure is typically repeated at least three times, after which the oil bodies are deemed to be sufficiently free of contaminating soluble proteins as determined by gel electrophoresis. It is not necessary to remove all of the aqueous phase and to the final preparation water or 50 mM Tris-HCl pH 7.5 may be added and if so desired the pH may be lowered to pH 2 or raised to pH 10. Protocols for isolating oil bodies from oil seeds are available in (Murphy, D. J. and Cummins I., (1989), Phytochemistry, 28: 2063-2069) and (Jacks, T. J. et al. (1990) JAOCS, 67: 353-361), the protocols of which are herein incorporated by reference in their entirety.

Oil bodies other than those derived from plants may also be used in the present invention. A system functionally equivalent to plant oil bodies and oleosins has been described in bacteria (Pieper-Fuirst et al. (1994) J. Bacteriol. 176: 4328), algae (Rossler, P. G. (1988) J. Physiol. (London) 24: 394-400) and fungi (Ting, J. T. et al. (1997) J. Biol Chem. 272: 3699-3706.) Oil bodies from these organisms, as well as those that may be discovered in other living cells by a person skilled in the art, can also be employed according to the subject invention.

A list of other compounds that might be produced by engineering plants to be expressed in or enriched in seeds and then prepared for delivery as oil body sprays would include: other insecticides, bactericides, herbicides, vitamins, elemental nutrients, pigments, cosmetic proteins, etc. Protein antibiotics like the colicins could be produced as protein-oil emulsions for therapeutic applications, say being applied directly to skin or sprayed onto fruit trees to prevent blight damage to fruit (Hancock and Chapple (1999) Antimicrob. Agents Chemother. 43: 1317-1323); (Debbia (2000) Recent Prog, Med. 91: 106-108); (Qiu et al. (2003) Nat, Biotechnol. 21: 1480-1485) and (Suzuki et al. (2006) J Orthop Res 24: 327-332.)

Although exemplified above using seeds, it is understood that oil bodies are present in most plant tissues. Therefore, in at least one embodiment of the present invention, the recombinant oil bodies are isolated from the entire plant, which can even be a juvenile plant that has not yet produced any seeds. In other embodiments, selected portions of the plants, such as those portions of the plant that contain the highest yields of oil bodies, are used to isolate the oil bodies of the present invention.

In accordance with further embodiments of the invention methods and compositions are provided for the release of recombinant proteins and peptides fused to oleosin proteins specifically associated with isolated oil body or reconstituted oil body fractions. Additional proteins associated with the oil body that can be fused the recombinant protein include caleosin and steroleosin and are not limited to oleosin. In one embodiment the expression cassette comprises a first DNA sequence capable of regulating the transcription of a second DNA sequence encoding a sufficient portion of an oil body protein gene such as oleosin to provide targeting to an oil body and fused to this second DNA sequence via a linker DNA sequence encoding a amino acid sequence cleavable by a specific protease a third DNA sequence encoding the protein or polypeptide of interest. The protein of interest can be cleaved from the isolated oil body fraction by the action of said specific protease.

In certain embodiments of the present invention, the seed oil body fusions of the present invention are coupled to protein bodies to make them denser. Protein bodies are organelles found in the same seed cells that produce oil bodies.

In other embodiments of the present invention, higher levels of toxins and co- toxins are expressed in seed protein bodies than is possible with oil bodies. By coupling the protein bodies to the oil bodies, the complex is rendered less buoyant than a comparable oil body. Persons skilled in the art know standard techniques to fuse protein bodies and oil bodies. These include genetic engineering techniques and post-expression coupling techniques. For example, oil bodies could be engineered to express a transgenic membrane protein that bound to the surface of a protein on protein bodies. Then dimmers or oligomers of oil bodies and protein bodies would form with various densities depending upon the ratio of the buoyant oil bodies and the dense protein bodies. Alternatively, if protein bodies expressed a transgenic membrane protein that bound to the surface of a protein on oil bodies then dimmers or oligomers of oil bodies and protein bodies would form with various densities. Hundreds of protein-protein interaction domains are known and some interact strongly enough to hold two cell organelles together. Examples of interacting proteins or protein domains that might be used to develop OB-PB interactions and fusions, include but are certainly not limited to the following: T-cell receptor (TCR) with MCH, leucine zipper domains with each other, CD26 with CD86, and ZZ domain derived from protein A of Staphylococcus aureus with the Fc domain of rabbit immunoglobulin G (IgG). These interacting domains would have to be expressed on the surface of oil bodies and protein bodies to produce the desired range of densities for the clusters of organelles.

In certain embodiments, it is preferable to tether oil bodies and protein bodies, in that protein bodies express high levels of protein toxins and co-toxins like Bts and BtBs, respectively, in seeds. Protein bodies are composed almost entirely of protein, while oil bodies express most of their protein in a narrow strip of membrane on their surface. Thus, higher total amount of toxin or co-toxin can be expressed in protein bodies.

Thus, in at least one embodiment, the oil body fusion of the present invention contains a first recombinant protein, such as Bt. Tethered to the oil body is a protein body containing a second recombinant protein, for example either a second Bt or a BtB.

It is understood in using the terminology of A fused to B that the two could be directly fused, such as AB, or that there could be a linker sequence connecting two proteins or peptides.

In at least certain embodiments, as illustrated above, more than one oil body can be tethered together. In such embodiments, it is possible to separately produce recombinant oil body fusions of different Bts, to thereby control the expression of each Bt protein in the oil body. In such embodiments, broader spectrum insecticidal compositions can be prepared. Of course, it is understood that one or more BtBs could be tethered to the recombinant oil body, either by way of a second recombinant oil body or through a recombinant protein body.

In at least certain embodiments, mixtures of recombinant oil bodies and/or protein bodies may be administered to the water column without otherwise tethering the recombinant bodies.

The oil body fusions of the present invention are generally stored in air-tight, dark colored (or light impervious) containers. It is understood that oils oxidize upon exposure to light and air. Oxidization leads to the degradation of the oil body fusions of the present invention. Under certain embodiments, the oil body fusions are stored under refrigerated conditions, though this is not required. Under refrigerated conditions, the oil body fusions of the present invention can be stable for at least 3, at least 6, at least 9 months, and for greater than a year. Under room temperature conditions and with or without the addition of protease inhibitors, the oil body fusions of the present invention are stable for at least a week, at least 2, at least 4, or at least 8 weeks.

### Recombinant Flours

In one embodiment, the present invention provides recombinant flours.

As exemplified in the B.t.-flour concept, recombinant flours provide a more efficacious production and delivery of protein-containing dusts. Agricultural dusts could be prepared from dried ground plant material and delivered as directly to control plant or animal pests or pathogenic microbial populations.

The present invention provides a method for producing an altered plant material by producing a heterologous polypeptide in association with a plant material, said method comprising: a) introducing into a plant cell a chimeric DNA sequence comprising: 1) a first DNA sequence capable of regulating the transcription in said plant cell of 2) a second DNA sequence wherein said second sequence encodes a fusion polypeptide and comprises a DNA sequence encoding the heterologous polypeptide and a DNA sequence encoding a protease cleavage site, and 3) a third DNA sequence encoding a termination region; b) regenerating a plant from said plant cell and growing said plant to produce material whereby said heterologous polypeptide is expressed; and c) crushing said plant material and preparing an altered plant meal.

The present invention provides a method for producing an altered seed meal by producing a heterologous polypeptide in association with a plant material, said method comprising: a) introducing into a plant cell a chimeric DNA sequence comprising: 1) a first DNA sequence capable of regulating the transcription in said plant cell of 2) a second DNA sequence wherein said second sequence encodes a fusion polypeptide and comprises a DNA sequence encoding the heterologous polypeptide and a DNA sequence encoding a protease cleavage site, and 3) a third DNA sequence encoding a termination region; b) regenerating a plant from said plant cell and growing said plant to produce seeds whereby said heterologous polypeptide is expressed; and c) crushing said seeds and preparing an altered seed meal.

The previously described techniques for isolating oil bodies generally apply to the manufacture of recombinant flours. Initially the plant material is dried and the dried plant material is ground into a powder.

It is understood that plant cells contain proteases proteases and the protein bodies will be most stable when left in intact seeds. In seeds the oil bodies should have half lives of several months to years. When plant material is ground, the proteases are released and they can negatively influence the shelf-life and activity levels of the target proteins. Therefore, in at least certain embodiments, the activity levels of the endogenous proteases are controlled or eliminated. For example, storing the flour material in a dried form will substantially reduce the activity levels of the endogenous proteases. Similarly, the endogenous proteases can be controlled through the addition of protease inhibitors.

In one embodiment the subject invention would be available as a freshly ground material from stored seed or other plant material. The recombinant protein flour can be processed on site for example, by the farmer himself, from dried seed material containing the recombinant protein and then applied as freshly ground flour.

The conventional techniques used to process powdered pharmaceutical excipients generally apply to the production of the recombinant flours of the present invention. Thus, it is preferable that the flours of the present invention do not cake or stick during manufacture or storage. The flours are generally stored under storage stable conditions for powdered materials. Thus, it is generally preferable to control the flour's exposure to moisture, either through the use of external desiccants or by storing the flours in an air- tight container. It is understood, as previously explained, that exposure to moisture can reactivate endogenous proteases within the flour material. This will lead to the degradation of the active protein of interest. Exposure to moisture can also cause the flour material to cake, which will interference with the subsequent application or use of the flour material. Persons skilled in the art understand how to control for exposure to moisture.

Under certain embodiments, the flours of the present invention are storage stable for 3, 6, 9 months, and up to a year or more.

It is understood that the flours of the present invention are generally milled to a size of at least about 100 microns or greater, or about 250 microns or greater, or about 500 microns or greater. In certain embodiments, the flours are ground to a size of about 2-8 mm in diameter. In selecting the size of the flour granules, consideration is generally given to the mandible size of the target insect population and its feeding habits.

As explained herein, in certain embodiments, the recombinant fours of the present invention are dispersed through broadcast spreaders. In certain applications, the recombinant flours can be hand spread or spread through a handheld broadcast spreader.

### Recombinant Protein Flours to Control Agricultural Pests

In one embodiment, the recombinant flours are directly applied to either the plant or environment as set forth in the methods of the present invention that follow. However, it is also understood that the recombinant flours of the present invention can also be processed into any of a number of forms.

Thus, for example, as described below, PRAP and Bt-containing flours alone or together or additionally in combination with BtB flour can be used as feed for beetles. In the setting of PRAP, the flour controls microbes present in the beetle's GI tract and in the case of Bt, the flour actually controls and reduces the beetle population. In certain embodiments, the recombinant flour can mixed with additional materials to create a food source for the beetles. Thus, for example, it is understood that beetles eat chicken feed. The recombinant flour can be added to the chicken feed and that chicken feed can be fed to the chickens. The infecting beetle population would then eat the feed that the chickens did not eat. Because PRAP and Bt are not toxic to humans or chickens, neither would be affected from such administration of the recombinant flour.

In other embodiments, the recombinant flour can be mixed with more traditional food sources directed to the beetles, food sources that the beetles have a greater affinity towards.

It is understood that the beetle food can contain PRAP to control the pathogenic microbes or BT to control the beetles themselves, or both. The beetle food could also contain BtB to enhance the effectiveness of the Bt. In certain embodiments, the BtB is expressed in the same flour as the Bt. In other embodiments, the Bt and BtB are expressed in different plants and separate flours are prepared. In at least certain embodiments, a first flour composition comprising a Bt protein and a second flour composition comprising a BtB protein are administered to the environment of the target insect, such as the litter beetle.

It is generally preferable to express the Bt in a first plant and the BtB in a second plant. Thus two separate flours can be prepared and combined. In so doing, it is easier to control for differences in expression levels between the two proteins. Of course, more than one Bt flour can be combined with more than one BtB flour. In so doing, it is possible to prepare a flour mixture with a broad spectrum of effectiveness. Thus, for example, one embodiment of the present invention provides a flour mixture with 2, 3, or more Bt's combined with 2, 3, or more BtBs. Added to such a mixture could be one or more PRAPs.

In preferred embodiments, the proteins are fed to target insects together with one or more insecticidal proteins, preferably (but not limited to) B.t. Cry proteins. When used in this manner, the peptide fragment can not only enhance the apparent toxin activity of the Cry protein against the insect species that was the source of the receptor but also against other insect species, in particular those lacking the corresponding receptor.

In certain embodiments, traditional pesticides can be added to the beetle food, generally at reduced concentrations. For example, traditional, organic insecticides including organophosphates, pyrethroids, spinosad, mylar, and boric acid can be added. In the context of a Bt or Bt and BtB containing flour-based food source, the beetles that ate that food would be stressed by reason of the ingestion of the Bt toxin. The addition of an additional toxin, at a reduced concentration to what was previously considered to be an effective dosage, is generally enough to kill the insect that ate that food source. By coupling the toxicity of a Bt with the toxicity of a pesticide, it is possible to reduce the usage of pesticides.

In certain embodiments, the recombinant protein flours are applied uniformly to the floor of poultry the house at a rate of 1-2 lbs for each 100 square feet, generally in bands along feeder lines. The recombinant flour material should be reapplied after each grow-out or after the addition of new litter material. In cases where reinfestation occurs or when very large populations remain active, retreatment is desirable after 2 to 3 weeks. In another embodiment, the recombinant protein flour can be delivered by combining it with the chicken feed.

It is understood that litter beetles crawl on top of the litter as they are make their way to feed line areas. Therefore, it is preferable to administer the recombinant flour material directly on top of the chicken litter. Application directly to the bare floor will generally not provide as good of results.

Although exemplified in the context of beetles, it is understood that similar approaches can be used to control other insect species.

In certain embodiments, the flours of the present invention are applied to crops to control pest insect infestations. Generally, in such embodiments, flour mixtures, containing multiple Bts, BtBs and/or PRAPs are used. Generally, the flours are mixed with additional excipients. For example, additional excipients may be added to provide bulk to the recombinant protein flours of the present invention. Bulking agents, such as microcrystaline cellulose, provide weight and prevent the flours from being unnecessarily spread by the wind, for example. Lipophilic compounds can be incorporated into the dry product, for example lecithins can be added to reduce the dispersion of fine particles when disturbed by air movement or when containers are opened. Tackyfying agents can be added to assist in the recombinant flours to adhere to the crops. Tackifying agents, such as resins, can be added at the time of manufacture of the recombinant flours or they can be added at the time of application to the crops. It is preferable that the tackifying agents used in these embodiments of the present invention are not water soluble and that they do not contain endogenous proteases. It is further preferable that the addition of the tackifying agent does not activate the endogenous proteases of the recombinant flours.

Antioxidant agents, such as BHA (butylated hydroxyanisol) or ethoxyquin (1,2 dihydro-6-ethoxy-2,2,4-trimethyquinole) can be added as preservatives to prevent the oxidation of oil contained in the flour mixture and to stabilize the mixture.

In certain embodiments, the recombinant protein flours are applied with fertilizer at a rate of 1-2 lbs for each 100 square feet. The recombinant flour material should be reapplied after each heavy rainfall. In cases where reinfestation occurs or when very large populations remain active, retreatment is desirable after 2 to 3 weeks.

In certain embodiments the recombinant protein flour is manufactured, produced and applied by one entity. Although one entity can facilitate the manufacture, production and application of the protein recombinant flour the subject invention can be achieved separately in components through the use of different actors.

### Oil Body Fusions to Control Agricultural Pests

Agricultural sprays, in particular, might be prepared from seeds and delivered as “seed oil-body sprays”. The hydrophobic nature of the seed oil body surfaces acts to emulsify the oil bodies and the proteins they carry into aqueous solution, but the oil in the bodies would act as a wetting agent once delivered and dried. Proteins bound in the matrix surrounding the seed oil bodies or proteins tethered to the seed oil body surface via links to oleosins or other oil body proteins would remain in solution emulsified with the oil bodies. For example, Bacillus thuringiensis toxins like the B.t. Cry1Ac that kill moth larvae and B.t.-Boosters like CR9-MPED (InsectiGen Products) that enhance their insecticidal activity could be modified to be expressed in association with or covalently attached to the oil seed bodies. The proteins would be produced inexpensively in a seed oil crop like canola and prepared at minimal expense by homogenization or milling into a B.t.-flour. These products would then be sprayed on plants to protect those plants from B.t./B.t.B sensitive insects like moth larvae. Again the seed oil body helps to hold the protein reagent in solution, but the oil in these bodies would act as a wetting agent helping to link the B.t./B.t.B preparation to the waxy surfaces of leaves.

The preceding sections discussed how the recombinant flours of the present invention can be used to control beetle populations, either by killing them or by controlling the pathogenic bacteria present in the GI tract of the beetles. Because beetles have a known affinity for moist places and moisture, in at least certain embodiments of the present invention the oil body fusion technology of the present invention is used to control beetle populations. In such embodiments, suitable PRAP and/or Bt oil body fusion constructs are made. Plants expressing such fusions can be used to manufacture suitable oil body fusions.

The oil body fusions containing PRAP and/or a Bt specific for beetle populations can then be used to formulate suitable food sources for the beetles. For example, moist traps containing paste-like food containing the PRAP and/or a Bt oil body fusions can be made. The beetles, with their preference for moist habitats, would be preferentially drawn to such food sources. Such oil body fusion-based foods are expected to demonstrate greater protease stability than a comparable flour-based food source to which water or some other liquid source has been added.

Oil Body Fusions to Control Water-Borne Insets

B.t.i and B.t.B could be expressed efficiently in, for example, canola seeds and milled into a B.t.i flour with a particle size and buoyancy approximating that of the food consumed by filter feeding mosquito larvae. Mosquito larvae will take up the buoyant cell-sized B.t.i-containing oil bodies, while feeding on the B.t.-flour, process the toxins, and die. This method of production and delivery will be much less expensive than current approaches. The wide range in buoyant densities of B.t.i-containing particles produced in a heterogeneous B.t.i-flour should reach all levels of the water column. By combining B.t.i and B.t.B together there will be stronger killing activity against larvae from a wider range of mosquito species. With the toxins and proteins tightly coupled to oil bodies, they should remain stably attached once delivered to the environment. The present invention can be used to modify any oleosin of interest, including any plant oleosin such as an Arabidopsis thaliana oleosin, a Brassica oleosin, or a corn oleosin.

In one embodiment, the subject invention provides a recombinant protein flour for mosquito control consisting of a diameter that is able to be consumed by the mosquito larvae. The diet of the mosquito consists of bacterial, protist and protist algal cells that have a diameter of 0.45 mm to 100 mm (Merritt et al. (1992) Annu. Rev. Entomol. 37: 348-376.) Mosquitoes can also accommodate larger particles of 1 mm diameter by chewing. The subject invention can be formulated during production to accommodate the particle size necessary to facilitate mosquito ingestion.

The subject method includes the steps of (a) preparing an expression cassette comprising: (1) a first nucleic acid sequence capable of regulating the transcription of (2) a second nucleic acid sequence encoding a sufficient portion of a mutant oleosin polypeptide to provide targeting to an oil body fused to (3) a third nucleic acid sequence encoding the heterologous polypeptide of interest; (b) delivering of the expression cassette into a host cell; (c) producing a transformed organism or cell population in which the chimeric gene product is expressed and (d) recovering the chimeric gene protein product through specific association with an oil body. The heterologous peptide is generally a foreign polypeptide normally not expressed in the host cell or found in association with the oil body.

The subject method includes the steps of (a) preparing an expression cassette comprising: (1) a first nucleic acid sequence capable of regulating the transcription of (2) a second nucleic acid sequence encoding a sufficient portion of a mutant oleosin polypeptide to provide targeting to an oil body fused to (3) a third nucleic acid sequence encoding the heterologous polypeptide of interest; (b) delivering of the expression cassette into a host cell; (c) producing a transformed organism or cell population in which the chimeric gene product is expressed and (d) recovering the chimeric gene protein product through specific association with an oil body. The heterologous peptide is generally a foreign polypeptide normally not expressed in the host cell or found in association with the oil body.

In an alternate embodiment of the present invention, a related B.t.i family of toxins kill the larvae from diverse mosquito species that inhabit different depths in shallow aquatic ecosystems. The B.t.i bound to oil bodies will float at different depths, be consumed by these larvae, and kill them.

One embodiment of the present invention provides B.t.i-related proteins and/or B.t.Bs produced in tight association with the buoyant oil bodies of an oil-rich plant seed plant. One embodiment of the present invention is where B.t.i and B.t.B expressed in seed oil bodies of oil-rich crop plants can be delivered effectively as a milled B.t.i-flour that will float at various levels in the water column and kill feeding mosquito larvae. B.t.i and B.t.B could be expressed efficiently, for example, in Canola seeds, and milled into a B.t.-flour with a particle size and buoyancy approximating that of the food consumed by filter feeding mosquitoes. The mosquito larvae will take up the buoyant B.t.i-flour, process the toxins, and die. This method of production and delivery will be much less expensive than current approaches. The range in densities of B.t.i-containing particles produced in a heterogeneous B.t.-flour should reach all levels of the water column. By combining B.t.i and B.t.B together there should be stronger killing activity toward a wider range of mosquito species larvae.

One embodiment of the present invention involves expression and/or co-expression of B.t.i Cry4Ba-GAV and B.t.B AgCad in the protein matrix surrounding the seed oil bodies of an oil rich model plant such as, Arabidopsis.

The buoyancy of oil bodies and stability of the B.t.i coupled to oil bodies can be optimized through techniques known to those skilled in the art. In certain embodiments, the recombinant oil bodies of the present invention have a buoyant density of about 0.8 g per mL to about 1.1 g per mL.

In these embodiments, the buoyant density of the oil body fusion material is controlled such that the oil body fusions are suspended in the proper location in the water column, depending on the target insect species. Therefore, in at least certain embodiments, substantially pure oil body emulsions are applied. However, it is understood that substantially pure oil body emulsions from some plant species may float too high within the water column to be effective. For such species, as explained elsewhere herein, steps can be taken to affect the buoyant density of the oil body emulsion. For example, coupling protein bodies to the oil bodies can affect the density of the fusions. Similarly, more coarsely ground plant material will have more protein material associated with the oil bodies and such oil bodies will settle lower in the water column.

In spite of B.t.i's high level of insect larval specificity and toxicity, insects with low receptor levels are naturally less susceptible to the toxicity of B.t.is. When portions of these receptor B.t.Booster proteins are added along with the appropriate B.t.i-related toxin, B.t.i toxicity can be enhanced 10-fold and the insect host range can be expanded to the larvae of less susceptible mosquito species. For example, the B.t.B AgCad is a peptide derived from a mosquito gut cadherin that binds Cry4Ba. AgCad enhances the killing activity of the B.t.i-produced Cry4Ba several fold for a number of mosquito larvae and extends the utility of Cry4Ba and the related Cry4Ba-GAV to a wider range of mosquito species (Hua et al. (2008) Biochem. 47: 5101-5110.) Furthermore, B.t.Boosters have no inherent toxicity of their own to mosquitoes or other animal species.

In one embodiment the invention is directed against mosquitoes that breed in permanent or semi-permanent, natural or artificial, aquatic habitats. Mosquitoes of major importance to be controlled by the present invention are species of the genera of Aedes, Anopheles, Culex, Culiseta, Coquillettidia, Deinocerites, Manosonia, Psorophora, Uranotaenia, and Wyeomyia. It is an objective of this invention to direct the use of the insecticidal delivery composition for the control of the immature aquatic stages of various species of mosquitoes before they become biting adults capable of being a nuisance and/or transmitting a disease. This technique is cost-effective and reduces the environmental and health hazards that can result when insecticides are extensively broadcast over large areas for the control of the adult stages.

In addition to mosquitoes, other species of aquatic environment insects such as biting and nonbiting midges, black flies, moth flies, crane flies, horse flies, deer flies, hover or flower flies can constitute a nuisance and often a health threat to humans and livestock. Thus, their growth as a population, if unchecked, can be detrimental. The medical and veterinary importance of various species of mosquitoes and other important aquatic environment insects are discussed in detail by Robert F. Harwood and Maurice T. James in “Entomology In Human and Animal Health,” Seventh Edition, 1979, MacMillan Publishing Co., Inc., New York, N.Y., which is incorporated herein by reference. Therefore, the scope of the present invention also relates to the use of the insecticidal delivery composition with one or more active insecticidal ingredients for controlling various species of aquatic environment insects other than mosquitoes.

It is also an object of the present invention to provide a composition and method which is easy to prepare (formulate) and use (apply), and which is safe to the environment, but which is effective for use in controlling one or more immature stages of natural population of aquatic environment insects, particularly mosquitoes.

In accordance with the present invention, there is provided an insecticidal delivery composition for controlling a population of aquatic environment insects which includes at least one B.t. insecticidal protein, and at least one different insecticidal B.t.B. agent which is being present in a total amount effective to control the population of aquatic environment insects.

Insect population is used here to refer to one or more groups or species of aquatic environment insects that breed in any type of aquatic environment or habitat requiring control treatment. The population as used herein denotes a natural or artificial breeding area and the like or the aquatic insects, pupae, larvae and eggs contained within any geographical area needing aquatic environment insect control treatment. For example, a field, yard, pasture, pot hole, salt marsh, ditch, tire, woods, lake, stream, river, bay, pond, etc., may be treated. Of course, the area needing aquatic environment insect control treatment can be any size and the present invention is only limited by the amount of time, equipment, and material available.

In general, the present invention is considered successful when it kills 95% of population. It is understood that complete lethality, therefore, is not required for the present invention to be useful and/or effective. The ultimate preferred goal is to prevent insects from damaging plants and/or transmitting pathogens. Thus, prevention of feeding is sufficient. Thus “inhibiting” the insects is all that is required. This can be accomplished by making the insects “sick” or by otherwise inhibiting (including killing) them so that protection is provided. Peptides of the subject invention can be used alone or in combination with another toxin to achieve this inhibitory effect, which can be referred to as “toxin activity.” Thus, the inhibitory function of the subject peptides can be achieved by any mechanism of action, directly or indirectly related to the Cry protein, or completely independent of the Cry protein.

Because of the novel approach, the subject invention offers new alternatives for pest control. The subject invention can be used to enhance and expand the spectrum (or insect range) of toxicity of a given insect-toxic protein. Based on the subject disclosure, one skilled in the art can practice various aspects of the subject invention in a variety of ways.

All patents and publications cited herein are fully incorporated by reference herein in their entirety.

### EXAMPLES

### Example 1

### B.t. and B.t.B Expression and Activity in Plants

It has been reported that spraying recombinant B.t.s and B.t.Bs on plants is highly effective at killing feeding moth larvae. It has been determined that B.t. Cry1Ac has improved codon usage for better plant expression and reduced proteolytic cleavage for greater protein stability in the insect gut.

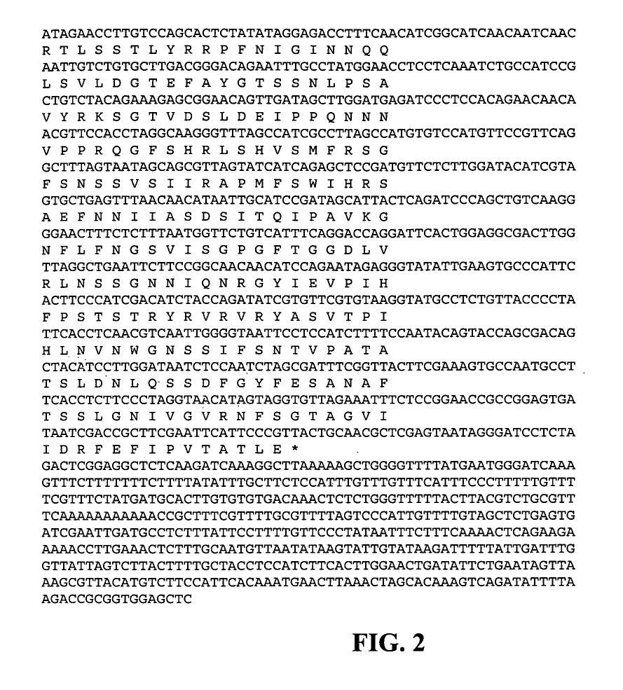

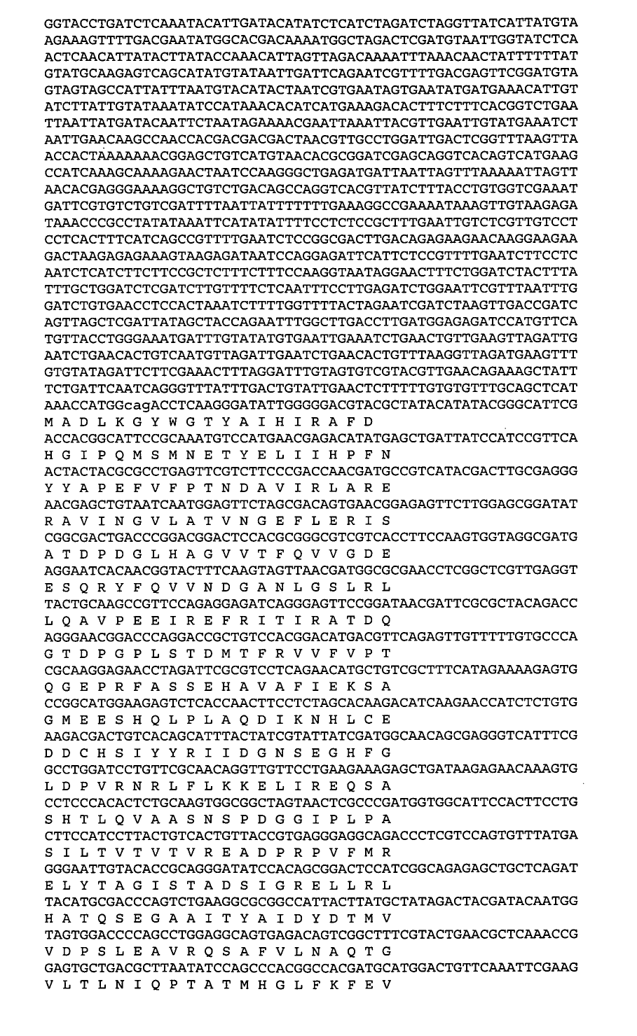

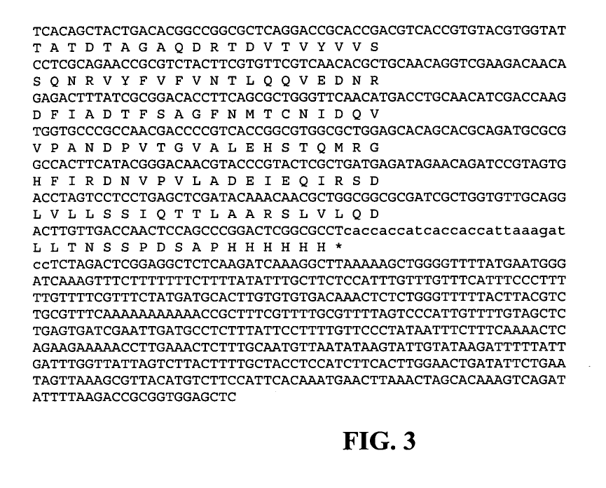

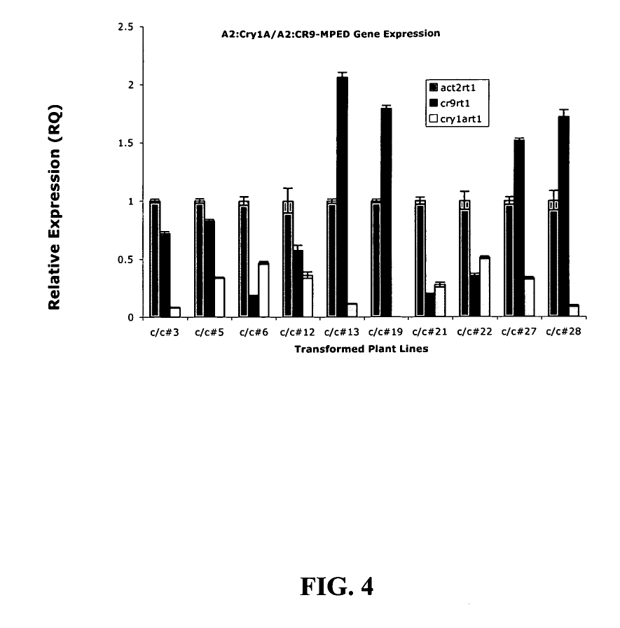

Transgenic plants co-expressing moth-larvae specific B.t.s and different B.t.Bs have been prepared. For example, B.t. Cry1Ac and B.t.B CR9-MPED cDNA sequences were cloned separately under the control of constitutive plant actin 2 promoter in two different A2pt expression cassettes with distinct linked resistance genes. Two transgenes, A2pt::Cry1A (BarR) (SEQ ID NO: 1; FIG. 2), and A2pt::B.t.B CR9-MPED (HygR) (SEQ ID NO: 2, FIG. 3) were transformed individually into Arabidopsis thaliana and these B.t. and B.t.B constructs were also co-transformed together into another set of plants. Ten to 20 T1 generation transgenic plant lines were generated for each of the three transgenic genotypes. Each line was quantified for expression of B.t. and B.t.B mRNA levels using quantitative Real-Time PCR and multiple primer pairs for each transcript. In summary, a wide range of B.t. and B.t.B mRNA expression levels were demonstrated among the 40+ fertile transgenic plants assayed. For example, FIG. 4 shows the levels of Cry1Ac and Cr9-MPED mRNA in 10 co-expressing lines normalized to endogenous actin ACT2 mRNA levels. Table 2 lists a summary of relative expression levels for noteworthy plants of the three genotypes. These plants meet the particular needs of experiments determining the enhanced mortality resulting from the coexpression of B.t.B CR9-MPED with B.t. Cry1Ac.

TABLE 2
Example plants with useful relative quantities (RQ)
of Cry1Ac and CR9-MPED mRNAs.

| | RQ ratios for |
|---|---|
| Transgenic plant line | ACT2:Cry1Ac:CR9-MPED (1:x:y) |
| (B.t. Cry1Ac) Cry1 #31 | 1:0.20:0 |
| (B.t. Cry1Ac) Cry1 #1 | 1:0.35:0 |
| (B.t. Cry1Ac) Cry1 #3 | 1:0.75:0 |
| (B.t. Cry1Ac) Cry1 # | 1:1.25:0 |
| (CR9-MPED) Cr9 #2 | 1:0:1.0 |
| (CR9-MPED) Cr9 #4 | 1:0:1.6 |
| (CR9-MPED) Cr9 #1 | 1:0:2.0 |
| (B.t. + CR9-MPED) c/c #3 | 1:0.1:0.75 |
| (B.t. + CR9-MPED) c/c #27 | 1:0.3:1.4 |
| (B.t. + CR9-MPED) c/c #13 | 1:0.1:2.0 |

^{a}RQ values were normalized to ACT2 (A2) (internal control) mRNA, which was set to one. ACT2 mRNA is estimated to be 0.05 to 0.1% of total mRNA.

Endogenous actin ACT2 levels were measured and set to 1 in each plant and used to normalize all expression of Cry1Ac and CR9-MPED mRNA levels. The ratios of ACT2 to Cry1Ac to CR9-MPED mRNAs are listed in column 2 of Table 2 in order that simple comparisons can be made among these example lines.

The mortality of moth larvae species was assayed examining the feeding of the three genotypes of transgenic plants relative to each other and relative to wild-type plant controls. An initial result indicates extremely high expression levels of the B.t. and B.t.B expressed from the ACTIN 2 A2pt cassette can be achieved. B.t. levels were so high that many of the standard insects (Heliothis zea, corn earworm) at several instar stages used in traditional B.t. spray and B.t.+B.t.B co-spray experiments were killed out right on the plants with the lowest levels of B.t. Cry1Ac (plant line Cry1#31, Table 2). The mortality of Fall Army Worm, Spodoptera frupperda, which is 100-fold less sensitive to Cry1Ac, was assayed feeding on these plants. Killing of the Fall Army Worm was greatly enhanced by co-expressing B.t.B CR9-MPED along with B.t. Cry1Ac, while B.t. alone has only weak killing activity and CR9-MPED causes no increased mortality over control plants.

### Example 2

### Co-Application of Plant-Based Bt-Flours with Bt-Toxin and Bt-Receptor Proteins Enhanced Insect Toxicity

Biologically active Bt-flours were prepared directly by drying and grinding transgenic plant material. Bt-flours were prepared from the dried shoots of plants expressing Cry1Ac, CR9-MPED, and CR12134 alone.

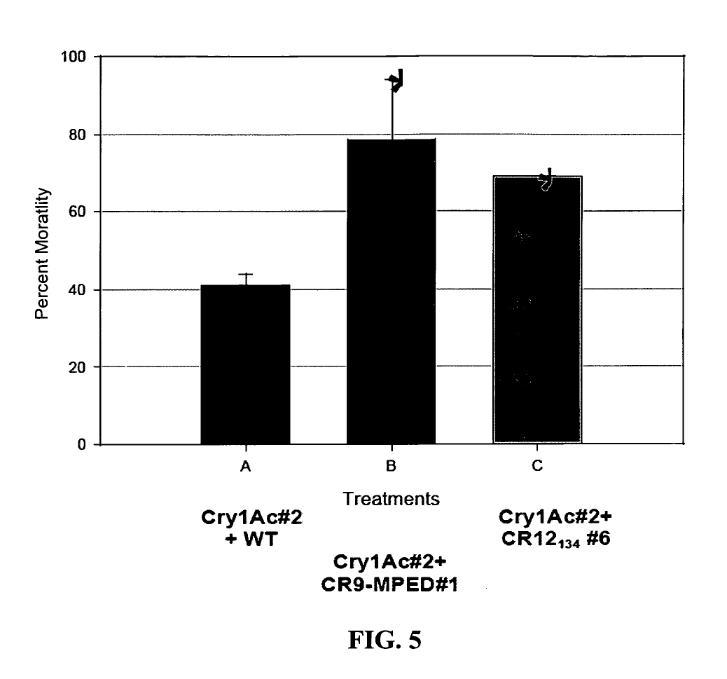

Diet bioassay trays were loaded with approx. 1.0 ml multispecies diet prepared according to manufacturer's directions (Southland Products Inc., Lake Village, Ark.) and allowed to come to room temperature. One hundred microliters of a suspension of Bt-flour prepared from plants expressing Cry1Ac and equal quantities of flour prepared from either wild-type plants or from plants expressing CR9-MPED or CR12134 was loaded into wells (16 wells/replicate). Individual cabbage looper (Trichoplusia ni) neonates were transferred to each well and covered with tray covers, and insect mortality was scored after 5 days (FIG. 5).

In FIG. 5, sample size: 16 larvae/rep x 2 rep/treatment. Bioassay was scored on day 5. Flour from transgenic Cry1Ac#2-Arabidopsis was mixed with equal mass of wild type (wt), transgenic CR9-MPED#1, or transgenic CR12134#6 plant powder. Controls: 0-5% mortality was observed in treatments with 0.2mg/cm2 CR9-MPED#1, 0.2mg/cm2 CR12134#6, or 0.2 mg/cm2 wild type. *Significant enhancement was observed for both CR9-MPED#1 and CR12134#6.

The legend for FIG. 5 is A. 0.1 mg/cm2 (dw) Cry1Ac#2-Arab+0.1 mg/cm2 wild type Arabidopsis; B. 0.1 mg/cm2 (dw) Cry1Ac#2-Arab.+0.1 mg/cm2 CR9-MPED#1; C. 0.1 mg/cm2 (dw) Cry1Ac#2-Arab.+0.1 mg/cm2 CR12134#6.

Flours prepared from both CR9-MPED and CR12134 proteins significantly enhanced the mortality of Bt-flour from Cry1Ac plants. Flours prepared from wild-type plants and from plants expressing CR9-MPEP and CR12134 alone did not cause any significant insect mortality (data not shown). This study clearly shows the potential for using Bt-flours prepared from ground plant material. However, cabbage looper is susceptible to Bt alone.

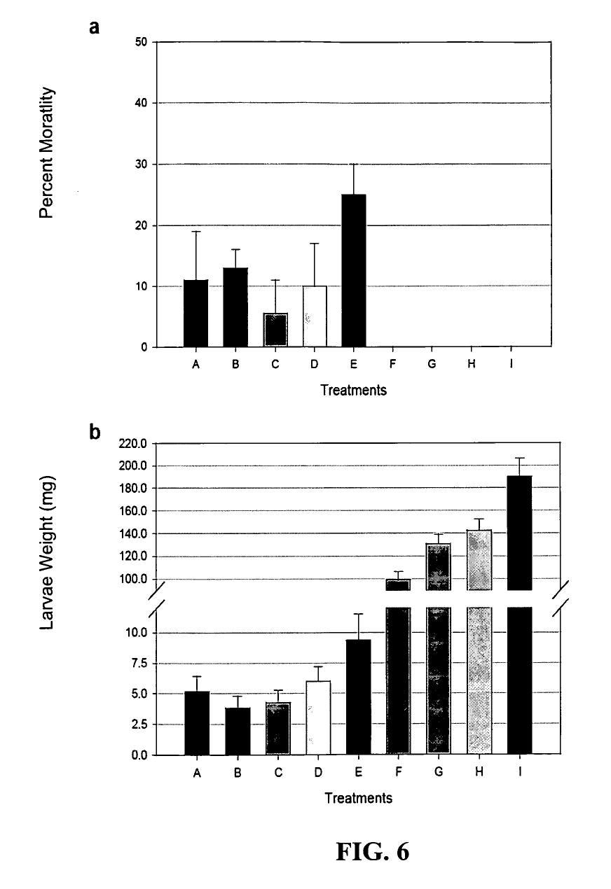

Corn earworm (Helicoverpa zea) larvae are less susceptible to a variety of pesticides including various Bts. A diet overlay bioassay as described above was conducted using flour prepared from plants expressing Cry1Ac and equal quantities of flour prepared from either wildtype plants or from plants expressing CR9-MPED or CR12134. After 7 days mortality was scored and weight of surviving larvae was measured. Co-application of flour from CR12134 plants with flour from Cry1Ac expressing plants enhanced the mortality rate of corn earworm neonates compared to those fed Cry1Ac and wild-type flour as shown in FIG. 6. Co-application of flours prepared from CR12134 and CR9-MPED plants along with Cry1Ac flour also significantly lowered the weight of larvae after 7 days of feeding (FIG. 6b). No insect mortality was observed for insects feeding on diet with flours prepared from wild type, CR9-MPED, or CR12134 plants alone (FIG. 6a). Although there was some variation in survivor weight from these control preparations, their weight was generally an order of magnitude more than survivors feeding on flours with Cry1Ac or any combination of flours with Cry1Ac and BtB proteins (FIG. 6b).

In FIG. 6, sample size: 32 larvae/rep×2 rep/treatment. Bioassay was scored on Day 7. Panel a. Mortality data. Panel b. Surviving larvae weight data (averaged from each group). *Significant enhancement in mortality was observed for Cry1Ac+CR12134 plants (C vs. E). The legend for FIG. 6 is A. 2.0 mg/cm2 Cry1Ac#2; B. 3.0 mg/cm2 Cry1Ac#2; C. 2.0 mg/cm2 Cry1Ac#2+2.0 mg/cm2 wild type Arabidopsis; D. 2.0 mg/cm2 Cry1Ac#2+2.0 mg/cm2 CR9-MPED#1; E. 2.0 mg/cm2 Cry1Ac#2+2.0 mg/cm2 CR12134#6; F. 2.0 mg/cm2 wild type Arabidopsis; G. 2.0 mg/cm2 CR9-MPED#1; H. 2.0 mg/cm2 CR12134#6; and I. Buffer.

### Example 3

### Mosquito-Specific B.t.is and B.t.Bs

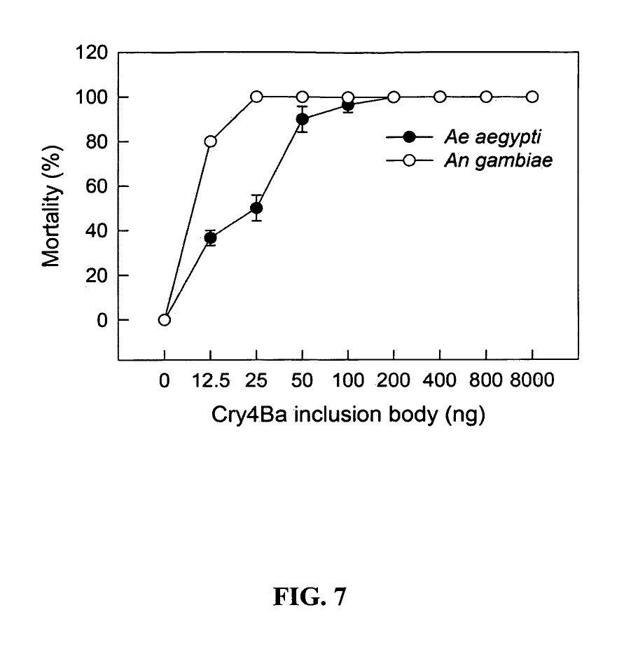

The B.t.i-related toxin Cry4Ba has strong insecticidal activity toward mosquito larvae. Data for two mosquito species Aedes aegypti and Anopheles gambiae are shown in FIG. 7.

Using a directed evolution approach, a novel engineered B.t.i-related toxin Cry4Ba-GAV that would kill an even broader range of mosquito species has been derived from Cry4Ba (Abdullah et al. (2003) Appl. Environ. Microbiol. 69: 5343-5353.) In particular, B.t.i Cry4Ba-GAV has a 700-fold increase of activity against the insect vector Culex quinquefaciatus, 285 fold increase of activity against C. pipiens and a 42,000-fold improvement against Aedes aegypti, relative to the parent Cry4Ba toxin (Abdullah et al. (2003) Appl Environ Microbiol 69: 5343-5353.) Cry4Ba has strong killing activity toward the distantly related black fly species (e.g., Simulium damnosum) that is a prominent vector for the parasites that cause Onchocerciasis (African river blindness) (Dadzie et al. (2003) Filaria J. 2: 2.)

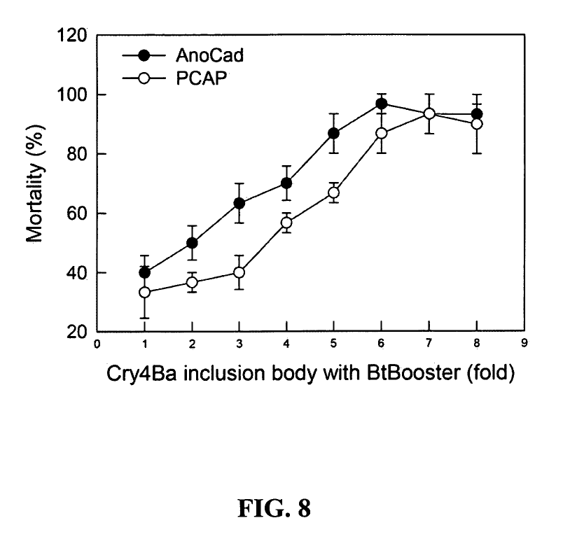

B.t.-booster (B.t.B) proteins enhance the killing activity of B.t.i-related toxins several-fold, making B.t.is more effective at insect larval control. B.t.Bs work by enhancing the uptake of B.t.s in the insect midgut. They have binding specificity for both the particular B.t. being used and membrane proteins in the target insect's midgut (Banks et al. (2001) Insect Biochem. Mol. Biol 31: 909-918); (Hua et al. (2004) J. Biol. Chem. 279: 28051-28056); (Hua et al. (2001) Appl. Environ. Microbiol. 67: 872-879.); (Jurat-Fuentes and Adang (2006b) Biochemistry 45: 9688-96895); (Jurat-Fuentes and Adang (2006a) J Invertebr Pathol 92: 166-171.); (Abdullah et al. (2003) Appl. Environ. Microbiol. 69: 5343-5353.) The data showing that the mosquito-derived B.t.Bs AgCad and PCAP enhanced the killing activity of Cry4BA toward the larvae of the mosquito Aedes aegypti are shown in FIG. 8.

### Example 4

### Methods of Isolating Seed Oil Bodies

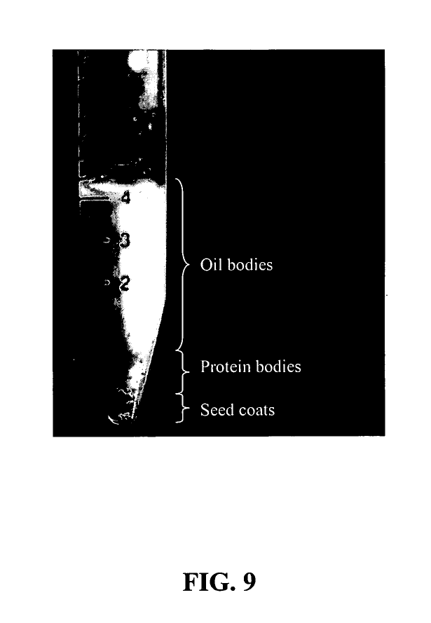

Simple methods release seed oil bodies from the rest of the seed material including homogenizing the seeds in buffer. Simply homogenizing the seeds in 20 parts (w/v) of 10 mM Tris-Buffer (pH=8.0) released 99% of the oil-seed bodies. FIG. 9 shows one such sample in which 200 mg of seeds was homogenized in 4 ml of buffer for 2.5 min. The sample was centrifuged for 10 min at 3000×g to separate the buoyant seed oil bodies from denser protein bodies and seed coats. Samples that were allowed to stand for 48 hours without centrifugation began to separate into the same three fractions, although their boundaries were not as sharply defined.

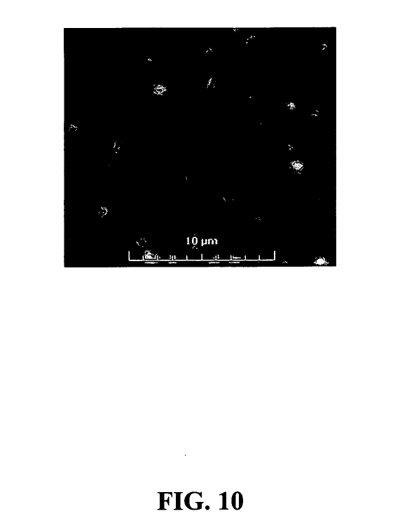

Extensive grinding of the seeds can produce similar preparations of oil bodies, once they are re-suspended in buffer. Various parts of the top layer of the seed oil body fraction in FIG. 10 were examined by microscopy. As shown in FIG. 10, the canola oil bodies from the top fraction varied in size from about 1 to 5 μm, similar to the size of oil bodies from other seed oil plants (Tzen et al. (1997) J. Biochem. (Tokyo) 121: 762-768.) This is similar to the size of most bacterial and small green algal species, which are significant components in mosquito larval diet. The top of the oil body fraction appeared to contain smaller sized bodies than at the bottom, which might affect their sedimentation rates in the water column independent of density. These simple experiments demonstrate that seed oil bodies have diverse densities and sizes that will float at all levels in a water column.

### Example 5

### Generation of an ACTIN7 Controlled Expression Vector (A7pt:: OL1) for the Expression of Protein Tethered to Oleosin in the Membrane Oil Bodies

This example describes novel constructions that will produce recombinant Bti and BtB proteins tethered to oleosins in seed oil bodies that will produce a biologically active mosquitocides.

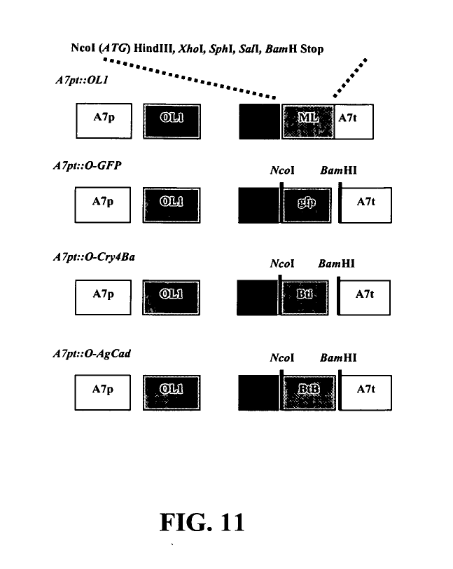

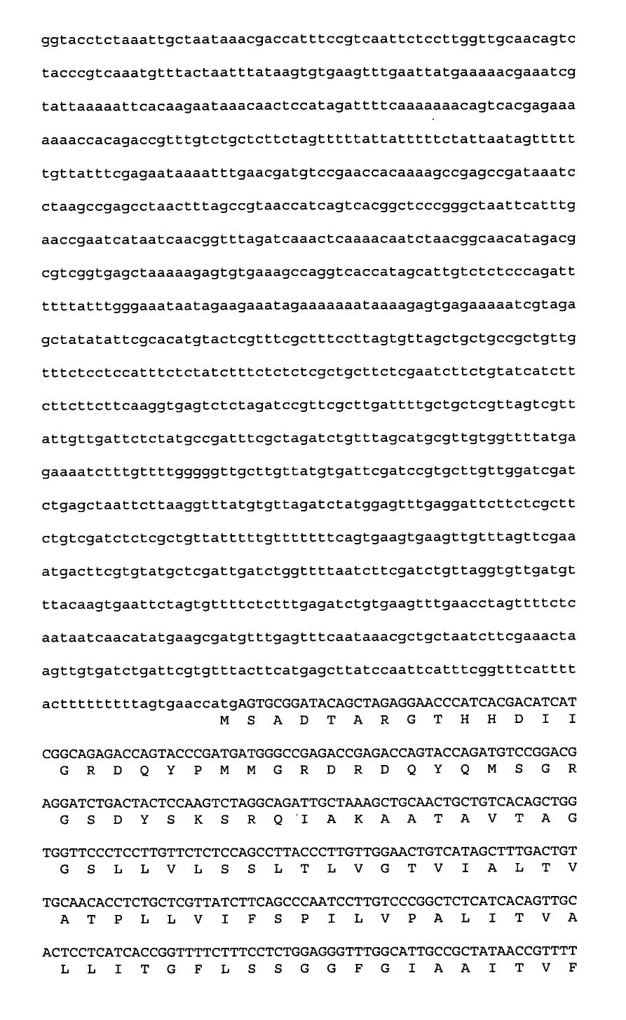

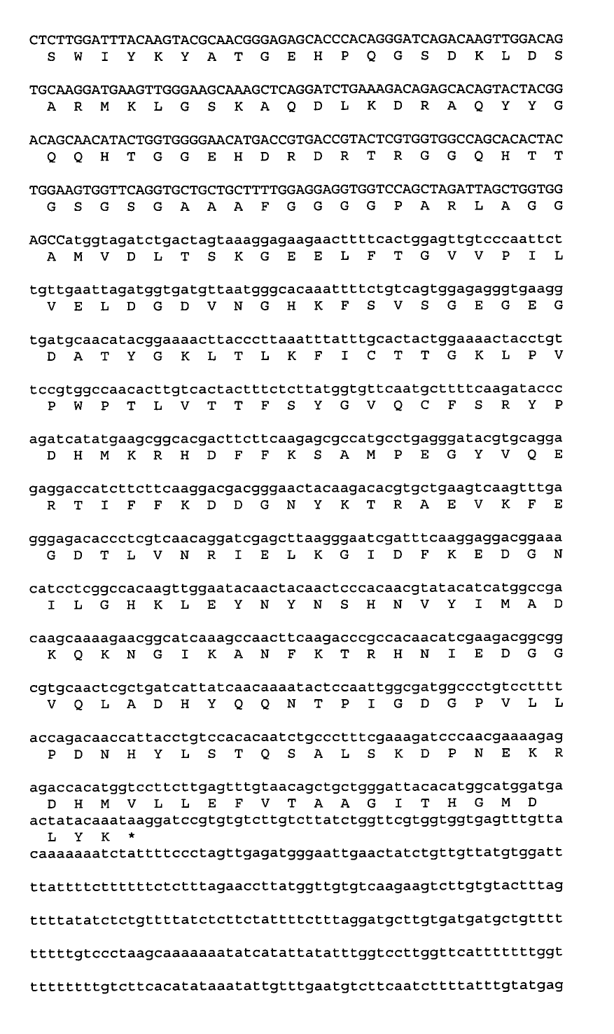

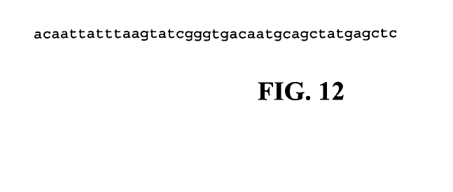

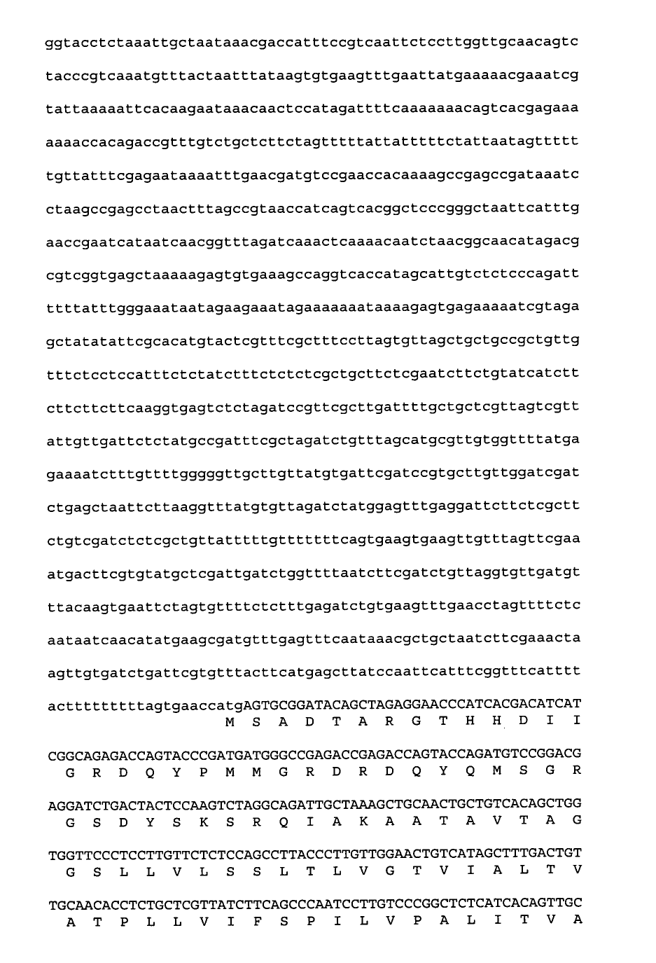

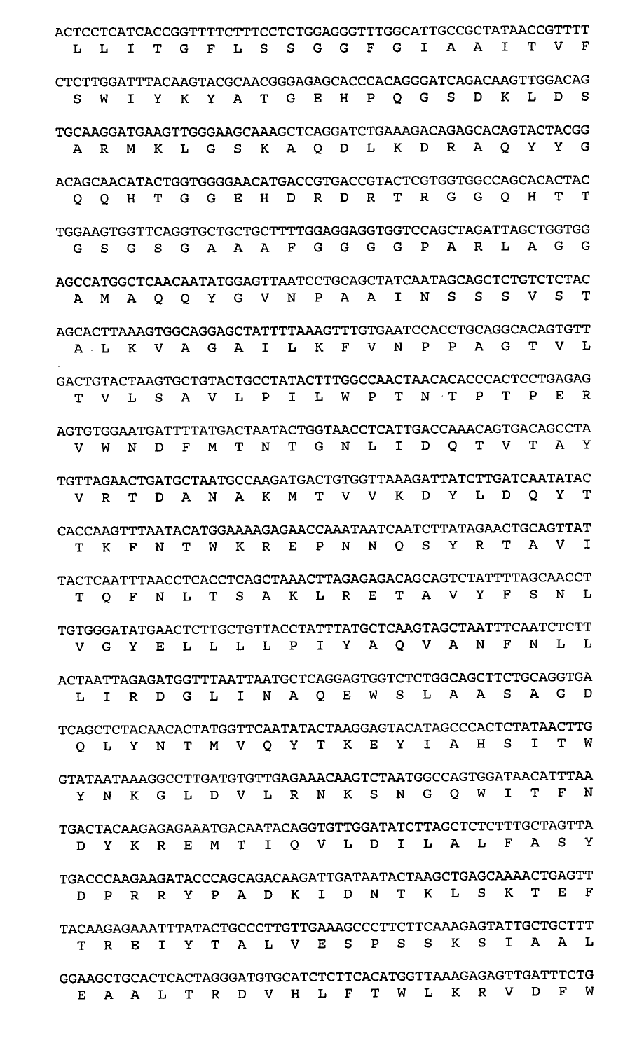

The ACTIN7 gene is strongly expressed in leaves and is nearly as strongly expressed in seeds as oleosin (Zimmermann (2004) Anal Biochem. 78: 47-51.) The previously developed the ACTIN7-based A7pt vector (FIG. 11) maintains the ACTIN7 transcript expression pattern (Kandasamy et al. (2001) Cell 13: 1541-1554.) The A7pt-OL1 vector contains all the necessary information to clone in-translational-frame protein fusions and express their RNAs to the same level as endogenous ACTIN7 mRNA. In addition, the A7pt construct contains the entire OLEOSIN1 protein coding sequence (OL) ending at the terminal amino acid codon (Thr173), but omitting the stop codon (TAA, 174); an in frame pair of protease processing sites (tc), a NcoI (CCATGG) cloning site with an in-frame ATG codon at the start, and a downstream multilinker (ML) region ending in a BamHI cloning site FIG. 11 (SEQ ID NO. 3, FIG. 12.) The redundant protease cleavage site was designed with the sequence AlaAlaAlaPheGlyGlyGly GlyProAlaArgLeuAlaGly, where the peptide bonds following the Phe and Arg residues will be cleaved by trypsin and chymotrypsin, respectively (SEQ ID NO. 3, FIG. 12.)

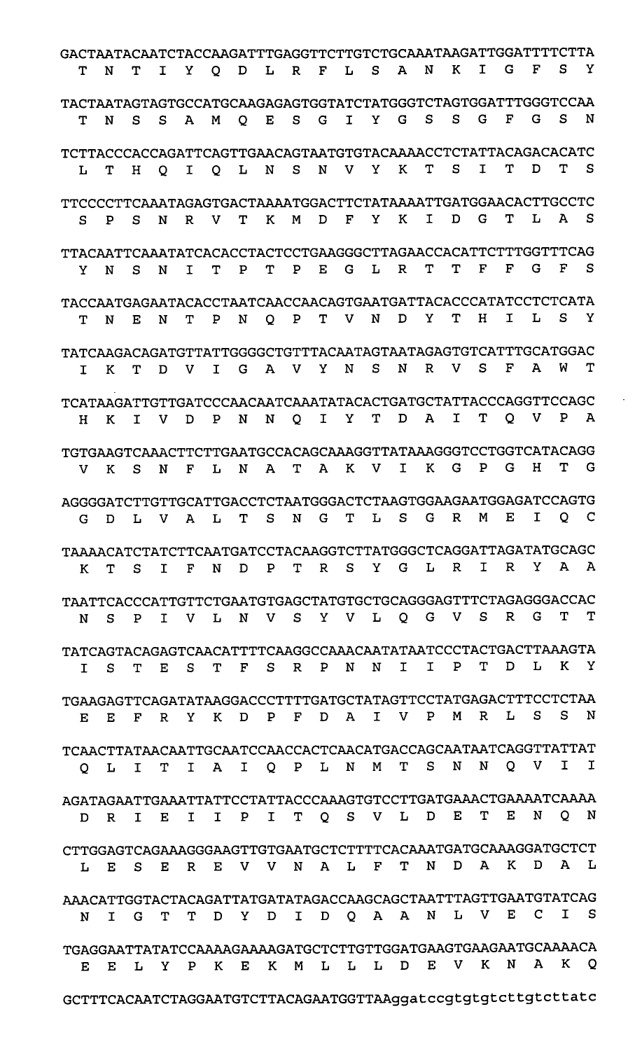

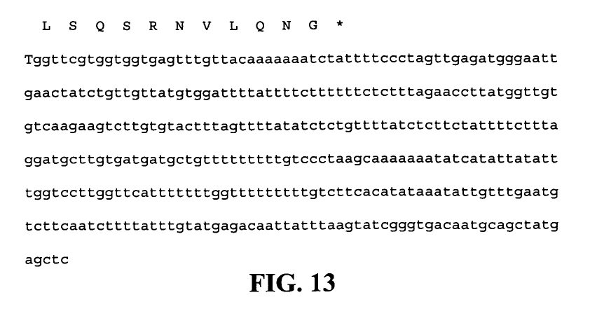

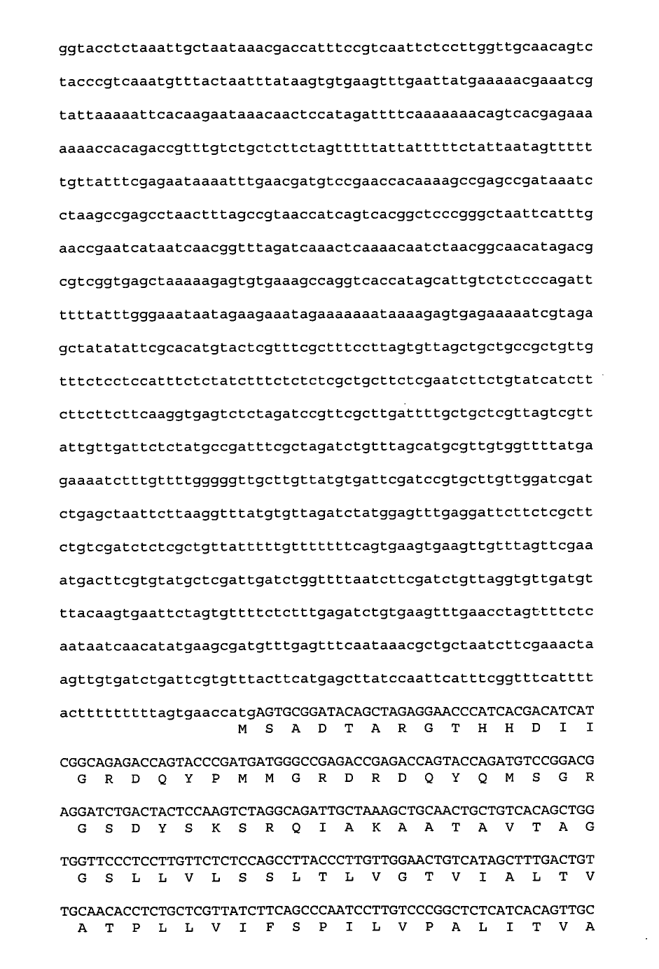

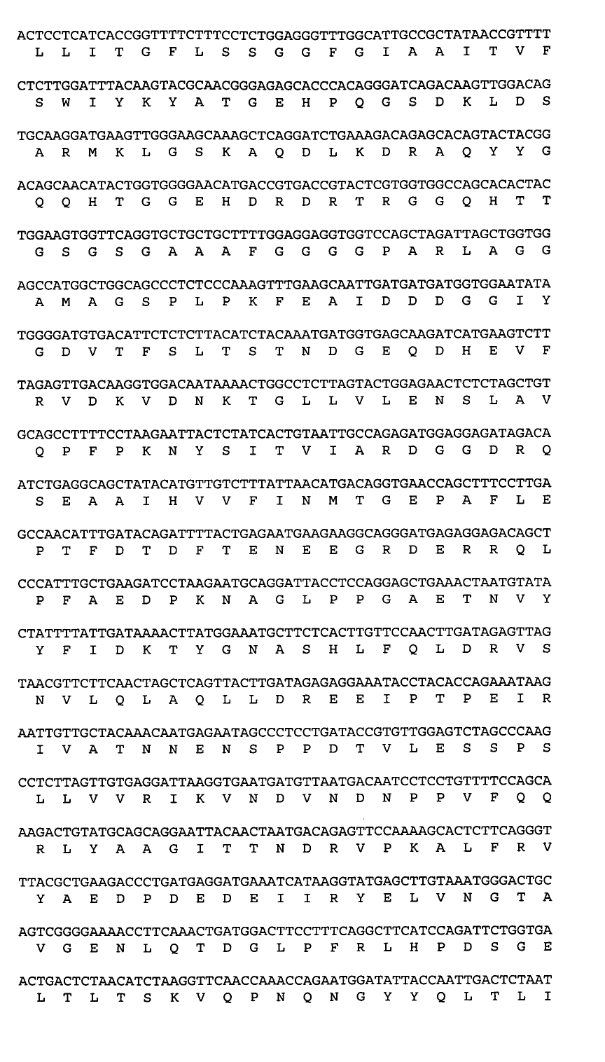

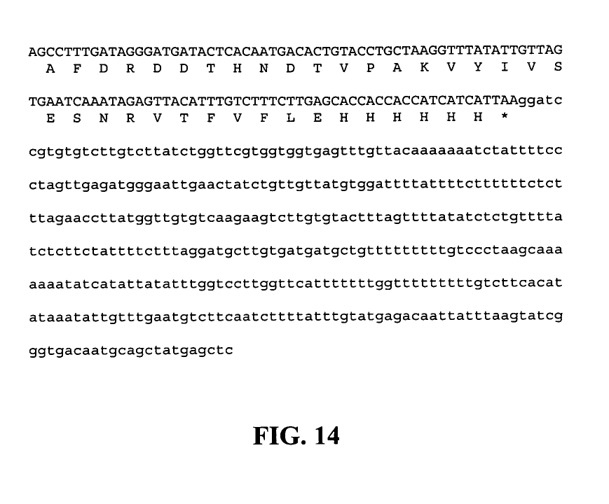

A7pt::OL1 is a vector for expressing oleosin fusion proteins in plants, containing the ACTIN7 promoter (A7p), terminator (A7t), and Oleosin1 cDNA sequences. The tc sequences allow for trypsin and chymotrypsin protease cleavage in vivo. ML is the multilinker for cloning. A7pt::O-GFP, A7pt::O-Bti and A7pt::O-BtB are constructs for expressing of GFP, Bti, and BtB as oleosin fusions in plants, respectively (SEQ ID NO. 3, FIG. 12); (SEQ ID NO. 4, FIG. 13); and (SEQ ID NO. 5, FIG. 14) respectively.

The protease cleavage site tc separating the oleosin from the protein of interest (B.t.i, B.t.B) needs to be efficiently processed in mosquito larval gut. The tc sequence was designed based on consensus sequences for animal tryptic and chymotryptic cleavage sites and well-tested fluorogenic substrates for these two proteases (Zimmerman et al. (1977) Anal Biochem. 78: 47-51); (Graf et al. (1988) Proc. Natl. Acad. Sci. USA 85: 4961-4965); (Kokotos et al. (1990) Biol Chem Hoppe Seyler 371: 835-840); (Weder et al. (1993) Electrophoresis 14: 220-226); (Grahn et al. (1998) Anal. Biochem. 265: 225-31.) The redundant protease cleavage site has the sequence AlaAlaAlaPheGlyGlyGly GlyProAlaArgLeuAlaGly, where the peptide bonds following the Phe and Arg residues will be cleaved by trypsin and chymotrypsin, respectively. The use of two protease- processing sites maximizes cleavage and release of B.t.i and B.t.B from the recombinant oleosin proteins in the insect midgut.

The GFP control reporter construct: The 729 by GFP sequence was PCR amplified from EGAD vector and modified with the PCR primers to contain an in-frame NcoI site at the start codon and BamHI site after the stop codon (Cutler et al. (2000) Proc Natl Acad Sci 97: 3718-3723) This PCR product was cloned into A7pt::OL1 to produce the A7pt::O-GFP reporter control construct in E. coli. A7pt::O-GFP was shuttled into a binary vector and transformed first into Agrobacterium and then into Arabidopsis thaliana (Columbia ecotype) plants. T1 seeds were selected on Hygromycin, seedlings transferred to vertical growth plates. The roots and leaves from ten T1 generation plant lines were examined for GFP fluorescence.

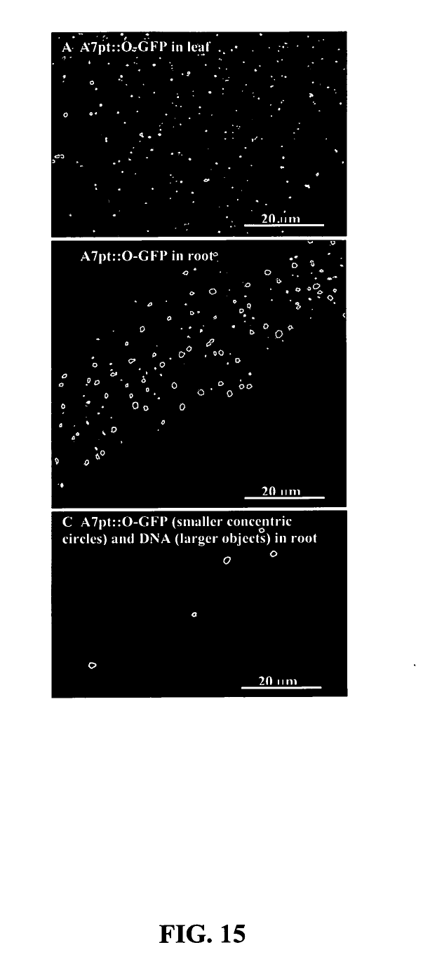

The A7pt::O-GFP construct was designed for several control experiments that will provide necessary information about the behavior of the system for tethering proteins in oil bodies in most organs and tissues. FIG. 15 shows that GFP is tethered to oil bodies in shoots and roots when expressed from an ACTIN7 promoter. These microscopic studies also confirm that there is little GFP released from the oil bodies into the surrounding cytoplasm, for example, by cleavage of the tryptic and chymotryptic sites. It is important that the target protein stays coupled to oleosin until oil bodies enter the gut of mosquitoes. The strong fluorescent GFP signals provide semi-quantitative evidence that the A7pt::O-GFP vector is strongly expressed.

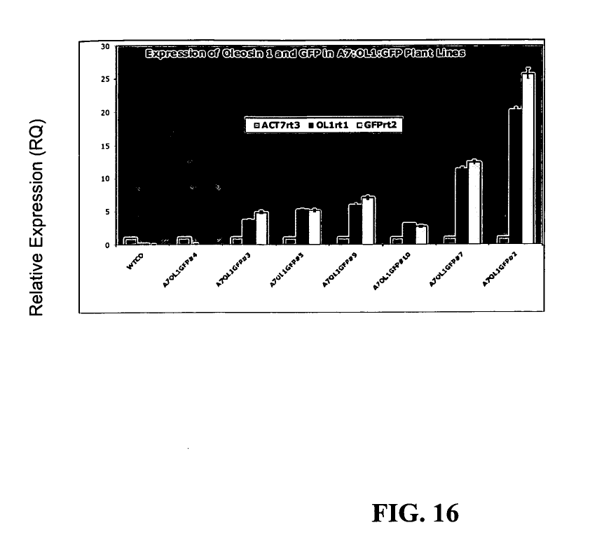

Quantitative data qRT-PCR data demonstrating relatively strong expression of the A7pt::O-GFP transgene in mature leaves are shown in FIG. 16. Endogenous ACTIN7 transcript levels were assayed as normalization control (primer pair ACT7rt3, yellow bars). Two primers were used to assay the levels of the OLEOSIN-GFP fusion mRNA, one primer pair in the OLEOSIN portion of the transcript (OL1rt1) and the one in the GFP portion (GFPrt2). Plant lines scored as having undetected or weak (lines #4 & #3), moderate (#5, #9, #10), and strong GFP fluorescence had approximately proportional levels of the fusion transcript based on primers assaying both parts of the fusion transcript. Furthermore, these data show that the A7pt::OL1 parent vector is capable of driving very high levels of transgene expression relative to endogenous ACTIN7 transcripts.

Bti and BtB coupled to proteins on the surface of seed oil bodies can be delivered effectively as a milled Bt-flour that will float in the water column and kill feeding mosquito larvae.

### Transform Arabidopsis and Regenerate Ten Plants Each Expressing Ol-tc-Cry4Ba and Ol-tc-AgCad

The gene constructs A7pt::OL1-GFP (SEQ ID NO. 3, FIG. 12), A7pt::OL1-Bti (SEQ ID NO. 4, FIG. 13) and A7pt::OL1-AgCad (SEQ ID NO. 5, FIG. 14) were transformed individually into Arabidopsis by Agrobacterium-mediated infiltration of plant inflorescences. The individually transformed lines are selected for HygR this protocol generated heterozygous T1 generation transgenic seeds at a high frequency (˜1% of total seed). T1 seeds of each genotype were collected and germinated under hygromycin selection to produce at least 10 lines expressing each transgene.

Quantitative Real Time (qRT) PCR assays were performed to estimate OL1-GFP, OL1-Bti and OL1-AgCad mRNAs levels relative to endogenous ACTIN7 mRNA levels in developing leaves and seeds. Table 3 and FIG. 16 show the levels of transgenic OL1-GFP mRNA in leaves relative to endogenous ACTIN7 mRNA levels set to equal 1. Separate primers were used for the OLIOSIN and GFP portions of the fused transcript. The primers in these two gene regions showed reasonable agreement in quantifying the levels of GFP transcript expression. In general transgenic leaf transcript levels of OL1-GFP were as high as those for endogenous ACTIN7 or as much as 10 to 20 times higher.

TABLE 3
Summary of OL1-GFP Expression in leaves of
A7:OL1:GFP T1 Plant Lines

| | Ratio of OL1 and GFP to ACT7 |
|---|---|
| Plant Line | ACT7 (1):OL1:GFP |
| WT | 1:0.05:0.0 |
| A7:OL1:GFP #4 | 1:0.02:0.0 |
| A7:OL1:GFP #3 | 1:3.7:4.9 |
| A7:OL1:GFP #5 | 1:5.3:5.2 |
| A7:OL1:GFP #9 | 1:5.9:7.1 |
| A7:OL1:GFP #10 | 1:3.1:2.7 |
| A7:OL1:GFP #7 | 1:11.3:12.4 |
| A7:OL1:GFP #2 | 1:20.2:25.6 |

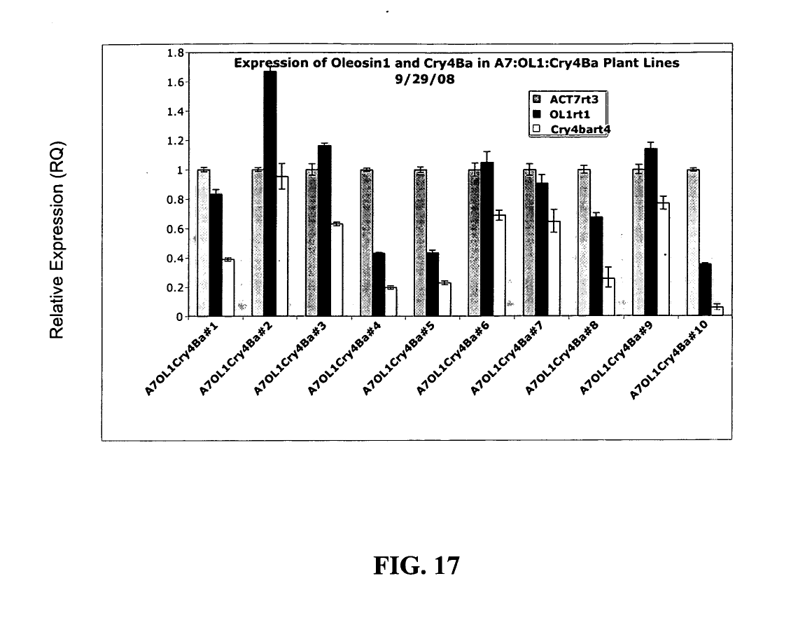

Table 4 and FIG. 17 show the levels of transgenic OL1-CRY4Ba mRNA in leaves relative to endogenous ACTIN7 mRNA levels set to equal 1. Separate primers were used for the OLIOSIN and Cry4BA portions of the fused transcript. The primers in these two gene regions showed reasonable agreement in the levels of transcript expression, although qRT-PCR of the Cry4Ba product appears less efficiently detected or less stable than the OL1 portion. In general transgenic leaf transcript levels averaged a little below those for endogenous ACTIN7.

TABLE 4
OL1-Cry4Ba transcript levels in leaves of A7pt:OL1:Cry4Ba (Bti)
transgenic plants.

| | RQ |
|---|---|
| Plant Line | ACT7:OL1:Cry4Ba |
| A7PT:OL1:Cry4Ba #1 | 1:0.83:0.39 |
| A7PT:OL1:Cry4Ba #2 | 1:1.67:0.95 |
| A7PT:OL1:Cry4Ba #3 | 1:1.16:0.63 |
| A7PT:OL1:Cry4Ba #4 | 1:0.43:0.20 |
| A7PT:OL1:Cry4Ba #5 | 1:0.43:0.23 |
| A7PT:OL1:Cry4Ba #6 | 1:1.05:0.69 |
| A7PT:OL1:Cry4Ba #7 | 1:0.90:0.65 |
| A7PT:OL1:Cry4Ba #8 | 1:0.67:0.26 |
| A7PT:OL1:Cry4Ba #9 | 1:1.14:0.77 |
| A7PT:OL1:Cry4Ba #10 | 1:0.35:0.06 |

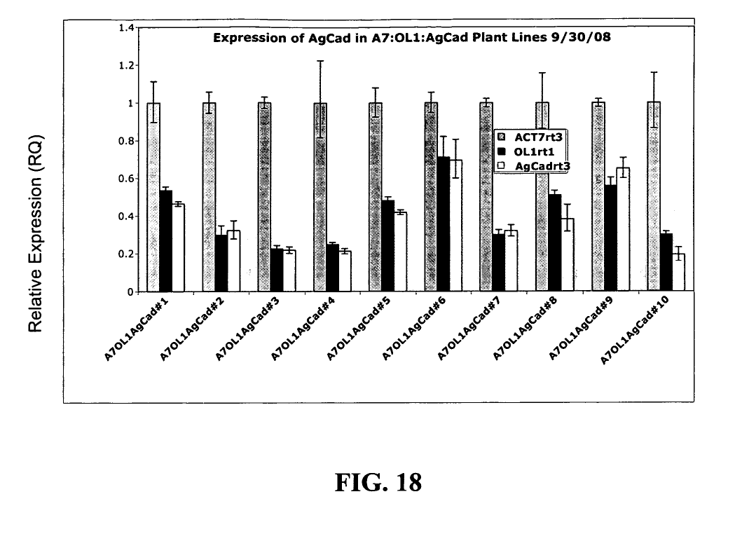

Table 5 and FIG. 18 show the levels of transgenic OL1-AgCad mRNA in leaves relative to endogenous ACTIN7 mRNA levels set to equal 1. Separate primers were used for the OLIOSIN and AgCad portions of the fused transcript. The primers in these two gene regions showed reasonable agreement in the levels of transcript expression. In general transgenic leaf transcript levels averaged a less than half of those for endogenous ACTIN7.

TABLE 5
OL1-AgCAD transcript levels in leaves of A7pt:OL1:AgCad (BtB)
transgenic plants.

| | RQ |
|---|---|
| Plant Line | ACT7:OL1:AgCad |
| A7PT:OL1:AgCad #1 | 1:0.53:0.46 |
| A7PT:OL1:AgCad #2 | 1:0.30:0.32 |
| A7PT:OL1:AgCad #3 | 1:0.22:0.22 |
| A7PT:OL1:AgCad #4 | 1:0.25:0.21 |
| A7PT:OL1:AgCad #5 | 1:0.48:0.42 |
| A7PT:OL1:AgCad #6 | 1:0.71:0.69 |
| A7PT:OL1:AgCad #7 | 1:0.30:0.32 |
| A7PT:OL1:AgCad #8 | 1:0.51:0.38 |
| A7PT:OL1:AgCad #9 | 1:0.56:0.65 |
| A7PT:OL1:AgCad #10 | 1:0.30:0.19 |

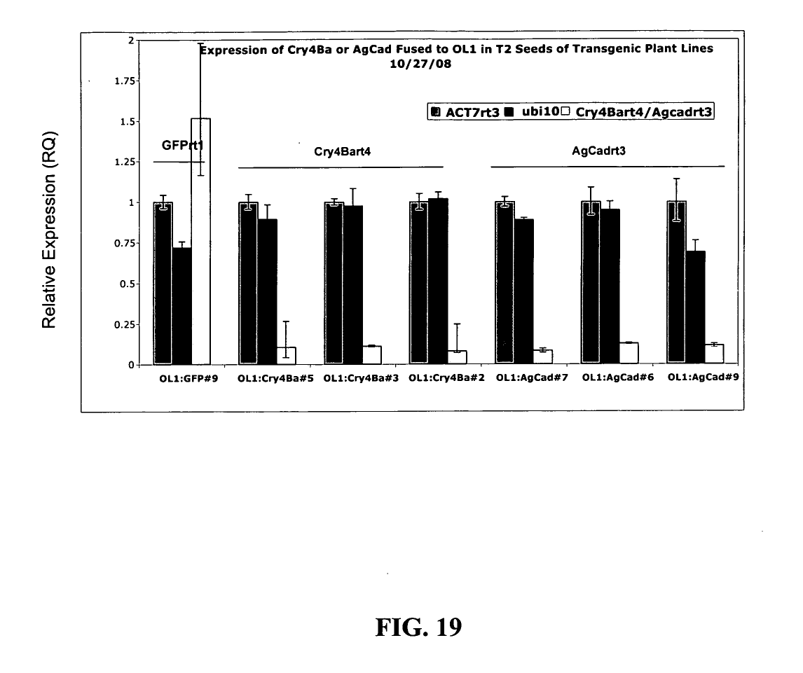

The qRT-PCR assays showed that seed transcript levels were several fold lower than observed in leaves for all three transcripts as summarized in Table 6 and FIG. 19. For example, the levels of transgenic OL1-GFP line #9 were five times lower in seed than in leaf, although the total levels were still above endogenous ACTIN7. However, the levels of OL1 -Cry4Ba and OL1-AgCad transcripts in seeds were even lower. In order to determine if there was some error in the endogenous ACTIN7 control, a second potential normalization control UBIQUITIN10 was included in the assays, but its transcript levels were remarkably similar to those of ACTIN7. In general transgenic seed transcript levels for OL1-Cry4Ba and OL1-AgCad averaged about 10% of those for endogenous ACTIN7. In summary, all three transcripts seem less efficiently expressed in seed than in leaves. The levels of Cry4Ba and AgCad in leaf and seed were very low relative to OL1 -GFP. Thus, the Cry4Ba and AgCad mRNAs may not be very stable. Bioassays for mosquitocidal activity are preferably done with the Cry4Ba plants expressing the highest transcript levels.

TABLE 6
Summary of A7:OL1:Bti or BtBi Expression in
T2 Seeds of Transgenic Plant Lines

| | RQ ratio Act7rt3 to | |
|---|---|---|
| | GFPrt1, Cry4Bart4 or | RQ GFPrt1, Cry4Bart4 or |
| | Agcadrt3 | Agcadrt3 |
| Plant Line | T1 Leaf Tissue | T2 Seeds |
| A7:OL1:GFP #9 | 1.0:7.3 | 1.0:1.5 |
| A7:OL1:Cry4Ba #5 | 1.0:0.23 | 1.0:0.15 |
| A7:OL1:Cry4Ba #3 | 1.0:0.63 | 1.0:0.11 |
| A7:OL1:Cry4ba #2 | 1.0:0.95 | 1.0:0.08 |
| A7:OL1:AgCad #7 | 1.0:0.32 | 1.0:0.08 |
| A7:OL1:AgCAd #6 | 1.0:0.69 | 1.0:0.13 |
| A7:OL1:AgCad #9 | 1.0:0.65 | 1.0:0.11 |

Even though these values of expression in seed are low they are several orders of magnitude above background detected by qRT-PCR in wild type. Wild type background levels of Cry4Ba and AgCad in seed are estimated to RQs less than 0.002 and 0.00025, respectively.

### Assay for the Mosquitocidal Activity of Bt-Flour from A7pt::O-Cry4Ba Plant Lines Expressing Bti Protein Cry4Ba-GAV on Feeding Mosquito Larvae

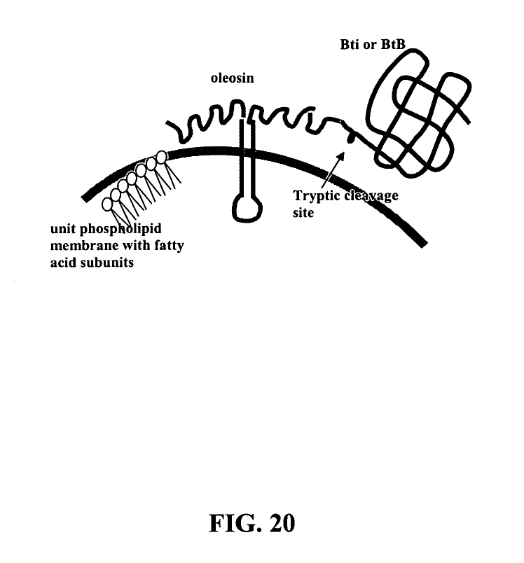

This experiment demonstrates that B.t. tethered to oil bodies (FIG. 20) kills mosquito species that are a particular threat to human health. The ability of the engineered oil bodies to kill three mosquito species: Anopheles gambiae with the Anopheles genera including the only vectors for human malaria; Culex pipens and Culex quinquefasciatis as major vectors for filarisis, West Nile virus, Japanese encephalitis, St. Louis encephalitis, and avian malaria; and Aedes aegypti the vector for yellow fever, equine encephalitis, and dengue were tested. Initial assays have been performed on Culex quinquefasciatis and Aedes aegypti.

Oil bodies (Bt-flour) were prepared by grinding 25 mg of seeds dry in a mortar and pestle under liquid nitrogen and then re-suspending the paste in 500 82 l of 10 mM Tris buffer pH 8.0. Samples were centrifuged at 9,000×g for 1 min to remove seed coats and protein bodies into the pellet, and the supernatant containing the oil body emulsion was removed, stored at 4° C., and assayed within 48 hrs.

Bioassays consisted of ten cups with 1.5 ml of distilled water with varying dilutions of Bt-flour emulsion per plant line (Wild-type plant vs. Cry4Ba-GAV transgenic). For all three mosquito species, ten larvae were used per cup with ten cups per plant line. Bioassays were incubated at 28° C. and repeated twice. Mortality was scored after 24 and 48 hours. Larvae were counted as dead if they do not move upon probing. Error bars represent the Standard Deviation among samples.

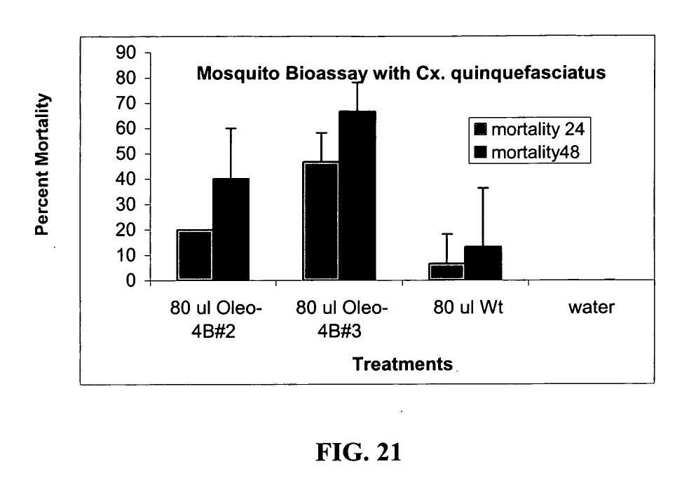

Samples of oil body emulsion were prepared from the seeds of OL1:Cry4Ba lines #2 and lines #3 and wild type (WT) as described above. 80 μl samples of each of these were added to each cup of ten C. quinquefasciatus larvae. The results are shown in FIG. 21. The OL1:Cry4Ba oil bodies killed more effectively than the wild type oil bodies or water controls. The sample of oil bodies from line #3 killed approximately 68% of the larvae in the 48 h. Nearly 60% and 80% killing was observed for oil body samples #2 and #3 after 72 hours (not shown). Although these experiments indicate killing with the WT control oil bodies, previous WT oil body samples generally did not cause any larval mortality. A repeat of this experiment with 20 ul of oil body sample produced nearly as much larval mortality.

Initial experiment examined also killing of Aedes aegypti larvae by OL1:Cry4Ba oil bodies. Samples of oil body emulsion were prepared from the seeds of OL1:Cry4Ba lines #7 and lines #9 and wild type (WT). Approximately 20 μl samples of each of these were added to each ml cup containing five A. aegypti larvae. Only approximately 10% to 30% of the larvae died in any of these experiments after 24, 48 or 72 hours. In these experiments no killing was observed with the control wild type oil bodies. Apparently A. aegypti is less sensitive to the oil body born OL1:Cry4Ba toxin.

### Example 6

### Expression of Formaecin Iₛ (FormIs), a Proline Rich Antibacterial Peptide, in Plants

The expression cassette for PRAPs: The most efficient production of antibacterial PRAP-flours makes use of different aboveground parts of the plant including leaves, stems, and seeds. The approach for expressing PRAPs is technically parallel to that described above, where Bt and BtB proteins were expressed in Arabidopsis. In the PRAP-flours, the ACTIN7 vector system will be used. The ACTIN7 gene and A7pt promoter cassette is as strongly expressed in leaves, stems, and seeds. The A7pt vector contains the necessary ACTIN7 promoter, 5′ UTR, intron and transcriptional enhancers, translational enhancers, inframe multilinker, 3′ UTR, and polyadenylaton sequences to maintain the expression strength and pattern of the native gene.

The double stranded DNA sequences encoding Formacin Is (Table 1) was prepared synthetically and transformed as NcoI/BamHI fragments into the A7pt vector.

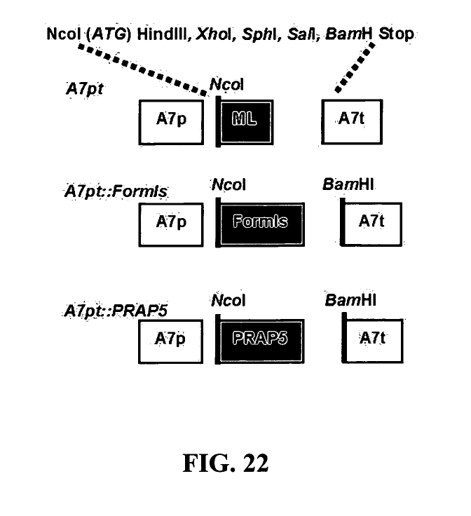

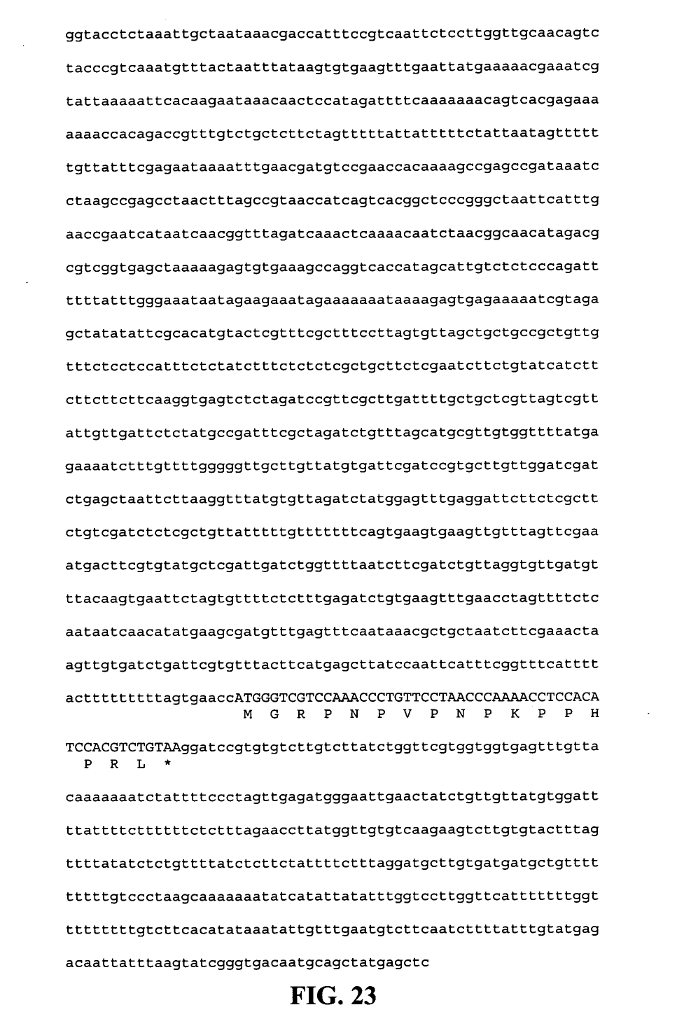

The resulting E. coli clones of A7pt::FormIs and A7pt::PRAP5 (FIG. 22) were analyzed to confirm its DNA sequence (SEQ ID NO. 6, FIG. 23). The pCambia-derived binary plasmid was purified and transformed into an Agrobacterium tumefaciens strain that contained all the genes necessary to transform these sequences into plants on a second plasmid. The vector contained the HygR (plant hygromycin resistance gene) such that the transgene was linked to a plant expressed hygromycin resistance gene HygR during plant transfer.

Plant transformation: The gene constructs A7pt::FormIs (FIG. 22) were transformed individually into Arabidopsis by Agrobacterium-mediated infiltration of plant inflorescences (Ye et al. (1999) The Plant Journal 19: 249-257.) When the individually transformed lines were selected for HygR this protocol generated heterozygous T1 seeds (first generation hemizygous transgenic seeds) at a high frequency (˜1% of total seed). Because about 25,000 seeds were produced per transformation, T1 transformants were produced in excess. T1 seeds of each genotype were collected and germinated under hygromycin selection to produce 10 lines expressing each transgene. Ten desired transgenic Arabidopsis lines were produced within 10 weeks of starting.

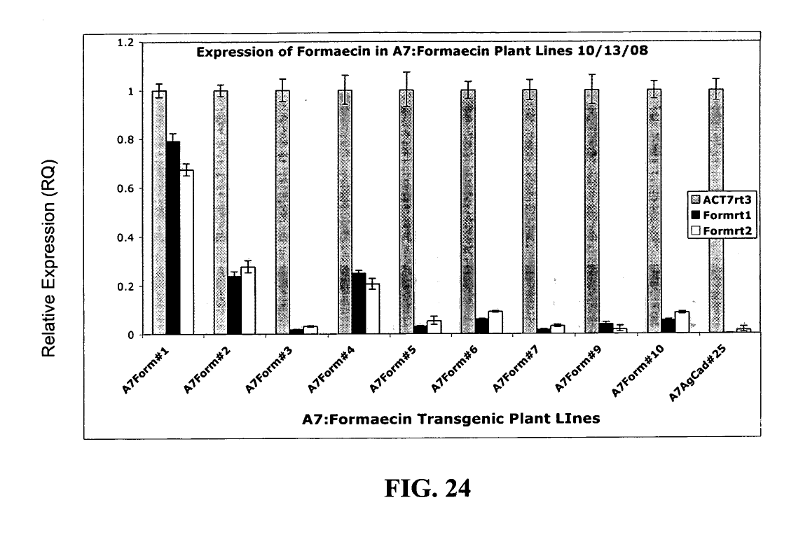

Quantitative Real Time (qRT) PCR assays were performed to estimate Formacin Is mRNAs levels relative to endogenous ACTIN7 mRNA levels in leaves and seeds of each plant line. This can be parallel to the use of ACTIN2 mRNA as a control in leaves when using the A2pt vector system. A set of plants with varying levels of expression were obtained, however the expression levels were not very high generally being less than 20% of that for the ACTIN7 transcript control. However, one plant line #1 produced levels of FormIₛ RNA that were nearly equal to that of shown in FIG. 24. Plants with the highest levels of Formaecin Is expression relative to ACTIN7 will be propagated further and assayed for toxicity to bacterial target organisms.

Preparation of antibacterial-flours: Antibacterial-flours will be prepared from the lyophilized leaves and seeds of the the lines expressing the highest levels of A7pt::FormIs and from wild-type (WT) control plants. A standard grinding technique in which liquid nitrogen is mixed with 1 g of the dried plant material and the mixture ground in a mortar and pestle can be used. The powder will be weighed into 100 mg aliquots of BRAP-flour and stored at −70° C. Some samples will by lyophilized and ground and stored as a dried powder. An aqueous extract will be made from other samples as follows: 100 mg tissue samples will be extracted with 2 volumes (wt/vol) of ice-cold PBT medium (20 mM sodium phosphate buffer, pH 7.0, 0.02% Tween 20, 0.03%).

After several minutes of vortex mixing, the extract is centrifuged at 10,000×g for 10 min in the cold and the supernatants saved. These ˜200 μl aliquots of PRAP-flour and WT-flour extracts will be stored frozen at −70° C.

Demonstration that extracts from PRAP-expressing plants kills S. enterica serotype Enteritidis and E. coli K12 species relative to extracts from wild-type plants. Radial diffusion assays giving a zone of inhibition (ZOI) and minimum inhibitor concentration (MIC) assays will be performed on two coliform indicator species. Salmonella Enteritidis PT13a strain 21027 (bf) forms biofilms, but contains mutations making it a safer laboratory strain. It has a partially sequenced genome and numerous available SNPs (known single nucleotide polymorphisms). It is widely used in research involving Salmonella in the food industry and related strains are routinely isolated from chicken products (Morales et al. (2006) FEMS Microbiol Lett. 264: 48-58); (Guard-Bouldin et al. (2007) Appl. Environ. Microbiol. 73: 7753-7756); and (Morales et al. (2007) Environ. Microbiol. 9: 1047-1059.)

Escherichia coli K12 MG1655 in as close to wild type E. coli as can be found and contains the first sequenced bacterial genome. It is a widely used as the control in tests of antibiotic sensitivity and resistance. By testing both S. Enteritidis and E. coli species, it is possible to obtain a better initial indication of the antibacterial activity of the PRAPs under study.

Radial diffusion assays: The following radial diffusion assays are taken from those previously described for analyses of antibacterial peptides (Nagpal et al. (1999) J. Biol. Chem. 274: 23296-232304); and (Kaur et al. (2007) Protein Sci. 16: 3009-315), the radial diffusion assays of both of which are herein incorporated by reference in their entirety) and for analyses of toxic heavy metals and metalloids as described by (Rugh et al. (1996) Proc. Nat'l Acad. Sci. U.S.A. 93: 3182-3187); (Bizily et al. (1999) Plant Physiol. 131: 463-471); (Dhankher et al. (2002) Nat. Biotechnol. 20: 1140-1145); and (Dhankher et al. (2003) New Phytologist 159: 431-441), which disclosed assays are also herein incorporated by reference in their entirety. Bacteria are grown overnight at 37° C. in 10 ml of full strength (3% w/v) Tryptic Soy Broth (TSB). In the morning 1 ml of this culture is inoculated into 10 ml of fresh TSB and incubated for an additional 3 h at 37° C. to obtain early to mid-logarithmic phase organisms. About 1 to 3×10⁶ cells are then mixed with 1% agarose in PBTT medium (20 mM sodium phosphate buffer, pH 7.4, 0.02% Tween 20, 0.03% in TSB). The mixture is poured onto the agar surface of standard round Petri plates (3% TSB, 1% Agarose) and rapidly dispersed. A 5 mm diameter well is then made in the plate using suction to remove the agar plug. A 10 μl aliquot of liquid extract from each PRAP-flour extract and WT-flour extract (negative control) is placed in each well made in the agarose and then incubated at 37° C. overnight. Ten- and 100-fold dilutions of the plant samples will also be assayed. The diameter of the clear zone surrounding the well (ZOI) is measured for the quantification of inhibitory activities.

Synthetic peptide positive controls: Formaecin Is (Table 7) will be synthesized commercially (Peptide 2.0, Chantilly, Va.) and made up into a 1 mM stock in PBST (˜2 mg/ml for both peptides). A 10 ul aliquot (10 nmole) of this stock and additional freshly made 10-fold dilutions in PBT will be assayed in parallel with the PRAP-flour extracts in ZOI assays. Previous reports suggest that as little as 10 pmoles of active peptide can give a significant ZOI above background. These assays will not only act as positive controls linking our work to the published literature on the PRAPs, but they will allow for an estimation of the concentration of peptide released from the plant-derived PRAP-flours.

MIC Assays: Antibacterial growth inhibition assays modified from assays previously described for antibacterial peptides (Cudic et al. (2002) Peptides 23: 2071-2083) and (Otvos et al. (2005) J Med Chem 48: 5349-5359) will be performed in liquid culture using sterile 96-well microtiter plates in a final volume of 100 μl. The cell concentrations in colony-forming units (cfu) is estimated from ultraviolet absorbance (A) at 600 nm, where A600=1 is 3.8×108 cfu/ml. Mid-logarithmic phase bacterial cultures grown in full-strength Muller-Hinton broth are diluted to A600=0.001 (4×10⁵ cfu/ml). Ninety μl of these cells are added to 10 μl of serially diluted peptides dissolved in PBT. Cultures are then incubated at 37° C. for 16-20 h without shaking, and growth inhibition is measured by recording the absorbance at A600 nm using a microplate reader. MICs are identified as the lowest antimicrobial doses when the A600 nm absorbance did not exceed the negative control medium only values. The ID50 (inhibitory dose giving 50% growth) data are calculated by averaging the absorbance figures of no growth and full bacterial growth and estimating the antibiotic concentration that gives this level of inhibition.

Positive controls will be run with commercially synthesized Formaecin Is peptide samples. Starting with 10 μl of a 10-fold dilution of the 1 mM peptide stock, the highest final peptide concentration will hence be ˜20 μg/ml (1 nmole). Again these controls will allow for a comparison of the MIC data to those in the literature and to estimate peptide concentrations in plant extracts.

Statistical Analysis: Pair-wise Chi-square analysis will be used to analyze the differences in LD50 and MIC estimated from repetitions of these experiments. The difference is considered significant if P<0.05.

Antibacterial-flours from PRAP plants significantly suppress Salmonella and E. coli populations, when fed to litter beetles. When litter beetles eat their normal diet dusted with antibacterial flours prepared from formaecin Iₛ-expressing plants, the number of S. Enteritidis and E. coli they carry will be significantly reduced.

Litter beetle larvae, collected from local poultry houses, will be grown on a diet of chicken feed (Purina Mills, Start and Grow) with and without contaminating indicator bacteria (107 S. Enteritidis or E. coli cells/gram of feed). After 48 h on each diet they will be transferred to a bacteria-free diet for 24 h.

Next, the larvae will be fed a diet of chicken feed dusted with 0, 1, 2.5, and 5% (wt flour/wt feed) of antibacterial-flour and WT-flour. After 48 hours insects will be harvested and DNA extracted (Invitrogen).

S. Enteritidis and E. coli populations will be estimated in several individual insects harvested for each treatment in multiple experiments. Fluorescence-based quantitative real-time PCR (FQ-PCR) will be used to examine the frequency of three independent DNA markers specific for each bacterial strain.

Markers for specific strains of S. Enteritidis are well described at a NCBI web site for Salmonella SNPs (www.ncbi.nlm.nih.gov/genomes/static/Salmonella_SNPS.html). FQ-PCR primers that have been described for the adenylate cyclase gene cyaA and two 23S ribosomal protein subunit genes rr1A and rr1C can be used as markers. These genes have been used as markers to monitor S. Enteritidis in the poultry industry (Morales et al. (2007) Environ. Microbiol. 9: 1047-1059.) SNPs have been widely used to examine the diversity of E. coli species (Hommais et al. (2005) Appl. Environ. Microbiol. 71: 4784-4792) and (Zhang et al. (2006) Genome Res. 16: 757-767.) More general FQ-PCR primers can be designed to assay the homologous cyaA, rr1A, and rr1C genes in WT E. coli MG1655. The genome sequence of strain MG1655 is described at NCBI (Accession #NC_{—}000913).

Oleosin vectors and fusions: The structure of the oleosin protein coding region of the oleosin fusion vector is based on that of the OLEOSIN1 gene (AT4G25140.1) as described in (Rooijen and Moloney (1995) Biotechnology 13: 72-77) however all flanking sequences on both the N- and C-terminal ends are changed. If necessary to obtain the correct distribution of cloning sites one can combine OLEOSIN1 sequences with sequences from OLEOSIN2, 3, 4, and OLEOSIN5 (AT5G40420, AT5G51210, AT3G27660, AT3G01570, respectively).

Each of the following references is herein incorporated by reference in their entirety.
Abdullah, M. A., Alzate, O., Mohammad, M., McNall, R. J., Adang, M. J., and Dean, D. H. (2003). Introduction of Culex toxicity into Bacillus thuringiensis Cry4Ba by protein engineering. Appl Environ Microbiol 69, 5343-53.
Abdullah, M. A., and Dean, D. H. (2004). Enhancement of Cry19Aa mosquitocidal activity against Aedes aegypti by mutations in the putative loop regions of domain II. Appl Environ Microbiol 70, 3769-71.
Abdullah, M. A., Valaitis, A. P., and Dean, D. H. (2006). Identification of a Bacillus thuringiensis Cry11Ba toxin-binding aminopeptidase from the mosquito, Anopheles quadrimaculatus. BMC Biochem. 7, 16.
Aly, C., Mulla, M. S., Schnetter, W., and Xu, B. Z. (1987). Floating bait formulations increase effectiveness of Bacillus thuringiensis var. israelensis against Anopheles larvae. J Am Mosq Control Assoc 3, 583-8.
An, Y. Q., McDowell, J. M., Huang, S., McKinney, E. C., Chambliss, S., and Meagher, R. B. (1996). Strong, constitutive expression of the Arabidopsis ACT2/ACT8 actin subclass in vegetative tissues. Plant J 10, 107-21.
Banks, D. J., Jurat-Fuentes, J. L., Dean, D. H., and Adang, M. J. (2001). Bacillus thuringiensis Cry1Ac and Cry1Fa delta-endotoxin binding to a novel 110 kDa aminopeptidase in Heliothis virescens is not N-acetylgalactosamine mediated. Insect Biochem. Mol Biol 31, 909-18.
Becker, G. D., Adams, L. A., and Hackett, J. (1994). Collagen-assisted healing of facial wounds after Mohs surgery. Laryngoscope 104, 1267-70.
Betz, F. S., Hammond, B. G., and Fuchs, R. L. (2000). Safety and advantages of Bacillus thuringiensis-protected plants to control insect pests. Regulatory Toxicology and Pharmacology 32, 156-173.
Bizily, S. P., Kim, T., Kandasamy, M. K., and Meagher, R. B. (2003). Subcellular targeting of methylmercury lyase enhances its specific activity for organic mercury detoxification in plants. Plant Physiol 131, 463-71.
Calibeo-Hayes, D., Denning, S. S., Stringham, S. M., and Watson, D. W. (2005). Lesser mealworm (Coleoptera: Tenebrionidae) emergence after mechanical incorporation of poultry litter into field soils. J Econ Entomol 98, 229-35.
Capuano, F., Beaudoin, F., Napier, J. A., and Shewry, P. R. (2007). Properties and exploitation of oleosins. Biotechnol. Adv. 25, 203-6.
Chapman, H. C. (1974). Biological control of mosquito larvae. Annu Rev Entomol 19, 33-59.
Carraminana, J. J., Yanguela, J., Blanco, D., Rota, C., Agustin, A. I., and Herrera, A. (1997). Potential virulence determinants of Salmonella serovars from poultry and human sources in Spain. Vet Microbiol 54, 375-83.
Che, D. S., Meagher, R. B., Kim, R., Heaton, A. C. P., Lima, A., Rugh, C. L., and Merkle, S. A. (2006). Expression of methylmercury lyase in Eastern cottonwood (Populus deltoides) confers organic mercury reduction and resistance. In Vitro Cellular. and Developmental Biology-Plant In Press,
Chen, J., Hua, G., Jurat-Fuentes, J, Abdullah, M., and Adang, M. (2007) Synergism of Bacillus thuringiensis toxins by a fragment of toxin-bindig cadherin. PNAS 104, 35, 13901-13906.
Chen, J., Brown, M. R., Hua, G., and Adang, M. J. (2005). Comparison of the localization of Bacillus thuringiensis Cry1A delta-endotoxins and their binding proteins in larval midgut of tobacco hornworm, Manduca sexta. Cell Tissue Res 321, 123-9.
Cudic, M., Condie, B. A., Weiner, D. J., Lysenko, E. S., Xiang, Z. Q., Insug, O., Bulet, P., and Otvos, L., Jr. (2002). Development of novel antibacterial peptides that kill resistant isolates. Peptides 23, 2071-83.
Dadzie, Y., Neira, M., and Hopkins, D. (2003). Final report of the Conference on the eradicability of Onchocerciasis. Filaria J 2, 2.
Dhankher, O. P., Rosen, B. P., Fuhrman, M., and Meagher, R. B. (2003). Increased cadmium tolerance and accumulation by plants expressing bacterial arsenate reductase. New Phytologist 159, 431-441.
Dhankher, O. P., Li, Y., Rosen, B. P., Shi, J., Salt, D., Senecoff, J. F., Sashti, N. A., and Meagher, R. B. (2002). Engineering tolerance and hyperaccumulation of arsenic in plants by combining arsenate reductase and gamma-glutamylcysteine synthetase expression. Nat Biotechnol. 20, 1140-5.
Debbia, E. A. (2000). [Peptide antibiotics]. Recenti Prog Med 91, 106-8.
Delatte, S. J., Evans, J., Hebra, A., Adamson, W., Othersen, H. B., and Tagge, E. P. (2001). Effectiveness of beta-glucan collagen for treatment of partial-thickness burns in children. J Pediatr Surg 36, 113-8.
Delourme, R., Falentin, C., Huteau, V., Clouet, V., Horvais, R., Gandon, B., Specel, S., Hanneton, L., Dheu, J. E., Deschamps, M., Margale, E., Vincourt, P., and Renard, M. (2006). Genetic control of oil content in oilseed rape (Brassica napus L.). Theor Appl Genet 113, 1331-45.
Dhankher, 0.P., Rosen, B. P., McKinney, E. C., and Meagher, R. B. (2006). Hyperaccumulation of arsenic in the shoots of Arabidopsis silenced for arsenate reductase (ACR2). Proc Natl Acad Sci USA 103, 5413-5418.
Francis, B. R., and Bulla, L. A., Jr. (1997). Further characterization of BT-R1, the cadherin-like receptor for Cry1Ab toxin in tobacco hornworm (Manduca sexta) midguts. Insect Biochem. Mol. Biol 27, 541-50.
Graf, L., Jancso, A., Szilagyi, L., Hegyi, G., Pinter, K., Naray-Szabo, G., Hepp, J., Medzihradszky, K., and Rutter, W. J. (1988). Electrostatic complementarity within the substrate-binding pocket of trypsin. Proc Natl Acad Sci USA 85, 4961-5.
Grahn, S., Kurth, T., Ullmann, D., and Jakubke, H. D. (1999). S′ subsite mapping of serine proteases based on fluorescence resonance energy transfer. Biochim Biophys Acta 1431, 329-37.
Grahn, S., Ullmann, D., and Jakubke, H. (1998). Design and synthesis of fluorogenic trypsin peptide substrates based on resonance energy transfer. Anal Biochem. 265, 225-31.
Hancock, R. E., and Chapple, D. S. (1999). Peptide antibiotics. Antimicrob Agents Chemother 43, 1317-23.
Hobbs, D. H., Flintham, J. E., and Hills, M. J. (2004). Genetic control of storage oil synthesis in seeds of Arabidopsis. Plant Physiol 136, 3341-9.
Hommais, F., Pereira, S., Acquaviva, C., Escobar-Paramo, P., and Denamur, E. (2005). Singlenucleotide polymorphism phylotyping of Escherichia coli. Appl Environ Microbiol 71, 4784-92.
Hua, G., Jurat-Fuentes, J. L., and Adang, M.J. (2004). B.t.-R1a extracellular cadherin repeat 12 mediates Bacillus thuringiensis Cry1Ab binding and cytotoxicity. J Biol Chem 279, 28051-6.
Hua, G., Masson, L., Jurat-Fuentes, J. L., Schwab, G., and Adang, M. J. (2001). Binding analyses of Bacillus thuringiensis Cry delta-endotoxins using brush border membrane vesicles of Ostrinia nubilalis. Appl Environ Microbiol 67, 872-9.
Hua, G., Zhang, Z., Abdullah, M. A. F., and Adang, M. J. (2008). Anopheles gambiae cadherin AgCad1binds Cry4Ba toxin of Bacillus thuringiensis israelensis and a fragment of AgCad1 synergizes toxicity.Biochem. 47, 5101-10.
Huang, S., An, Y. Q., McDowell, J. M., McKinney, E. C., and Meagher, R. B. (1997). The Arabidopsis ACT11 actin gene is strongly expressed in tissues of the emerging inflorescence, pollen, and developing ovules. Plant Mol Biol 33, 125-39.
Jako, C., Kumar, A., Wei, Y., Zou, J., Barton, D. L., Giblin, E. M., Covello, P. S., and Taylor, D.C. (2001). Seed-specific over-expression of an Arabidopsis cDNA encoding a diacylglycerol acyltransferase enhances seed oil content and seed weight. Plant Physiol 126, 861-74.
Jako, C., Kumar, A., Wei, Y., Zou, J., Barton, D. L., Giblin, E. M., Covello, P. S., and Taylor, D. C. (2001). Seed-specific over-expression of an Arabidopsis cDNA encoding a diacylglycerol acyltransferase enhances seed oil content and seed weight. Plant Physiol 126, 861-74.
Jolivet, P., Roux, E., D'Andrea, S., Davanture, M., Negroni, L., Zivy, M., and Chardot, T. (2004). Protein composition of oil bodies in Arabidopsis thaliana ecotype WS. Plant Physiol Biochem. 42, 501-9.
Jurat-Fuentes, J. L., and Adang, M. J. (2006a). Cry toxin mode of action in susceptible and resistant Heliothis virescens larvae. J Invertebr Pathol 92, 166-71.
Jurat-Fuentes, J. L., and Adang, M. J. (2006b). The Heliothis virescens cadherin protein expressed in Drosophila S2 cells functions as a receptor for Bacillus thuringiensis Cry1A but not Cry1Fa toxins. Biochemistry 45, 9688-95.
Kandasamy, M. K., Burgos-Rivera, B., McKinney, E. C., Ruzicka, D., and Meagher, R. B. (2007 (In Press)). Class-specific interaction of profilin and ADF isovariants with actin in the regulation of plant development. Plant Cell 5, 263-275.
Kandasamy, M. K., Deal, R. B., McKinney, E. C., and Meagher, R. B. (2005a). Silencing the nuclear actin-related protein AtARP4 in Arabidopsis has multiple effects on plant development, including early flowering and delayed floral senescence. Plant J 41, 845-58.
Kandasamy, M. K., McKinney, E. C., Deal, R. B., and Meagher, R. B. (2005b). ARP7 is an essential actin-related protein required for embryo development and its knockdown affects plant architecture and flower senescence in Arabidopsis. Plant Physiol 138, 2019-2032.
Kandasamy, M. K., Thorsness, M. K., Rundle, S. J., Goldberg, M. L., Nasrallah, J. B., and Nasrallah, M. E. (1993). Ablation of Papillar Cell Function in Brassica Flowers Results in the Loss of Stigma Receptivity to Pollination. Plant Cell 5, 263-275.
Kaur, K. J., Pandey, S., and Salunke, D. M. (2007). Design of a functionally equivalent nonglycosylatedanalog of the glycopeptide antibiotic formaecin I. Protein Sci 16, 309-15.
Killeen, G. F. (2003). Following in Soper's footsteps: northeast Brazil 63 years after eradication of Anopheles gambiae. Lancet Infect Dis 3, 663-6.
Killeen, G. F., Fillinger, U., Kiche, I., Gouagna, L. C., and Knols, B. G. (2002). Eradication of Anopheles gambiae from Brazil: lessons for malaria control in Africa? Lancet Infect Dis 2, 618-27.
Kim, T., Balish, R. S., Heaton, A. C. P., McKinney, E. C., and Meagher, R. B. (2005). Engineering a Root-Specific, Repressor-Operator Gene Complex. Plant Biotech 3, 571-582.
Kokotos, G., Geiger, R., and Tzougraki, C. (1990). Fluorogenic substrates for chymotrypsin with 2-quinolinone derivatives as leaving groups. Biol Chem Hoppe Seyler 371, 835-40.
Lacey, L. A., and Inman, A. (1985). Efficacy of granular formulations of Bacillus thuringiensis (H-14) for the control of Anopheles larvae in rice fields. J Am Mosq Control Assoc 1, 38-42.
Li, Y., Beisson, F., Pollard, M., and Ohlrogge, J. (2006a). Oil content of Arabidopsis seeds: the influence of seed anatomy, light and plant-to-plant variation. Phytochemistry 67, 904-15.
Li, Y., Dankher, O. P., Carreira, L., Smith, A. P., and Meagher, R. B. (2006b). The shoot-specific expression of gamma-glutamylcysteine synthetase directs the long-distance transport of thiol-peptides to roots conferring tolerance to mercury and arsenic. Plant Physiol 141, 288-98.
Li, Y., Dhankher, O., Carreira, L., Balish, R., and Meagher, R. (2005). Engineered overexpression of gamma-glutamylcysteine synthetase in plants confers high level arsenic and mercury tolerance. Env Tox & Chem 24, 1376-1386.
Li, Y., Dhankher, O., Carreira, L., Lee, D., Chen, A., Schroeder, J., Balish, R., and Meagher, R. (2004). Overexpression of phytochelatin synthase in Arabidopsis leads to enhanced arsenic tolerance and cadmium sensitivity. Plant & Cell Physiol. 45, 1787-1797.
Li, Y., Heaton, A. C. P., Dhankher, O. P., and Meagher, R. B. (2006c). Overexpression of gamma-glutamylcysteine synthetase and glutathione synthetase in plants confers arsenic and mercury resistance. Physiol Plantarum In Press.,
Liu, X. S., and Dean, D. H. (2006). Redesigning Bacillus thuringiensis Cry1Aa toxin into a mosquito toxin. Protein Eng Des Sel 19, 107-11.
Luo, K., Sangadala, S., Masson, L., Mazza, A., Brousseau, R., and Adang, M. J. (1997). The Heliothis virescens 170 kDa aminopeptidase functions as “receptor A” by mediating specific Bacillus thuringiensis Cry1A delta-endotoxin binding and pore formation. Insect Biochem. Mol. Biol 27, 735-43
Luo, K., Banks, D., and Adang, M. J. (1999). Toxicity, binding, and permeability analyses of four Bacillus thuringiensis Cry1A delta-endotoxins using brush border membrane vesicles of Spodoptera exigua and Spodoptera frugiperda. Appl Environ Microbiol 65, 457-64.
Lyyra, S., Meagher, R. B., Kim, T., Heaton, A., Montello, P., Balish, R. S., and Merkle, S. A. (2007). Coupling two mercury resistance genes in Eastern cottonwood enhances the processing of organomercury. Plant Biotechnol. J 5, 254-62.
Majori, G., Ali, A., and Sabatinelli, G. (1987). Laboratory and field efficacy of Bacillus thuringiensis var. Israelensis and Bacillus sphaericus against Anopheles gambiae s.l. and Culex quinquefasciatus in Ouagadougou, Burkina Faso. J Am Mosq Control Assoc 3, 20-5.
Markossian, K. A., Zamyatnin, A. A., and Kurganov, B. I. (2004). Antibacterial proline-rich oligopeptides and their target proteins. Biochemistry (Mosc) 69, 1082-91.
McDowell, J. M., An, Y.-Q., McKinney, E. C., Huang, S., and Meagher, R. B. (1996). The Arabidopsis ACT7 actin gene is expressed in rapidly developing tissues and responds to several external stimuli. Plant Physiol 111, 699-711.
McNall, R. J., and Adang, M. J. (2003). Identification of novel Bacillus thuringiensis Cry1Ac binding proteins in Manduca sexta midgut through proteomic analysis. Insect Biochem. Mol Biol 33, 999-1010.
Meagher, R. B., McKinney, E. C., and Vitale, A. V. (1999). The evolution of new structures: clues from plant cytoskeletal genes. Trends Genet 15, 278-284.
Meagher, R. B., Rugh, C. L., Kandasamy, M. K., Gragson, G., and Wang, N. J. (2000). Engineered phytoremediation of mercury pollution in soil and water using bacterial genes. In: Phytoremediation of Contaminated Soil and Water, N. Terry, and G. Banuelos, eds (Boca Raton: Lewis Publishers), pp. 203-221.
Meagher, R. B., Tait, R. C., Betlach, M., and Boyer, H. W. (1977). Protein expression in E. coli minicells by recombinant plasmids. Cell 10, 521-36.
Merritt, R. W., Dadd, R. H., and Walker, E. D. (1992). Feeding behavior, natural food, and nutritional relationships of larval mosquitoes. Arum Rev Entomol 37, 349-76.
Miller, R. W. (1990). Use of ivermectin to control the lesser mealworm (Coleoptera: Tenebrionidae) in a simulated poultry broiler house. Poult Sci 69, 1281-4.
Morales, C. A., Gast, R., and Guard-Bouldin, J. (2006). Linkage of avian and reproductive tract tropism with sequence divergence adjacent to the 5S ribosomal subunit rrfH of Salmonella enterica. FEMS Microbiol Lett 264, 48-58.
d Morales, C. A., Musgrove, M., Humphrey, T. J., Cates, C., Gast, R., and Guard-Bouldin, J. (2007). Pathotyping of Salmonella enterica by analysis of single-nucleotide polymorphisms in cyaA and flanking 23S ribosomal sequences. Environ Microbiol 9, 1047-59.
Nagpal, S., Gupta, V., Kaur, K. J., and Salunke, D. M. (1999). Structure-function analysis of tritrypticin, an antibacterial peptide of innate immune origin. J Biol Chem 274, 23296-304.
O'Neill, C. M., Gill, S., Hobbs, D., Morgan, C., and Bancroft, I. (2003). Natural variation for seed oil composition in Arabidopsis thaliana. Phytochemistry 64, 1077-90.
Osborn, F. R., Herrera, M. J., Gomez, C. J., and Salazar, A. (2007). Comparison of two commercial formulations of Bacillus thuringiensis var. israelensis for the control of Anopheles aquasalis (Diptera: Culicidae) at three salt concentrations. Mem Inst Oswaldo Cruz 102, 69-72.
Otvos, L., Jr., Wade, J. D., Lin, F., Condie, B. A., Hanrieder, J., and Hoffmann, R. (2005). Designer antibacterial peptides kill fluoroquinolone-resistant clinical isolates. J Med Chem 48, 5349-59.
Otvos, L., Jr. (2002). The short proline-rich antibacterial peptide family. Cell Mol Life Sci 59, 1138-50.
Pawloski, L. C., Deal, R. B., McKinney, E. C., Burgos-Rivera, B., and Meagher, R. B. (2005). Inverted repeat PCR for the rapid assembly of constructs to induce RNA interference. Plant & Cell Physiol 46, 1872-1878.
Qiu, X. Q., Wang, H., Lu, X. F., Zhang, J., Li, S. F., Cheng, G., Wan, L., Yang, L., Zuo, J. Y., Zhou, Y. Q., Wang, H. Y., Cheng, X., Zhang, S. H., Ou, Z. R., Zhong, Z. C., Cheng, J. Q., Li, Y. P., and Wu, G. Y. (2003). An engineered multidomain bactericidal peptide as a model for targeted antibiotics against specific bacteria. Nat Biotechnol. 21, 1480-5.
Rugh, C. L., Wilde, H. D., Stack, N. M., Thompson, D. M., Summers, A. O., and Meagher, R. B. (1996). Mercuric ion reduction and resistance in transgenic Arabidopsis thaliana plants expressing a modified bacterial merA gene. Proc Natl Acad Sci USA 93, 3182-7.
Salin, C., Delettre, Y. R., and Vernon, P. (2003). Controlling the mealworm Alphitobius diaperinus (Coleoptera: Tenebrionidae) in broiler and turkey houses: field trials with a combined insecticide treatment: insect growth regulator and pyrethroid. J Econ Entomol 96, 126-30.
Shakoori, A. R., Anwar, S., Khurshed, N., and Riaz ul, H. (1999). Biocidal activity of Bacillus species for Anopheles larvae. Folia Biol (Krakow) 47, 143-8.
Sundararaj, R., and Rao, D. R. (1993). Field evaluation of a microgel droplet formulation of Bacillus sphaericus 1593M (Biocide-S) against Anopheles culicifacies and Anopheles subpictus in south India. Southeast Asian J Trop Med Public Health 24, 363-8.
Suzuki, A., Terai, H., Toyoda, H., Namikawa, T., Yokota, Y., Tsunoda, T., and Takaoka, K. (2006). A biodegradable delivery system for antibiotics and recombinant human bone morphogenetic protein-2: A potential treatment for infected bone defects. J Orthop Res 24, 327-32.
Tsang, E. W., Yang, J., Chang, Q., Nowak, G., Kolenovsky, A., McGregor, D. I., and Keller, W. A. (2003). Chlorophyll reduction in the seed of Brassica napus with a glutamate 1-semialdehyde aminotransferase antisense gene. Plant Mol Biol 51, 191-201.
Tzen, J. T., Peng, C. C., Cheng, D. J., Chen, E. C., and Chiu, J. M. (1997). A new method for seed oil body purification and examination of oil body integrity following germination. J Biochem. (Tokyo) 121, 762-8.
van Rooijen, G. J., and Moloney, M. M. (1995). Plant seed oil-bodies as carriers for foreign proteins. Biotechnology (N Y) 13, 72-7.
Vitale, A., Wu, R. J., Cheng, Z., and Meagher, R. B. (2003). Multiple conserved 5′ elements are required for high-level pollen expression of the Arabidopsis reproductive actin ACT1. Plant Mol Biol 52, 1135-51.
Wang, H., Guo, J., Lambert, K. N., and Lin, Y. (2007). Developmental control of Arabidopsis seed oil biosynthesis. Planta 226, 773-83.
Weder, J. K., Haussner, K., and Bokor, M. V. (1993). Use of fluorogenic substrates to visualize trypsin and chymotrypsin inhibitors after electrophoresis. Electrophoresis 14, 220-6.
Weiser, J. (1975). Significant recent advances in biological control of vector insects. Adv. Vet Sci Comp Med 19, 47-72.
Wirth, M. C., Yang, Y., Walton, W. E., Federici, B. A., and Berry, C. (2007). Mtx toxins synergize Bacillus sphaericus and Cry11Aa against susceptible and insecticide-resistant Culex quinquefasciatus larvae. Appl Environ Microbiol 73, 6066-71.
Ye, G., Stone, D., Pang, S., Creely, W., Gonzalez, K., and Hinchee, M. (1999). Arabidopsis ovule is the target for Agrobacterium in planta vacuum infiltration transformation. The Plant Journal 19, 249-257.
Zhang, W., Qi, W., Albert, T. J., Motiwala, A. S., Alland, D., Hyytia-Trees, E. K., Ribot, E. M., Fields, P. I., Whittam, T. S., and Swaminathan, B. (2006). Probing genomic diversity and evolution of Escherichia coli 0157 by single nucleotide polymorphisms. Genome Res 16, 757-67.
Zimmerman, M., Ashe, B., Yurewicz, E. C., and Patel, G. (1977). Sensitive assays for trypsin, elastase, and chymotrypsin using new fluorogenic substrates. Anal Biochem. 78, 47-51.

1. A plant flour material comprising a ground, dried plant material, wherein the plant material contains a recombinant protein comprising a bactericidal or an insecticidal protein toxin, or combinations thereof.

2. The plant flour material of claim L wherein the bactericidal toxin is Bt or Bti.

3. (canceled)

4. The plant flour material of claim 1, further comprising BtB.

5. (canceled)

6. The plant flour material of claim 1, wherein the insecticidal toxin is a proline-rich antibacterial peptide (PRAP) protein.

7. The plant flour material of claim 1, wherein the composition comprises a mixture of at least three flours, wherein a first flour contains a Bt toxin, a second flour contains a BtB, and a third flour contains an insecticidal protein.

8. (canceled)

9. The plant flour material of claim 1, wherein the ground, dried plant material is a seed.

10. The plant flour material of claim 9, wherein the flour material comprises seed oil bodies.

11. The plant flour material of claim 10, wherein the seed oil bodies comprise an oil body protein fused to the bactericidal or insecticidal protein toxin.

12. (canceled)

13. A seed oil body composition comprising the oil body protein fusion of claim 18.

14. The seed oil body composition of claim 13, wherein the bactericidal toxin is Bt or Bti.

15. The seed oil body composition of claim 13, further comprising BtB.

16. (canceled)

17. The seed oil body composition of claim 13, wherein the seed oil in the seed oil body is canola oil.

18. An oil body protein (OBP) fusion, comprising an oil body protein fused to a recombinant protein comprising a bactericidal or an insecticidal protein toxin.

19. The oil body protein fusion of claim 18, wherein operably positioned between the oil body protein and the recombinant protein is a protease or chymotrypsin cassette.

20. The oil body protein fusion of claim 19, wherein the protease or chymotrypsin cassette comprises a conserved protease or chymotrypsin sequence.

21. (canceled)

22. The seed oil body composition of claim 13, wherein the oil bodies in the composition have a buoyant density of about 0.8 g per mL to about 1.1 g per mL.

23. A method of abating or controlling a pest insect population comprising administering to the pest insect population a food source or a water source comprising a recombinant plant material containing two or more recombinant proteins comprising a bactericidal or an insecticidal protein toxin, or combinations thereof.

24. (canceled)

25. (canceled)

26. The method of claim 23, wherein the recombinant plant material comprises recombinant material from a first recombinant plant expressing a bactericidal protein toxin and a second recombinant plant expressing an the insecticidal protein toxin.

27. The method of claim 23, wherein the bactericidal protein toxin is a proline-rich antibacterial peptide (PRAP) protein.

28. The method of claim 23, wherein the recombinant plant material comprises a seed oil body preparation comprising one or more Bti toxins, optionally in combination with one or more BtBs.


SEQUENCE LISTING (12 sequences)

SEQ ID NO: 1 - DNA, 3737 bp, artificial sequence
  synthetic B.t.-toxin expression construct A2pt::Cry1Ac
ggtacctgatctcaaatacattgatacatatctcatctagatctaggttatcattatgtaagaaagttttgacgaatatggcacgacaaaatggctagactcgatgtaattggtatctcaactcaacattatacttataccaaacattagttagacaaaatttaaacaactattttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaattgtctcgttgtcctcctcactttcatcagccgttttgaatctccggcgacttgacagagaagaacaaggaagaagactaagagagaaagtaagagataatccaggagattcattctccgttttgaatcttcctcaatctcatcttcttccgctctttctttccaaggtaataggaactttctggatctactttatttgctggatctcgatcttgttttctcaatttccttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaaggttagatgaagtttgtgtatagattcttcgaaactttaggatttgtagtgtcgtacgttgaacagaaagctatttctgattcaatcagggtttatttgactgtattgaactctttttgtgtgtttgcagctcataaaccatggacaacaatcccaacatcaacgagtgcattccttacaactgcctgagcaaccctgaggttgaggtgctgggtggagaacggattgagactggttacacacctatcgacatctcgttgtcacttacccaattccttttgtcagagttcgtgcccggtgctggattcgtgcttggacttgtcgatatcatttggggaatctttggtccctctcaatgggacgcctttcttgtacagatagagcaacttatcaaccaaaggattgaagagttcgctaggaaccaagccatctcaaggttagaaggcctcagcaacctttaccagatttacgcagaatcttttcgagagtgggaagcagacccgaccaatcctgccttaagagaggagatgcgcattcaattcaatgacatgaacagcgcgctgacgaccgcaattccgctcttcgccgttcagaattaccaagttcctcttttatccgtgtacgtgcaggctgccaacctgcacttgtcggtgctccgcgatgtctccgtgttcggacaacggtggggctttgatgccgcaactatcaatagtcgttataatgatctgactaggcttattggcaactataccgattatgctgttcgctggtacaacacgggtctcgaacgtgtctggggaccggattctagagattgggtcaggtacaaccagttcaggcgagagttgacactaactgtcctagacattgtcgctctctttcccaactacgactctaggcgctacccaatccgtactgtgtcacaattgacccgggaaatctacacaaacccagtcctcgagaacttcgacggtagctttcgaggctcggctcagggcatagagagaagcatcaggtctccacacctgatggacatattgaacagtatcacgatctacaccgatgcgcaccgcggttattactactggtcagggcatcagatcatggcatcacccgttgggttctctggaccagaattcactttcccactttacgggactatgggcaatgcagctccacaacaacgtattgttgctcaactcggtcagggcgtgtatagaaccttgtccagcactctatataggagacctttcaacatcggcatcaacaatcaacaattgtctgtgcttgacgggacagaatttgcctatggaacctcctcaaatctgccatccgctgtctacagaaagagcggaacagttgatagcttggatgagatccctccacagaacaacaacgttccacctaggcaagggtttagccatcgccttagccatgtgtccatgttccgttcaggctttagtaatagcagcgttagtatcatcagagctccgatgttctcttggatacatcgtagtgctgagtttaacaacataattgcatccgatagcattactcagatcccagctgtcaaggggaactttctctttaatggttctgtcatttcaggaccaggattcactggaggcgacttggggaactttctctttaatggttctgtcatttcaggaccaggattcactggaggcgacttggacttcccatcgacatctaccagatatcgtgttcgtgtaaggtatgcctctgttacccctattcacctcaacgtcaattggggtaattcctccatcttttccaatacagtaccagcgacagctacatccttggataatctccaatctagcgatttcggttacttcgaaagtgccaatgccttcacctcttccctaggtaacatagtaggtgttagaaatttctccggaaccgccggagtgataatcgaccgcttcgaattcattcccgttactgcaacgctcgagtaatagggatcctctagactcggaggctctcaagatcaaaggcttaaaaagctggggttttatgaatgggatcaaagtttctttttttcttttatatttgcttctccatttgtttgtttcatttccctttttgttttcgtttctatgatgcacttgtgtgtgacaaactctctgggtttttacttacgtctgcgtttcaaaaaaaaaaaccgctttcgttttgcgttttagtcccattgttttgtagctctgagtgatcgaattgatgcctctttattccttttgttccctataatttctttcaaaactcagaagaaaaaccttgaaactctttgcaatgttaatataagtattgtataagatttttattgatttggttattagtcttacttttgctacctccatcttcacttggaactgatattctgaatagttaaagcgttacatgtcttccattcacaaatgaacttaaactagcacaaagtcagatattttaagaccgcggtggagctc

SEQ ID NO: 2 - protein, 613 aa, artificial sequence
  Synthetic Construct
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLGNFLFNGSVISGPGFTGGDLDFPSTSTRYRVRVRYASVTPIHLNVNWGNSSIFSNTVPATATSLDNLQSSDFGYFESANAFTSSLGNIVGVRNFSGTAGVIIDRFEFIPVTATLE

SEQ ID NO: 3 - DNA, 3383 bp, artificial sequence
  synthetic B.t.Booster expression construct A2pt::CR9-MPED
ggtacctgatctcaaatacattgatacatatctcatctagatctaggttatcattatgtaagaaagttttgacgaatatggcacgacaaaatggctagactcgatgtaattggtatctcaactcaacattatacttataccaaacattagttagacaaaatttaaacaactattttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaattgtctcgttgtcctcctcactttcatcagccgttttgaatctccggcgacttgacagagaagaacaaggaagaagactaagagagaaagtaagagataatccaggagattcattctccgttttgaatcttcctcaatctcatcttcttccgctctttctttccaaggtaataggaactttctggatctactttatttgctggatctcgatcttgttttctcaatttccttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaaggttagatgaagtttgtgtatagattcttcgaaactttaggatttgtagtgtcgtacgttgaacagaaagctatttctgattcaatcagggtttatttgactgtattgaactctttttgtgtgtttgcagctcataaaccatggcagacctcaagggatattgggggacgtacgctatacatatacgggcattcgaccacggcattccgcaaatgtccatgaacgagacatatgagctgattatccatccgttcaactactacgcgcctgagttcgtcttcccgaccaacgatgccgtcatacgacttgcgagggaacgagctgtaatcaatggagttctagcgacagtgaacggagagttcttggagcggatatcggcgactgacccggacggactccacgcgggcgtcgtcaccttccaagtggtaggcgatgaggaatcacaacggtactttcaagtagttaacgatggcgcgaacctcggctcgttgaggttactgcaagccgttccagaggagatcagggagttccggataacgattcgcgctacagaccagggaacggacccaggaccgctgtccacggacatgacgttcagagttgtttttgtgcccacgcaaggagaacctagattcgcgtcctcagaacatgctgtcgctttcatagaaaagagtgccggcatggaagagtctcaccaacttcctctagcacaagacatcaagaaccatctctgtgaagacgactgtcacagcatttactatcgtattatcgatggcaacagcgagggtcatttcggcctggatcctgttcgcaacaggttgttcctgaagaaagagctgataagagaacaaagtgcctcccacactctgcaagtggcggctagtaactcgcccgatggtggcattccacttcctgcttccatccttactgtcactgttaccgtgagggaggcagaccctcgtccagtgtttatgagggaattgtacaccgcagggatatccacagcggactccatcggcagagagctgctcagattacatgcgacccagtctgaaggcgcggccattacttatgctatagactacgatacaatggtagtggaccccagcctggaggcagtgagacagtcggctttcgtactgaacgctcaaaccggagtgctgacgcttaatatccagcccacggccacgatgcatggactgttcaaattcgaagtcacagctactgacacggccggcgctcaggaccgcaccgacgtcaccgtgtacgtggtatcctcgcagaaccgcgtctacttcgtgttcgtcaacacgctgcaacaggtcgaagacaacagagactttatcgcggacaccttcagcgctgggttcaacatgacctgcaacatcgaccaagtggtgcccgccaacgaccccgtcaccggcgtggcgctggagcacagcacgcagatgcgcggccacttcatacgggacaacgtacccgtactcgctgatgagatagaacagatccgtagtgacctagtcctcctgagctcgatacaaacaacgctggcggcgcgatcgctggtgttgcaggacttgttgaccaactccagcccggactcggcgcctcaccaccatcaccaccattaaagatcctctagactcggaggctctcaagatcaaaggcttaaaaagctggggttttatgaatgggatcaaagtttctttttttcttttatatttgcttctccatttgtttgtttcatttccctttttgttttcgtttctatgatgcacttgtgtgtgacaaactctctgggtttttacttacgtctgcgtttcaaaaaaaaaaaccgctttcgttttgcgttttagtcccattgttttgtagctctgagtgatcgaattgatgcctctttattccttttgttccctataatttctttcaaaactcagaagaaaaaccttgaaactctttgcaatgttaatataagtattgtataagatttttattgatttggttattagtcttacttttgctacctccatcttcacttggaactgatattctgaatagttaaagcgttacatgtcttccattcacaaatgaacttaaactagcacaaagtcagatattttaagaccgcggtggagctc

SEQ ID NO: 4 - protein, 496 aa, artificial sequence
  Synthetic Construct
MADLKGYWGTYAIHIRAFDHGIPQMSMNETYELIIHPFNYYAPEFVFPTNDAVIRLARERAVINGVLATVNGEFLERISATDPDGLHAGVVTFQVVGDEESQRYFQVVNDGANLGSLRLLQAVPEEIREFRITIRATDQGTDPGPLSTDMTFRVVFVPTQGEPRFASSEHAVAFIEKSAGMEESHQLPLAQDIKNHLCEDDCHSIYYRIIDGNSEGHFGLDPVRNRLFLKKELIREQSASHTLQVAASNSPDGGIPLPASILTVTVTVREADPRPVFMRELYTAGISTADSIGRELLRLHATQSEGAAITYAIDYDTMVVDPSLEAVRQSAFVLNAQTGVLTLNIQPTATMHGLFKFEVTATDTAGAQDRTDVTVYVVSSQNRVYFVFVNTLQQVEDNRDFIADTFSAGFNMTCNIDQVVPANDPVTGVALEHSTQMRGHFIRDNVPVLADEIEQIRSDLVLLSSIQTTLAARSLVLQDLLTNSSPDSAPHHHHHH

SEQ ID NO: 5 - DNA, 2921 bp, artificial sequence
  synthetic Oleosin GFP fusion expression construct A7pt::OL1-GFP
ggtacctctaaattgctaataaacgaccatttccgtcaattctccttggttgcaacagtctacccgtcaaatgtttactaatttataagtgtgaagtttgaattatgaaaaacgaaatcgtattaaaaattcacaagaataaacaactccatagattttcaaaaaaacagtcacgagaaaaaaaccacagaccgtttgtctgctcttctagtttttattatttttctattaatagttttttgttatttcgagaataaaatttgaacgatgtccgaaccacaaaagccgagccgataaatcctaagccgagcctaactttagccgtaaccatcagtcacggctcccgggctaattcatttgaaccgaatcataatcaacggtttagatcaaactcaaaacaatctaacggcaacatagacgcgtcggtgagctaaaaagagtgtgaaagccaggtcaccatagcattgtctctcccagattttttatttgggaaataatagaagaaatagaaaaaaataaaagagtgagaaaaatcgtagagctatatattcgcacatgtactcgtttcgctttccttagtgttagctgctgccgctgttgtttctcctccatttctctatctttctctctcgctgcttctcgaatcttctgtatcatcttcttcttcttcaaggtgagtctctagatccgttcgcttgattttgctgctcgttagtcgttattgttgattctctatgccgatttcgctagatctgtttagcatgcgttgtggttttatgagaaaatctttgttttgggggttgcttgttatgtgattcgatccgtgcttgttggatcgatctgagctaattcttaaggtttatgtgttagatctatggagtttgaggattcttctcgcttctgtcgatctctcgctgttatttttgtttttttcagtgaagtgaagttgtttagttcgaaatgacttcgtgtatgctcgattgatctggttttaatcttcgatctgttaggtgttgatgtttacaagtgaattctagtgttttctctttgagatctgtgaagtttgaacctagttttctcaataatcaacatatgaagcgatgtttgagtttcaataaacgctgctaatcttcgaaactaagttgtgatctgattcgtgtttacttcatgagcttatccaattcatttcggtttcattttactttttttttagtgaaccatgagtgcggatacagctagaggaacccatcacgacatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtacgcaacgggagagcacccacagggatcagacaagttggacagtgcaaggatgaagttgggaagcaaagctcaggatctgaaagacagagcacagtactacggacagcaacatactggtggggaacatgaccgtgaccgtactcgtggtggccagcacactactggaagtggttcaggtgctgctgcttttggaggaggtggtccagctagattagctggtggagccatggtagatctgactagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccgtggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataaggatccgtgtgtcttgtcttatctggttcgtggtggtgagtttgttacaaaaaaatctattttccctagttgagatgggaattgaactatctgttgttatgtggattttattttcttttttctctttagaaccttatggttgtgtcaagaagtcttgtgtactttagttttatatctctgttttatctcttctattttctttaggatgcttgtgatgatgctgtttttttttgtccctaagcaaaaaaatatcatattatatttggtccttggttcatttttttggtttttttttgtcttcacatataaatattgtttgaatgtcttcaatcttttatttgtatgagacaattatttaagtatcgggtgacaatgcagctatgagctc

SEQ ID NO: 6 - protein, 437 aa, artificial sequence
  Synthetic Construct
MSADTARGTHHDIIGRDQYPMMGRDRDQYQMSGRGSDYSKSRQIAKAATAVTAGGSLLVLSSLTLVGTVIALTVATPLLVIFSPILVPALITVALLITGFLSSGGFGIAAITVFSWIYKYATGEHPQGSDKLDSARMKLGSKAQDLKDRAQYYGQQHTGGEHDRDRTRGGQHTTGSGSGAAAFGGGGPARLAGGAMVDLTSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKTRHNIEDGGVQLADHYQQNTPIGDGPVLLPDNHYLSTQSALSKDPNEKRDHMVLLEFVTAAGITHGMDELYK

SEQ ID NO: 7 - DNA, 4265 bp, artificial sequence
  synthetic Bti-toxin expression construct A7ptOL1-Cry4Ba-GAV
ggtacctctaaattgctaataaacgaccatttccgtcaattctccttggttgcaacagtctacccgtcaaatgtttactaatttataagtgtgaagtttgaattatgaaaaacgaaatcgtattaaaaattcacaagaataaacaactccatagattttcaaaaaaacagtcacgagaaaaaaaccacagaccgtttgtctgctcttctagtttttattatttttctattaatagttttttgttatttcgagaataaaatttgaacgatgtccgaaccacaaaagccgagccgataaatcctaagccgagcctaactttagccgtaaccatcagtcacggctcccgggctaattcatttgaaccgaatcataatcaacggtttagatcaaactcaaaacaatctaacggcaacatagacgcgtcggtgagctaaaaagagtgtgaaagccaggtcaccatagcattgtctctcccagattttttatttgggaaataatagaagaaatagaaaaaaataaaagagtgagaaaaatcgtagagctatatattcgcacatgtactcgtttcgctttccttagtgttagctgctgccgctgttgtttctcctccatttctctatctttctctctcgctgcttctcgaatcttctgtatcatcttcttcttcttcaaggtgagtctctagatccgttcgcttgattttgctgctcgttagtcgttattgttgattctctatgccgatttcgctagatctgtttagcatgcgttgtggttttatgagaaaatctttgttttgggggttgcttgttatgtgattcgatccgtgcttgttggatcgatctgagctaattcttaaggtttatgtgttagatctatggagtttgaggattcttctcgcttctgtcgatctctcgctgttatttttgtttttttcagtgaagtgaagttgtttagttcgaaatgacttcgtgtatgctcgattgatctggttttaatcttcgatctgttaggtgttgatgtttacaagtgaattctagtgttttctctttgagatctgtgaagtttgaacctagttttctcaataatcaacatatgaagcgatgtttgagtttcaataaacgctgctaatcttcgaaactaagttgtgatctgattcgtgtttacttcatgagcttatccaattcatttcggtttcattttactttttttttagtgaaccatgagtgcggatacagctagaggaacccatcacgacatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtacgcaacgggagagcacccacagggatcagacaagttggacagtgcaaggatgaagttgggaagcaaagctcaggatctgaaagacagagcacagtactacggacagcaacatactggtggggaacatgaccgtgaccgtactcgtggtggccagcacactactggaagtggttcaggtgctgctgcttttggaggaggtggtccagctagattagctggtggagccatggctcaacaatatggagttaatcctgcagctatcaatagcagctctgtctctacagcacttaaagtggcaggagctattttaaagtttgtgaatccacctgcaggcacagtgttgactgtactaagtgctgtactgcctatactttggccaactaacacacccactcctgagagagtgtggaatgattttatgactaatactggtaacctcattgaccaaacagtgacagcctatgttagaactgatgctaatgccaagatgactgtggttaaagattatcttgatcaatataccaccaagtttaatacatggaaaagagaaccaaataatcaatcttatagaactgcagttattactcaatttaacctcacctcagctaaacttagagagacagcagtctattttagcaaccttgtgggatatgaactcttgctgttacctatttatgctcaagtagctaatttcaatctcttactaattagagatggtttaattaatgctcaggagtggtctctggcagcttctgcaggtgatcagctctacaacactatggttcaatatactaaggagtacatagcccactctataacttggtataataaaggccttgatgtgttgagaaacaagtctaatggccagtggataacatttaatgactacaagagagaaatgacaatacaggtgttggatatcttagctctctttgctagttatgacccaagaagatacccagcagacaagattgataatactaagctgagcaaaactgagtttacaagagaaatttatactgcccttgttgaaagcccttcttcaaagagtattgctgctttggaagctgcactcactagggatgtgcatctcttcacatggttaaagagagttgatttctggactaatacaatctaccaagatttgaggttcttgtctgcaaataagattggattttcttatactaatagtagtgccatgcaagagagtggtatctatgggtctagtggatttgggtccaatcttacccaccagattcagttgaacagtaatgtgtacaaaacctctattacagacacatcttccccttcaaatagagtgactaaaatggacttctataaaattgatggaacacttgcctcttacaattcaaatatcacacctactcctgaagggcttagaaccacattctttggtttcagtaccaatgagaatacacctaatcaaccaacagtgaatgattacacccatatcctctcatatatcaagacagatgttattggggctgtttacaatagtaatagagtgtcatttgcatggactcataagattgttgatcccaacaatcaaatatacactgatgctattacccaggttccagctgtgaagtcaaacttcttgaatgccacagcaaaggttataaagggtcctggtcatacaggaggggatcttgttgcattgacctctaatgggactctaagtggaagaatggagatccagtgtaaaacatctatcttcaatgatcctacaaggtcttatgggctcaggattagatatgcagctaattcacccattgttctgaatgtgagctatgtgctgcagggagtttctagagggaccactatcagtacagagtcaacattttcaaggccaaacaatataatccctactgacttaaagtatgaagagttcagatataaggacccttttgatgctatagttcctatgagactttcctctaatcaacttataacaattgcaatccaaccactcaacatgaccagcaataatcaggttattatagatagaattgaaattattcctattacccaaagtgtccttgatgaaactgaaaatcaaaacttggagtcagaaagggaagttgtgaatgctcttttcacaaatgatgcaaaggatgctctaaacattggtactacagattatgatatagaccaagcagctaatttagttgaatgtatcagtgaggaattatatccaaaagaaaagatgctcttgttggatgaagtgaagaatgcaaaacagctttcacaatctaggaatgtcttacagaatggttaaggatccgtgtgtcttgtcttatctggttcgtggtggtgagtttgttacaaaaaaatctattttccctagttgagatgggaattgaactatctgttgttatgtggattttattttcttttttctctttagaaccttatggttgtgtcaagaagtcttgtgtactttagttttatatctctgttttatctcttctattttctttaggatgcttgtgatgatgctgtttttttttgtccctaagcaaaaaaatatcatattatatttggtccttggttcatttttttggtttttttttgtcttcacatataaatattgtttgaatgtcttcaatcttttatttgtatgagacaattatttaagtatcgggtgacaatgcagctatgagctc

SEQ ID NO: 8 - protein, 885 aa, artificial sequence
  Synthetic Construct
MSADTARGTHHDIIGRDQYPMMGRDRDQYQMSGRGSDYSKSRQIAKAATAVTAGGSLLVLSSLTLVGTVIALTVATPLLVIFSPILVPALITVALLITGFLSSGGFGIAAITVFSWIYKYATGEHPQGSDKLDSARMKLGSKAQDLKDRAQYYGQQHTGGEHDRDRTRGGQHTTGSGSGAAAFGGGGPARLAGGAMAQQYGVNPAAINSSSVSTALKVAGAILKFVNPPAGTVLTVLSAVLPILWPTNTPTPERVWNDFMTNTGNLIDQTVTAYVRTDANAKMTVVKDYLDQYTTKFNTWKREPNNQSYRTAVITQFNLTSAKLRETAVYFSNLVGYELLLLPIYAQVANFNLLLIRDGLINAQEWSLAASAGDQLYNTMVQYTKEYIAHSITWYNKGLDVLRNKSNGQWITFNDYKREMTIQVLDILALFASYDPRRYPADKIDNTKLSKTEFTREIYTALVESPSSKSIAALEAALTRDVHLFTWLKRVDFWTNTIYQDLRFLSANKIGFSYTNSSAMQESGIYGSSGFGSNLTHQIQLNSNVYKTSITDTSSPSNRVTKMDFYKIDGTLASYNSNITPTPEGLRTTFFGFSTNENTPNQPTVNDYTHILSYIKTDVIGAVYNSNRVSFAWTHKIVDPNNQIYTDAITQVPAVKSNFLNATAKVIKGPGHTGGDLVALTSNGTLSGRMEIQCKTSIFNDPTRSYGLRIRYAANSPIVLNVSYVLQGVSRGTTISTESTFSRPNNIIPTDLKYEEFRYKDPFDAIVPMRLSSNQLITIAIQPLNMTSNNQVIIDRIEIIPITQSVLDETENQNLESEREVVNALFTNDAKDALNIGTTDYDIDQAANLVECISEELYPKEKMLLLDEVKNAKQLSQSRNVLQNG

SEQ ID NO: 9 - DNA, 3203 bp, artificial sequence
  synthetic BtBooster expression construct A7pt::OL1-AgCad
ggtacctctaaattgctaataaacgaccatttccgtcaattctccttggttgcaacagtctacccgtcaaatgtttactaatttataagtgtgaagtttgaattatgaaaaacgaaatcgtattaaaaattcacaagaataaacaactccatagattttcaaaaaaacagtcacgagaaaaaaaccacagaccgtttgtctgctcttctagtttttattatttttctattaatagttttttgttatttcgagaataaaatttgaacgatgtccgaaccacaaaagccgagccgataaatcctaagccgagcctaactttagccgtaaccatcagtcacggctcccgggctaattcatttgaaccgaatcataatcaacggtttagatcaaactcaaaacaatctaacggcaacatagacgcgtcggtgagctaaaaagagtgtgaaagccaggtcaccatagcattgtctctcccagattttttatttgggaaataatagaagaaatagaaaaaaataaaagagtgagaaaaatcgtagagctatatattcgcacatgtactcgtttcgctttccttagtgttagctgctgccgctgttgtttctcctccatttctctatctttctctctcgctgcttctcgaatcttctgtatcatcttcttcttcttcaaggtgagtctctagatccgttcgcttgattttgctgctcgttagtcgttattgttgattctctatgccgatttcgctagatctgtttagcatgcgttgtggttttatgagaaaatctttgttttgggggttgcttgttatgtgattcgatccgtgcttgttggatcgatctgagctaattcttaaggtttatgtgttagatctatggagtttgaggattcttctcgcttctgtcgatctctcgctgttatttttgtttttttcagtgaagtgaagttgtttagttcgaaatgacttcgtgtatgctcgattgatctggttttaatcttcgatctgttaggtgttgatgtttacaagtgaattctagtgttttctctttgagatctgtgaagtttgaacctagttttctcaataatcaacatatgaagcgatgtttgagtttcaataaacgctgctaatcttcgaaactaagttgtgatctgattcgtgtttacttcatgagcttatccaattcatttcggtttcattttactttttttttagtgaaccatgagtgcggatacagctagaggaacccatcacgacatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtacgcaacgggagagcacccacagggatcagacaagttggacagtgcaaggatgaagttgggaagcaaagctcaggatctgaaagacagagcacagtactacggacagcaacatactggtggggaacatgaccgtgaccgtactcgtggtggccagcacactactggaagtggttcaggtgctgctgcttttggaggaggtggtccagctagattagctggtggagccatggctggcagccctctcccaaagtttgaagcaattgatgatgatggtggaatatatggggatgtgacattctctcttacatctacaaatgatggtgagcaagatcatgaagtctttagagttgacaaggtggacaataaaactggcctcttagtactggagaactctctagctgtgcagccttttcctaagaattactctatcactgtaattgccagagatggaggagatagacaatctgaggcagctatacatgttgtctttattaacatgacaggtgaaccagctttccttgagccaacatttgatacagattttactgagaatgaagaaggcagggatgagaggagacagctcccatttgctgaagatcctaagaatgcaggattacctccaggagctgaaactaatgtatactattttattgataaaacttatggaaatgcttctcacttgttccaacttgatagagttagtaacgttcttcaactagctcagttacttgatagagaggaaatacctacaccagaaataagaattgttgctacaaacaatgagaatagccctcctgataccgtgttggagtctagcccaagcctcttagttgtgaggattaaggtgaatgatgttaatgacaatcctcctgttttccagcaaagactgtatgcagcaggaattacaactaatgacagagttccaaaagcactcttcagggtttacgctgaagaccctgatgaggatgaaatcataaggtatgagcttgtaaatgggactgcagtcggggaaaaccttcaaactgatggacttcctttcaggcttcatccagattctggtgaactgactctaacatctaaggttcaaccaaaccagaatggatattaccaattgactctaatagcctttgatagggatgatactcacaatgacactgtacctgctaaggtttatattgttagtgaatcaaatagagttacatttgtctttcttgagcaccaccaccatcatcattaaggatccgtgtgtcttgtcttatctggttcgtggtggtgagtttgttacaaaaaaatctattttccctagttgagatgggaattgaactatctgttgttatgtggattttattttcttttttctctttagaaccttatggttgtgtcaagaagtcttgtgtactttagttttatatctctgttttatctcttctattttctttaggatgcttgtgatgatgctgtttttttttgtccctaagcaaaaaaatatcatattatatttggtccttggttcatttttttggtttttttttgtcttcacatataaatattgtttgaatgtcttcaatcttttatttgtatgagacaattatttaagtatcgggtgacaatgcagctatgagctc

SEQ ID NO: 10 - protein, 531 aa, artificial sequence
  Synthetic Construct
MSADTARGTHHDIIGRDQYPMMGRDRDQYQMSGRGSDYSKSRQIAKAATAVTAGGSLLVLSSLTLVGTVIALTVATPLLVIFSPILVPALITVALLITGFLSSGGFGIAAITVFSWIYKYATGEHPQGSDKLDSARMKLGSKAQDLKDRAQYYGQQHTGGEHDRDRTRGGQHTTGSGSGAAAFGGGGPARLAGGAMAGSPLPKFEAIDDDGGIYGDVTFSLTSTNDGEQDHEVFRVDKVDNKTGLLVLENSLAVQPFPKNYSITVIARDGGDRQSEAAIHVVFINMTGEPAFLEPTFDTDFTENEEGRDERRQLPFAEDPKNAGLPPGAETNVYYFIDKTYGNASHLFQLDRVSNVLQLAQLLDREEIPTPEIRIVATNNENSPPDTVLESSPSLLVVRIKVNDVNDNPPVFQQRLYAAGITTNDRVPKALFRVYAEDPDEDEIIRYELVNGTAVGENLQTDGLPFRLHPDSGELTLTSKVQPNQNGYYQLTLIAFDRDDTHNDTVPAKVYIVSESNRVTFVFLEHHHHHH

SEQ ID NO: 11 - DNA, 1661 bp, artificial sequence
  synthetic Formaecin Is expression construct A7pt::FormIs
ggtacctctaaattgctaataaacgaccatttccgtcaattctccttggttgcaacagtctacccgtcaaatgtttactaatttataagtgtgaagtttgaattatgaaaaacgaaatcgtattaaaaattcacaagaataaacaactccatagattttcaaaaaaacagtcacgagaaaaaaaccacagaccgtttgtctgctcttctagtttttattatttttctattaatagttttttgttatttcgagaataaaatttgaacgatgtccgaaccacaaaagccgagccgataaatcctaagccgagcctaactttagccgtaaccatcagtcacggctcccgggctaattcatttgaaccgaatcataatcaacggtttagatcaaactcaaaacaatctaacggcaacatagacgcgtcggtgagctaaaaagagtgtgaaagccaggtcaccatagcattgtctctcccagattttttatttgggaaataatagaagaaatagaaaaaaataaaagagtgagaaaaatcgtagagctatatattcgcacatgtactcgtttcgctttccttagtgttagctgctgccgctgttgtttctcctccatttctctatctttctctctcgctgcttctcgaatcttctgtatcatcttcttcttcttcaaggtgagtctctagatccgttcgcttgattttgctgctcgttagtcgttattgttgattctctatgccgatttcgctagatctgtttagcatgcgttgtggttttatgagaaaatctttgttttgggggttgcttgttatgtgattcgatccgtgcttgttggatcgatctgagctaattcttaaggtttatgtgttagatctatggagtttgaggattcttctcgcttctgtcgatctctcgctgttatttttgtttttttcagtgaagtgaagttgtttagttcgaaatgacttcgtgtatgctcgattgatctggttttaatcttcgatctgttaggtgttgatgtttacaagtgaattctagtgttttctctttgagatctgtgaagtttgaacctagttttctcaataatcaacatatgaagcgatgtttgagtttcaataaacgctgctaatcttcgaaactaagttgtgatctgattcgtgtttacttcatgagcttatccaattcatttcggtttcattttactttttttttagtgaaccatgggtcgtccaaaccctgttcctaacccaaaacctccacatccacgtctgtaaggatccgtgtgtcttgtcttatctggttcgtggtggtgagtttgttacaaaaaaatctattttccctagttgagatgggaattgaactatctgttgttatgtggattttattttcttttttctctttagaaccttatggttgtgtcaagaagtcttgtgtactttagttttatatctctgttttatctcttctattttctttaggatgcttgtgatgatgctgtttttttttgtccctaagcaaaaaaatatcatattatatttggtccttggttcatttttttggtttttttttgtcttcacatataaatattgtttgaatgtcttcaatcttttatttgtatgagacaattatttaagtatcgggtgacaatgcagctatgagctc

SEQ ID NO: 12 - protein, 17 aa, artificial sequence
  Synthetic Construct
MGRPNPVPNPKPPHPRL